(19) United States
(12) Patent Application Publication
(10) Pub. No.: US 2003/0130484 A1
(43) Pub. Date: Jul. 10, 2003

(54) Inhibitors and disassemblers of fibrillogenesis

(76) Inventors: David J. Gordon, Chicago, IL (US); Stephen C. Meredith, Chicago, IL (US)

Correspondence Address: BARNES & THORNBURG, 2600 CHASE PLAZA, 10 LASALLE STREET, CHICAGO, IL, 60603

(21) Appl. No.: 10/103,658

(22) Filed: Mar. 20, 2002

Related U.S. Application Data
(60) Provisional application No. 60/277,477, filed on Mar. 20, 2001.

(51) Int. Cl. A61K 38/17; C07K 14/435; G01N 33/53

(52) U.S. Cl. 530/350; 514/12; 435/7.1

(57) ABSTRACT

Methods and compositions are presented that inhibit fibril formation and/or bring about disassembly of pre-formed fibrils. Compositions include peptides with short β-strands with two faces: one that can bind to β-amyloids through hydrogen bonds, and one which blocks propagation of hydrogen bonding needed to form fibrils. Thus, short congeners of the fibril protein containing N-methyl amino acids or esters are provided for the inhibition of fibril formation and for the disassembly of pre-existing or pre-formed fibrils. Specific aspects address β-amyloid fibrils; prion mediated fibrils; Huntington protein fibrils. Methods for screening for potential fibril inhibitors and disassemblers, diagnostic analysis and treatments are provided.

This invention claims priority from U.S. Serial No. 60/277,477 filed Mar. 20, 2001 incorporated herein by reference.

The government owns rights in the present invention pursuant to grant number T32 GM07281 from the National Institutes of Health. This work was also supported by the Alzheimer's Association Grant IIRG# 98-1344.

### BACKGROUND OF THE INVENTION

Methods and compositions are presented that are usefull for treatment of pathologies associated with fibrillogenesis. Peptide inhibitors block fibril formation and/or dissemble pre-formed fibrils. Screening tests for inhibitors, and their diagnostic and therapeutic uses, are presented.

Fibrillogenesis is the cause of various pathologies, especially those involving neuronal degeneration. Different fibril forming proteins are involved in these pathologies, and fibril formation is followed by deposition of these insoluble fibrils in tissues. Generally, fibrillogenesis leads to formation of plaques and tangles, and eventual cellular degeneration as the pathology progresses. Despite a lack of amino acid sequence homology, these different fibril forming proteins are all believed to have β-sheet conformations (Carrel and Lomas, 1997; Horwich et al., 1997).

Amyloidosis is defined as the deposition of amyloid fibrils into tissues, and is typified in diseases such as Alzheimer's Disease (AD) and Down's Syndrome. Systemic amyloidosis is characterized by amyloid deposition throughout the viscera. Animal amyloid is a complex material composed mainly of protein fibrils. The protein that comprises these fibrils varies from disease to disease. β-amyloid proteins are involved in the pathological progression of Alzheimer's Disease (AD) (Glenner and Wong, 1984).

Alzheimer's Disease, Huntington's Disease, systemic amyloidoses and prion diseases, among others, all share the common characteristic of aggregation of peptides and proteins into insoluble amyloid fibrils (Koo, 1998; Kelly, 2000). The aggregating proteins in these diseases include the AD peptide in Alzheimer's Disease, huntingtin in Huntington's Disease, the scrapie form of the prion protein (PrP) in the transmissible spongiform encephalopathies and transthyretin in some forms of familial amyloidoses. Despite a lack of structural similarity between these soluble proteins, the amyloid fibrils share many common characteristics, including protease resistance and extensive β-sheet structure (Sipe, 1992; Inouye, 1993). In addition, amyloid fibrils formed from different proteins exhibit similar fiber diffraction patterns and also interact with the dyes Congo Red and thioflavin T (Sipe, 1992; Naiki, 1989; Klunk, 1989). In spite of these similarities, recent solid state NMR experiments with intact Aβ and various fragments of the Aβ peptide demonstrate that both parallel and antiparallel β-sheet orientations are observed in amyloid fibrils (Benzinger, 1998; Benzinger, 2000; Gregory, 2000; Antzutkin, 2000; Balbach, 2000; Lansbury, 1995). Indeed, it is not surprising that fibrils made from proteins such as transthyretin and immunoglobulin light chains differ in some structural details from fibrils made from short peptides such as β-amyloid.

The common feature of β-sheet structure in amyloid fibrils, formed by proteins that are otherwise structurally diverse, suggests that peptide backbone hydrogen bonding may be important in the assembly and stability of amyloid fibrils. Kheterpal et al. (2000) recently used hydrogen-deuterium exchange to probe the importance of backbone hydrogen bonding in Aβ1-40 fibrils. These experiments demonstrated that 50% of the backbone hydrogen bonds in Aβ1-40 fibrils resist exchange even after 1,000 h at room temperature. These data suggest that a highly protected, rigid core structure of backbone hydrogen bonds exists in the amyloid fibril. Although this study did not identify the protected residues, there are two distinctly hydrophobic domains in Aβ1-40: the hydrophobic “core domain” between residues 16-22 and the twelve amino acids at the carboxy-terminal of the peptide. It is likely that many of these protected residues are within these two domains.

AD alone is now the fourth-largest killer of adults 65 and older. The disease impacts one of every three families in the United States (Gonzalez-Lima, 1987), and affects over 13 million people world-wide. As the population trends lead to an increase in the number of older people, this number will increase. Thus, it is an important goal of medical science to identify methods of preventing, alleviating or abrogating AD.

The histopathology of AD is characterized by the presence of extracellular plaques and intracellular tangles within the cerebral cortex, hippocampus and the diffuse subcortical projection system. Plaques are made up of a rim of sytrophic neurites surrounding a core of β-amyloid protein formed from abnormally processed amyloid precursor protein (APP). APP is a membrane spanning found in all nerve cells. Tangles occur from an abnormally phosphorylated protein called tau. Duplication of the APP gene is found in trisomy 21 (Down's Syndrome) and leads to an Alzheimer's type pathology in the cerebral cortex of individuals with Down's (Rosser, 1993).

β-amyloid is a 40-43 amino acid proteolytic fragment of the transmembrane APP (Kang, 1987; Goldgaber, 1987; Tanzi, 1987). This protein rapidly associates into insoluble fibrils; in vivo this process is irreversible (Kirschner et al., 1987; Hilbich, 1991; Hilbich et al., 1991; Burdick et al., 1992; Castano et al., 1986). The mechanisms of this aggregation and the structure of the final fibrillar products are not known in detail, however, in this form, the peptides are believed to be neurotoxic. Although these peptides are found in normal brains, they are found at higher concentrations in brains from patients with Alzheimer's disease, and these insoluble fibrils are believed to be pathogenic because they form insoluble plaques and tangles in nerves. Neuritic plaques composed primarily of β-amyloid peptides (Aβ), are widely believed to play a major pathogenic role in Alzheimer's Disease (Selkoe, 1991; Glenner and Wong, 1984; Selkoe, 1994; Geula et al., 1998; Pike et al., 1991; LaFerla et al., 1995; Games et al, 1995; Lambert et al., 1998; Schellenberg, 1995; Hardy, 1997).

It is well recognized in the art that after amyloid deposits have formed, there is no curative treatment which significantly dissolves the deposits in situ (U.S. Pat. No. 5,643,562). Consequently, prevention of β-amyloid aggregation has emerged as a potential goal in the therapy or prevention of Alzheimer's Disease, and similar strategies are possible for related amyloid disorders (Soto, 1999). Such related disorders include, prion disease and Huntington's disease, also dentatorubral pallidoluysian atrophy, spinobulbar atrophy, and several forms of spinocerebellar atrophy. In Huntington's disease, there is selective loss of neurons of the striatum and cortex possible attributable to aggregation of a 250 kDa protein, hungtingtin, which, in people with Huntington's disease, contains polyglutamine expansions of the N-terminal domain. In humans, the disease is associated with polyglutamine expansions of >β40 residues, and the length of the expansion is inversely proportional to the age of onset and directly proportionate to the severity of disease. In transgenic mice, the N-terminal fragment is sufficient to cause a phenotype resembling Huntington's disease, and the ability of the transgenic protein to cause disease depends upon the length the polyglutamine repeat. As with Alzheimer's disease, the exact role of protein aggregation in producing neuronal degeneration is far from certain. Nevertheless, as with Alzheimer's Disease, a potential goal of therapy is to prevent or reverse aggregation of huntingtin, which can be seen within the nucleus and cytoplasm of affected neurons.

Other than amyloidosis there are several other diseases involving fibril formation. For example, prion diseases are characterized by insoluble precipitates or plaques in cells as a consequence of β-fibril formation due to polymerization of the certain prior proteins.

N-methyl amino acids have been used in several systems to control protein and peptide aggregation. An N-methyl amino acid was used to block the dimerization of Interleukin-8 (Rajarathnam et al., 1994). Similarly, N-methyl amino acids have been used to control the aggregation of peptide nanotubes (Clark et al., 1998). Doig (1997) designed a non-aggregating three-stranded β-sheet peptide containing N-methyl amino acids. Recently, Hughes et al., (2000) have applied this strategy in the synthesis of β-sheet (25-35) congeners containing single N-methyl amino acids. In some cases, these peptides ewre found either to alter the morphology or prevent aggregation and neurtoxicity of β-sheet.

N-methyl amino acids have been used in several systems to control, or prevent, the aggregation of β-sheet and β-strand peptides (Chitnumbsub et al., 1999; Rajarathnam et al., 1994; Clark et al., 1998; Hughes et al., 2000; Doig, 1997; Nesloney and Kelly, 1996).

Many investigators have searched for natural inhibitors of fibrillogenesis, or have designed and synthesized inhibitors of Aβ fibrillogenesis. A number of small non-peptide molecules have been shown to inhibit amyloid formation. Nicotine, melatonin, rifampicin and hexadecyl-N-methylpieridinium bromide, for example, block either Aβ aggregation or toxicity. The mechanism of inhibition of these unrelated compounds is not clear, however, and in some cases, high doses of the inhibitor are needed for the effect to be observed.

Peptides homologous to regions of Aβ are also frequently used as inhibitors of fibril formation. Most of these studied have focused on the central hydrophobic “core domain” of Aβ (¹⁷LVFF²¹ A) that is critical for fibrillogenesis. Ghanta et al. and Pallitto et al., for example, designed an inhibitor peptide derived from residues 15-25 that also contains an oligolysine disrupting element. Although this peptide prevented AP toxicity in cell culture it did not block aggregation or fibrillogenesis of Aβ40, and the mechanism by which it blocks toxicity is not certain. Tjernberg et al. reported an acetylated hexapeptide corresponding to this central region that is an effective, equimolar inhibitor of Aβ40 aggregation. A significant problem with this peptide, however, is that it aggregates and forms fibrils by itself. In addition, it has modest solubility is aqueous media, and is susceptible to proteases, both of which could limit its potential as a therapeutic agent. Soto and co-workers have utilized the unique structural properties of the amino acid proline in the design of “β-sheet breaker” peptides derived from the same hydrophobic region, but containing non-conservative amino acid substitutions. Notably, these peptides incorporate prolines into sequences of Aβ fragments, and are reported to be effective inhibitors of fibrillogenesis in vitro and in vivo. Most recently, Hughes, et al. studied congeners of Aβ25-35 that were N-methylated at single residues. Of these, one peptide Aβ25-35, N-methylated at Gly₃₃) blocked the aggregation into fibrils and the toxicity of Aβ25-35. Another peptide (Aβ25-35, N-methylated at Gly₂₅) formed fibrils and was neurotoxic like non-methylated (Aβ25-35), while a third peptide, (Aβ25-35, N-methylated at Leu₃₄), had reduced toxicity and altered fibril morphology but did not eliminate fibril formation by Aβ25-35. Tests of the ability of these singly N-methylated peptides to inhibit fibrillogenesis by full length Aβ40 were not reported.

Other investigators have reported that N-methyl amino acidus induce β-sheet structures in peptides.

Self-association through β-strand domains is required for the physiological activation of certain proteins. For example, replication of the human immunodeficiency virus requires dimerization of an aspartyl protease through β-strand domains of identical subunits. Similarly, interleukin-8 dimerizes through β-strand domains, though it is not certain whether dimerization is required for activity. In all of these cases, whether physiological or pathological, the self-association of proteins through N-strand domains is potentially important in activating the protein in question.

The replacement of amide bonds by ester bonds has been used to investigate the importance of backbone hydrogen bonding (Bramson, 1985; Coombs, 1999; Lu, 1997; Lu, 1999; Arad, 1990; Chapman, 1997; Koh, 1997; Beligere, 2000), since ester bonds, like peptide bonds made using N-methyl amino acids, lack the proton which, in an ordinary peptide, is a potential hydrogen bonding site. At the same time, the ester bond shares many structural similarities with the amide bond, such as a trans conformation and similar bond lengths and angles (Wiberg, 1987; Ingwall, 1974).

Methods and compositions that prevent and/or inhibit the process of fibrillogenesis would improve the treatment, prevention and cure of pathologies that involve formation of fibrils.

### SUMMARY OF INVENTION

The present invention relates generally to fibrillogenesis. More particularly, it provides methods and compositions that inhibit fibril formation and/or promotes disassembly of pre-formed fibrils thereby preventing plaque formation seen in numerous pathologies such as Alzheimer's Disease, prion-mediated diseases, and Huntington's disease. Compositions are provided comprising peptides with short β-strands with two faces: one face is capable of binding to β-amyloids through hydrogen bonds, and the other face blocks propagation of hydrogen bonding needed to form fibrils. Particular aspects of the present invention include the use of such peptide compositions that are short congeners of the fibril proteins containing N-methyl amino acids in alternate positions with or without N-α-acetylated amino acids for the inhibition of fibril formation and for the disassembly of pre-existing or pre-formed fibrils. Other peptide compositions use ester bonds instead of N-methyl amino acids. Specific aspects of the invention address β-amyloid fibrils, prion mediated fibrils, and Huntington protein fibrils.

The present invention overcomes deficiencies in the art by providing compositions and methods that prevent fibrillogenesis. Effective peptide based inhibitors have been created which inhibit fibril formation. In some instances the peptides of the present invention also mediate the disassembly of pre-existing fibrils. Therefore, the invention provides compositions for both preventative and curative therapies of fibril based pathologies.

Cogeners of the hydrophobic “core domain” of Aβ, containing N-methyl amino acids at alternate positions, or ester bonds, are potent inhibitors of full length Aβ fibrillogenesis, and also disassemble pre-formed Aβfibrils. One of the most potent of these inhibitors, termed Aβ16-22m, has the sequence NH₂—KL(me-L)V(me-F)F(me-A)E-CONH₂. In contrast, a peptide NH₂—KL(me-V)(me-F)(me-F)(me-A)-E-CONH₂ with N-methyl amino acids in consecutive order, was a much poorer fibrillogenesis inhibitor. Another peptide containing alternating N-methyl amino acids but based on the sequence of a different fibril-forming protein, the human prion protein, was not an inhibitor of Aβ40 fibrillogenesis. The non-methylated version of the inhibitor peptide, NH₂—KLVFFAE—CONH₂ (Aβ16-22), was a weak fibrillogenesis inhibitor. Aβ16-22m was highly soluble, approximately 20-40 times as soluble at physiological pH and ionic strength as Aβ16-22. Whereas Aβ16-22 was susceptible to cleavage by chymotrypsin, the methylated inhibitor peptide Aβ16-22m was completely resistant to this protease. CD spectroscopy indicated that Aβ16-22m was a β-strand even as a monomer, albeit with an unusual minimum at 226 nm. Size exclusion chromatography shows that Aβ16-22m undergoes a reversible monomer-dimer self-association. In summary, fibrillogenesis inhibitors with alternating N-methyl and non-methylated amino acids appear to act by binding to growth sites of AP nuclei and/or fibrils, and preventing the propagation of hydrogen bonded structures of β-sheet fibrils.

Rationally designed peptide inhibitors of Aβ fibrillogenesis incorporate N-methyl amino acids into alternate positions of a short sequence based on a hydrophobic “core domain” of Aβ, i.e., residues 17-22, known to be critical for Aβ fibrillogenesis. N-methyl amino acids were utilized in the design of these peptides because they were predicted to disrupt the interpeptide hydrogen bonds that promote Aβ fibrillogenesis. In particular, N-methyl amino acids 1) replace an amide proton that normally stabilizes the β-sheet through hydrogen bonds between β-strands 2) introduce steric hindrance between strands in the β-sheet and 3) induces β-strand structure in the peptide itself because of steric constraints. These inhibitors are useful for treatment of diseases associated with fibrillogenesis.

Several N-methyl peptides, based on the hydrophobic core domain of Aβ1-40, both inhibit fibrillogenesis and disassemble pre-formed fibrils. CD and NMR data indicate that two of these peptides containing N-methyl amino acids in alternate positions, Aβ16-22m and Aβ16-20m, are monomeric β-strands in aqueous solutions.

Inhibitors of Aβ¹ fibrillogenesis are homologous to the hydrophobic core domain of Aβ, but contain N-methyl amino acids in alternating positions. These peptides inhibit Aβ1-40 polymerization and also disassemble pre-formed fibrils. The alternating pattern of N-methyl amino acids is critical for inhibition, because a peptide with sequential N-methyl amino acids is a poor inhibitor of Aβ fibrillogenesis. These peptides were designed so that, when they are arrayed as β-strands, they have two distinct faces: an unmodified face with the full complement of functional groups for forming backbone hydrogen bonds, but a second face containing N-methyl groups, in which the replacement of amide protons by N-methyl groups reduces the potential for hydrogen bonding. Two-dimensional NMR and circular dichroic sprectroscopy data to show that a fibrillogenesis inhibitor peptide, Aβ16-20m or Ac—K(Me)LV(Me)FF—NH₂, the intended structure of an extended D-strand. Furthermore, this structure is resistant to denaturation by heat, urea, guanidine or changes of pH from 2.5 to 10.5.

The inhibitor peptides that are aspects of the present invention were designed both as structural probes of forces that stabilize fibrils (e.g., of the roles of hydrogen bonds and side-chain interactions), and as prototypes for a class of therapeutic agents aimed at disrupting β-sheet-containing fibrils. Recent data have suggested that the formation of Aβ fibrils may be partially an intracellular process. Thus, for both of these goals, it might be advantageous for the peptide to be membrane permeable. Despite the hydrophobic composition of Aβ16-20m, it is extremely water soluble; in addition, it is also soluble in a variety of organic solvents. These properties suggested that the peptide might be able to pass spontaneously through phospholipid bilayers, and indeed it is membrane permeable and passes through both natural and artificial phospholipid bilayers, a property that is significant for drug delivery, diagnostics and inhibitory activity.

Two peptides based on the “core domain” of Aβ and containing N-methyl amino acids in alternate positions do indeed strongly inhibit the fibrillogeneiss of full length Aβ40. Moreover, these petides disassemble pre-formed fibrils made of Aβ. In contrast, potent inhibitors with N-methyl amino acids in alternate positions are superior to poor inhibitors of the same basic sequence but containing an equal or greater number of N-methyl amino acids in consecutive positions. Inhibition is sequence specific, and that an N-methyl peptide from another fibrillar protein, the human prion protein, does not inhibit fibrillogenesis of Aβ.

Two small peptides with N-methyl amino acids at alternate positions function as effecitve inhibitors of Aβ40 fibrillogenesis, and furthermore, disassemble pre-formed A040 fibrils. The inhibitor peptides Aβ16-22m and Aβ16-22mR were designed so that a β-strand would be asymmetric, presenting one face which could bind to a fibril, but a second face which would block further binding. N-methyl amino acids were used to form the “blocking face” because the methyl group removes a backbone hydrogen bond interaction between β-strands in a β-sheet. In addition, the N-methyl amino acids are sterically hindered and tend to be restricted in their backbone conformations to the β-sheet geometry. The advantage of alternating N-methyl amino acids shown by the fact that Aβ16-22m(4), a homologous peptide containing four consecutive N-methyl amino acid residues, was a weak inhibitor. PrPm was also not an inhibitor, suggesting that alternate spacing of N-methyl amino acids was not sufficient to form an inhibitor, i.e., there also needs to be sequence homology to the fibril forming peptide.

The Aβ16-22m and Aβ16-22mR peptides fulfill the predicted design requirements for a fibrillogenesis inhibitor. In addition to inhibiting fibrillogenesis, these peptides also cause disassembly of pre-formed Aβ40 birfils. The latter feature is in common with some well studied inhibitors of fibrillogenesis or cystallization (e.g., polymerization of hemoglobin S, calcium oxalate crystallization, among others), and suggests reversibility of many of the steps of Aβ fibrillogenesis.

Aβ16-22m and Aβ6-22mR also possess two other traits of potential importance in the design of therapeutic or preventative agents. First, they are highly soluble in aqueous solutions. This may be surprising in view of the added hydrophobicity attributable to the N-methyl group, and due to the removal of one potential site of hydrogen bonding between the peptide and water. Nevertheless, the N-methyl peptides are 20-40 times more soluble than the unmethylated congeners as both Aβ16-22m and PrPm were also highly soluble in water. Second, Aβ16-22m is highly resistant to proteolytic digestion. Although the unmethylated congener, Aβ16-22m, contains a scissile peptide bond, the methylated peptide was completely resistant to chymotryptic digestion. Protease resistance has been observed for other N-methyl amino acid-containing peptides and may be a general trait.

The two inhibitor peptides exhibited a reversible monomer-oligomer equilibrium. Based on an analysis of size exclusion chromatography, the aggregation number was calculated to be two, i.e., a monomer-dimer equilibrium. Self-aggregation is often associated with an increase of structure for both α-helical and β-strand peptides. In contrast, the two inhibitor peptides adopted a β-strand conformation as both a monomer and oligomer, and there was no increase in sheet content with increasing peptide concentration, as determined by CD spectroscopy. The CD spectra of Aβ16-22m and Aβ16-22mR were most consistent with a β-sheet conformation. The unusual minimum at 226 nm, noted above, has been observed for some other β-sheet peptides.

Both Aβ16-22m and Aβ16-22mR were potent inhibitors of fibrillogenesis, but the former peptide was consistently observed to be the more effective inhibitor. The same rank order was even more apparent for disassembly of pre-formed Aβ40 fibrils. While these data can be accommodated by the assumption of either a parallel or antiparallel orientation of either inhibitor with respect to the Aβ40 peptide, the antiparallel orientation appears somewhat more likely for the more potent of these two inhibitory peptides, Aβ16-22m, since an antiparallel orientation would minimize unfavorable charge interactions between the Lys and Glu side chains of Aβ16-22m and Aβ40.

The Aβ16-22m and Aβ16-22 mR peptides are as effective or more effective than any other inhibitor of fibrillogenesis reported previously; moreover, they are highly effective at disassembling pre-formed fibrils of Aβ. These peptides serve as prototypes of a new class of therapeutic agents for Alzheimer's disease.

Protein-protein interactions are frequently mediated by stable, intermolecular β-sheets. A number of cytokines, such as IL-8 and MCP, and the HIV Protease, for example, dimerize through β-sheet motifs. Evidence also suggests that the macromolecular assemblies of peptides and proteins in amyloid fibrils are stabilized by intermolecular β-sheets. Interfering with the backbone hydrogen bonding of an amyloidgenic peptide (Aβ16-20) by replacing amide bonds with ester bonds prevents the aggregation of the peptide. Ester bonds were incorporated in an alternating fashion so that the peptide presents two unique hydrogen bonding faces when arrayed in an extended, β-strand conformation; one face of the peptide has normal hydrogen bonding capabilities, but the other face is missing amide protons and its ability to hydrogen bond is severely limited. Analytical ultracentrifugation experiments demonstrate that this ester peptide, Aβ16-20e, is predominantly monomeric under solution conditions, unlike the fibril-forming Aβ16-20 peptide. Aβ16-20e also inhibits the aggregation of the Aβ1-40 peptide and disassembles preformed Aβ1-40 fibrils. These results suggest that backbone hydrogen bonding is critical for the assembly of amyloid fibrils.

Provided herein are methods comprising contacting a cell with a polypeptide comprising a β-strand with a first face and a second face, wherein the first face is adapted to bind a fibril forming protein through hydrogen bonds and/or side chain interactions, and the second face is adapted to block propagation of hydrogen bonds. In an embodiment, the polypeptide composition comprises at least two N-methyl amino acids. In another embodiment, the polypeptide composition comprises at least two N-methyl amino acids on the second face of the polypeptide. In some aspects, N-methyl amino acids are not on the first face of the polypeptide. In other aspects there are at least two N-methyl amino acids in alternating positions in the polypeptide.

In other embodiments, the polypeptide further comprises at least one N-α-acetyl amino acid. In particular, the polypeptide has the sequence Ac—K—(me-F)—F—CONH₂.

In an embodiment, the method provides that the polypeptide is at least four amino acids in length. In one aspect of this embodiment, the polypeptide is at least six amino acids in length.

In an embodiment, the polypeptide is adapted to inhibit β-amyloid fibrillogenesis. In some embodiments the polypeptide is adapted to inhibit full length β-amyloid fibrillogenesis.

In an aspect of the invention, the polypeptide comprises a sequence as in or a fragment thereof. The inventors also contemplate using variations of this sequence such that some of the amino acids may be moved around to different positions in the sequence or amino acids may be moved around to different positions in the sequence, or amino acids may be truncated or mutated. For example, the polypeptide has a sequence comprising NH₂—K(me-L)V(me-F)F(me-A)E-CONH₂. In another example, the polypeptide has a sequence comprising NH₂-E(me-L)V(me-F)F(me-A)—K—CONH₂. Yet further, other examples of polypeptides of the present invention include, but are not limited to Ac—NH—K(me-L)V(me-F)F—CONH₂ and Anth-NH—K(me-L)V(me-F)F—CONH₂ wherein Anth refers to anthranilic acid. It is further contemplated that the N-terminal residue may be modified by a variety of chemicals including anthranilic acid or acetyl acid (see Table 1 for examples).

The inventors also contemplate making peptide inhibitors to other portions and domains of the β-amyloid proteins, such as to the C-terminal domain which also contains hydrophobic amino acids, to the linker domain; and to the N-terminal domain. The invention includes all naturally occurring variants of β-amyloid; as well as mutations, such as, conservative mutations to the peptide sequence; variants that have certain amino acids interchanged in the sequence; functionally equivalent proteins; and other similar variations well known to those of skill in the art.

In another embodiment, the polypeptide is adapted to inhibit prion-mediated fibrillogenesis. In an embodiments, the polypeptide has the sequence NH₂-GA(me-A)AAA(me-V)V—CONH₂.

The polypetide may be adapted to inhibit polyglutamine-repeat fibrillogenesis. A specific the polypeptide has the sequence Ac-(Q-(me-Q))₂Q-CONH₂. The [(Q-(me-Q)] unit may be repeated a number of times to alter the polypeptide to synthesize a more robust inhibitor for polygultamine-repeat fibrillogenesis.

In an embodiment, the composition is further defined as comprising a polypeptide with at least two N-methyl amino acids. In one aspect, the least two N-methyl amino acids are on the second face of the polypeptide. In another aspect, there are no N-methyl amino acids on the first face of the polypeptide. In yet another aspect, there are at least two N-methyl amino acids in alternating positions in the polypeptide.

The polypeptide may be adapted to inhibit β-amyloid fibrillogenesis. In another embodiment, the polypeptide is adapted to inhibit full length β-amyloid fibrillogenesis.

Thus, the inventors envision that the polypeptides of this invention can be adapted to inhibit the fibrillogenesis of virtually any fibril forming protein. Therefore, this invention provides polypeptide compositions adapted to inhibit fibrillogenesis of any fibril forming protein.

The invention also provides methods for screening potential fibrillogenesis inhibitors including the following step: a) obtaining a sample containing fibril forming proteins; b) contacting the sample with a composition including a polypeptide comprising a N-strand with a first face and a second face, wherein the first face is adapted to bind a fibril forming protein through hydrogen bonds and/or side chain interactions, and the second face is adapted to block propagation of hydrogen bonds; c) measuring the inhibition of fibril formation; and d) comparing the degree of inhibition to a standard.

A sample is defined herein to include one or more cells, a cellular extract, a cell lysate, a tissue, a tissue extract or lysate, a biopsy sample, a biological fluid, serum, blood.

The invention also provides methods for screening potential fibril dissemblers including the following steps: a) obtaining a sample containing fibrils; b) contacting the sample with a composition including a polypeptide comprising a β-strand with a first face and a second face, wherein the first face is adapted to bind a fibril forming protein through hydrogen bonds and/or side chain interactions, and the second face is adapted to block propagation of hydrogen bonds; c) measuring the disassembly of the protein fibrils; and d) comparing the degree of dissembling to a standard.

The invention also provides methods for detecting fibrils including the steps of: a) contacting a subject with a composition including a polypeptide fibril inhibitor; and b) detecting the presence of fibrils by detecting the binding of the polypeptide to fibrils. Specifically, it is contemplated that the subject is a human that has amyloidosis. In specific aspects, contacting comprises intravenous or oral administration of the inhibitor. Yet further, the inhibitor may be conjugated to a radiolabel or to a radiographic contrasting agent which can be detected by the methods known to this of skill in the art.

For methods of the present invention the cell contacted with the polypeptide including a central nervous system cell, a peripheral nervous system cell, a muscle cell, a pancreas cell, gastrointestinal cell, liver cell and/or heart cell. A suitable cell is a brain cell, in particular a neuron. Those of skill in the art will realize that the use of “a cell” herein, includes a plurality of cells.

The invention contemplates that the method may be performed in vitro as well as in vivo. The method may be assayed in vitro to determine whether a candidate polypeptide inhibits fibrillogenesis and/or disassembles fibrils.

The in vivo applications include methods of inhibiting fibrillogenesis and methods of disassembling fibrils, in particular pre-existing fibrils. The method is useful to prevent the formation of a pathology that requires fibril formation.

The invention further provides that the cell or plurality of cells to which methods and compositions of the present invention are applied is in a subject having a pathological state involving fibril formation. The pathological states that are contemplated to benefit from the therapies provided by the methods are selected from the group consisting of Alzheimer's Disease, Down's Syndrome, Dutch-Type Hereditary Cerebral Hemorrhage Amyloidosis, Reactive Amyloidosis, Familial Mediterranean Fever, Familial Amyloid Nephropathy with Urticaria and Deafness, Muckle-Wells Syndrome, Idiopathic Myeloma, Macroglobulinemia-Associated Myeloma, Familial Amyloid Polyneuropathy, Familial Amyloid Cardiomyopathy, Isolated Cardiac Amyloid, Systemic Senile Amyloidsis, Familial Amyloidotic Polyneuropathy, Scrapie, Creutzfeldt-Jacob Disease, Gerstmann-Straussler-Scheinker Syndrome, Bovine Spongiform Encephalitis, prion-mediated diseases, Huntington's Disease.

The subject treated by the methods described herein generally exhibits amyloidosis. The present invention may be used to treat and/or diagnose a subject that has protein aggregation diseases or protein misfolding diseases. The subject is a mammal. In more specific aspects the subject is a human.

The invention also provides that the methods further comprise administering a pharmaceutical composition comprising a polypeptide of the invention and a pharmaceutically acceptable buffer, solvent or diluent to a subject. In one aspect, the administering is effected by regional delivery of the pharmaceutical composition. In another aspect, the administering comprises delivering the pharmaceutical composition endoscopically, intratracheally, percutaneously, or subcutaneously.

The word “a” and “an,” when used in conjunction with the word comprising, mean “one or more.”

Abbreviations: Aβ, β-amyloid; AD, Alzheimer's Disease; BOC, tert-butoxycarbonyl; CD, circular dichroism; DCC, N,N′-dicyclohexylcarbodiimide; DIC, 1,3-diisopropylcarbodiimide; DMAP, 4-(dimethylamino)-pyridine; DPH, 1,6-diphenyl-1,3,5-hexatriene; FMOC₁₋₉-fluorenylmethoxycarbonyl; HOBt, N-hydroxybenzotriazole; HPLC, high-performance liquid chromatography; HFIP, hexafluoroisopropyl alcohol; IC, inhibitory concentration; MBHA, methylbenzylhydrylamine; TFA, trifluoroacetic acid;

HATU, 2-(1H-9-azabenzotriazol-1-yl)-1,1,3,3-tetramethyluronium hexafluorophosphate; HBTU, 2-(1H-benzotriazole-1-yl)-1,1,3,3-tetramethyluronium hexafluorophosphate; NMR, nuclear magnetic resonance; 2D-NMR, two-dimensional NMR; nOe, nuclear Overhauser effect; ROESY, rotating frame Overhauser spectroscopy; TOCSY, total correlation spectroscopy.

### BRIEF DESCRIPTION OF THE DRAWINGS

FIG. 1(A) is a diagram of Aβ136-22m; (B) is a diagram of Aβ16-22m(4) that illustrates the position of the methyl groups when the peptides are arrayed in a-strand conformation. In FIG. 1(A) and FIG. 1(B), carbon atoms on amide and amino nitrogen atoms are medium gray; other hydrogen atoms are not shown. In Aβ16-22m or Aβ16-22mR, the methyl groups are aligned on only one face of the beta strand. In contrast, the methyl groups are located on both faces of the Aβ16-22m(4) peptide.

FIG. 2(A) shows inhibition of fibrillogenesis and (B) disassembly of Aβ40 fibrils by inhibitor and control peptides. In FIG. 2A, Aβ40 samples were incubated for one week at 37° C. in the presence of various concentrations of peptides; thioflavin induced fluorescence was then measured. In FIG. 2(B), the peptide inhibitors were added to Aβ40 fibrils which had been pre-formed by incubating Aβ40 for one week at 37° C. After addition of peptide inhibitors, the mixtures were incubated for an additional three days at 37° C. After incubations, a 5 μl aliquot of peptide solution was diluted into 1 mil of 50 mm glycine, pH 8.5, containing 5 uM thioflavin. Data are expressed as a percentage of the signal obtained in the absence of inhibitor peptides. Symbols are as follows: (●) Aβ16-22m; (▪) Aβ16-22mR; (Δ) Aβ16-22; (□) Aβ16-22m(4); (x) PrPm; (∘) Ac-Aβ16-22.

FIG. 3(A) shows electron microscopic examination of the effect of Aβ16-22m on fibril formation, electron micrographs of Aβ40 fibrils formed after a one week incubation at pH 7.4. Magnification, × 42,000. (B) is an electron micrograph of Aβ40 incubated with Aβ16-22m (30-fold molar excess) for seven days. Magnification, × 17,000.

FIG. 4(A) shows analytical ultracentrifugation sedimentation equilibrium of 100 μM; (B) 500 μM; and (C) 5 mM solution of Aβ16-22 min buffer (100 mM phosphate, 15mM NaCl, pH 7.4) at 36,000 rpm, 48,000 rpm and 54,000 rpm. The data are displayed as normalized log plots. A homogeneous sample should exhibit a series of parallel lines with the same slope (MW) for all rotor speeds. The solid lines drawn through the data were obtained by fitting the Ln(Absorbance) versus radius² data to an equation of a single ideal species. Higher order fits resulted in poorer agreement with the experimental data. The residual differences between the experimental data and theoretical curves are plotted in the side panels.

FIG. 5 shows circular dichroic spectra of inhibitor peptides. (A) compares the spectra of Aβ16-22 and Aβ16-22m. (13) shows examination of the concentration dependence of the \tilde{β}sheet structure as reflected by the mean residue ellipticity at 226 nm.

FIG. 6 shows results of protease resistance of Aβ16-22 and Aβ16-22m. Peptides were incubated for 24 h at 37 C with 1% (w/v) chymotrypsin. The percentage of undigested peptide was determined by RC-HPLC as described in the Materials and Methods. The data show chromatographs of Aβ16-22m (A) before and (B) after incubation with chymotrypsin; and of Aβ16-22 (C) before and (D) after incubation with chymotrypsin. The arrow marks the position of the intact.

FIG. 7 shows the structure of (A) Aβ16-20m, (B) Anth-Aβ16-20m, (C) Aβ16-20, (D) Aβ16-22R, and (E) PrP115-122m, all arrayed with a β-strand conformation. In all of the N-methylated peptides depicted in the figure, the methyl groups would be aligned on one face of a β-strand.

FIG. 8 shows electron microscopic examination of the effect of Aβ16-20 and Aβ16-20m on Aβ1-40 fibril formation. (A) Electron micrograph of Aβ1-40 incubated in the absence of inhibitor. Magnification, × 17,000. (B) Electron micrograph of Aβ1-40 incubated with a 20-fold molar excess of Aβ16-20m for seven days. Magnification, × 45,000. (C) Electron micrograph of Aβ1-40 incubated with a 20-fold molar excess of Aβ16-20 for seven days. Magnification, × 45,000. (D) Electron micrograph of Aβ16-20 added to Aβ1-40 fibrils which had been pre-formed by incubating Aβ1-40 for five days at 37° C. Magnification × 45,000. (E) Electron micrograph of Aβ16-20 incubated in the absence of other peptides. Magnification × 45,000.

FIG. 9 shows inhibition and disassembly of Aβ40 fibrils by inhibitor peptides. (A) Aβ40 samples were incubated for one week at 37° C. in the presence of various concentrations of peptides; thioflavin fluorescence was measured. In FIG. (B), the peptide inhibitors were added to Aβ40 fibrils which had been pre-formed by incubating Aβ40 for one week at 37° C. After addition of peptide inhibitors, the mixtures were incubated for an additional three days at 37 C. After the incubation, a 10 μl aliquot of peptide solution was diluted into 1 ml of 50 mM glycine, pH 8.5, containing 5 μM thioflavin. Data are expressed as a percentage of the signal obtained in the absence of inhibitor peptides. The data are fit to an equation for a hyperbola parameters divided from nonlinear least squares analysis. Symbols are as follows: (●) Aβ16-20m; (▪) Aβ16-20; (♦) AnthAβ16-20m; (▴) Aβ16-22R.

FIG. 10 shows the rate of Aβ40 fibrils that had been pre-formed by incubating Aβ40 for one week at 37° C. At the specified time points, a 5 μl aliquot of each peptide solution was diluted into 1 mil of 50 mM glycine, pH 8.5, containing 5 μM thioflavin. Data are expressed as a percentage of the signal obtained in the absence of inhibitor peptides. The data are fit to the equation for a first order rate process. Symbols are as follows: (●)Aβ16-20m; A40 molar ratio, 5:1; Aβ16-20m:Aβ40 molar ratio, 10:1; (♦) Aβ16-20m:Aβ40 molar ratio, 20:1; (▴) Aβ16-20m:Aββ40 molar ratio, 30:1; (▾); Aβ16-20m:Aβ40 molar ratio, β40:1.

FIG. 11 shows inhibition of fibrillogenesis and disassembly of pre-formed fibrils is sequence specific. Aβ40 or Prp106-126 was allowed to form fibrils. Each fibril-forming peptide was tested with Aβ16-20m or Prp115-112m. Extent of fibril formation or fibril disassemble was measured using a thioflavin fluorescence assay, as described herein. The X-axis is the ratio (mol:mol) of inhibitor peptide to fibril forming peptide for the various combinations; the Y-axis is the fluorescence expressed as a percentage of fluorescence obtained in the absence of inhibitor peptide. Lines are designated as representing either fibrillogenesis inhibition, or fibril disassembly. Symbols are as follows: (●) Prp115-122 m+PrP106-126, Inhibition; (▪) Aβ16-20 m+PrP106-126, Inhibition; (♦) Aβ16-20 m+PrP106-126, Disassembly; (▴) PrP115-122 m+Aβ40, Inhibition; (▾) PrP115-122 m+Aβ40, Disassembly.

FIG. 12 shows (A) analytical ultracentrifugation sedimentation equilibrium of a 200 EM solution of Aβ16-20m in buffer (100 mM phosphate, 150 mM NaCl, pH 7.4) at 60,000 rpm. The data are displayed as normalized log plots. The solid lines drawn through the data were obtained by fitting the In (Absorbance) versus radius² data to an equation of a single ideal species. Higher order fits resulted in poorer agreement with the experimental data. (B) The residual differences between the experimental data and theoretical curves are plotted in. (C) Size exclusion chromatography of an Aβ16-20m (1 mM) sample incubated at 37° C. for one hour and (D) 72 hours. The column buffer was 100 mM phosphate buffer with 150 mM NaCl, pH 7.4. Absorbance was measured at 220 nm. The column volume is indicated by an arrow.

FIG. 13 shows circular dichroic spectra of Aβ16-20 and Aβ16-20m. (A) compares the spectra of Aβ16-20 (λ) and Aβ16-20m (ν). The effects of (B) peptide concentration, (C) urea and (D) pH on the β-sheet structure of Aβ16-20m, as reflected by the mean residue ellipticity at 226 nm, are displayed in the following panels. Data were collected as described in the experimental section.

FIG. 14 shows NMR spectroscopy of the Aβ16-20m peptide in phosphate buffer. (A) TOCSY spectra expanded in the Hα proton region. Spin systems are identified by the single letter amino acid code and residue number. (B) ROESY spectra expanded in the Hα proton region. Data were collected on a Varian 600 MHz instrument using presaturation for solvent suppression. Peaks were assigned using the TOCSY and DQF-COSY data.

FIG. 15 shows (A) efflux of (▴) ¹⁴C-Aβ16-20m alone, (♦) ³H-glycine alone, and a mixture of ¹⁴C-Aβ16-20m (▪) and ³H-glycine (●) from phosphotidylcholine vesicles. Phosphtidylcholine vesicles were prepared in the presence of 14C-labeled β16-20, ³H-glycine or a mixture of the two compounds. Free β16-20m and glycine were separated from the vesicles by passage over a G25 column (Pharmacia). The efflux of Aβ16-20m and glycine were measured using an ultrafiltration assay. Flux is expressed as a fraction of the total label; data were fit to a first-order rate equation. Efflux of calcein from phospotidylcholine vesicles. (B) different concentrations of Aβ16-20m (●) and Aβ16-20 (▪) were incubated with phosphotidylcholine vesicles containing calcein for 3 hours at 37° C. The fluorescence of the samples were then measured with an excitation wavelength of 490 nm and an emission wavelength of 520 nm. Data are expressed as a fraction of maximal fluorescence. (C) the rate of calcein efflux from phospotidylcholine vesicles was measured in the presence of β400 μM Aβ16-20m. Fluorescence is expressed in arbitrary units. Data are fit to an equation for a first order rate process. (D) right angle light scattering of a vesicle solution in the presence (▪) or absence (●) of Aβ16-20m. The turbidity of the solutions were measured by following the 90° light scattering on a fluorescence spectrophotometer with both the excitation and emission wavelengths set to 600 nm. (E) and (F) Fluorescence data are expressed as arbitrary units. Fluorescence microscopy of COS cells incubated for twelve hours with 50 μg of Anthβ16-20m. After the incubation period, the cells were washed, fixed with formaldehyde and examined by fluorescence microscopy using a DAPI filter.

FIG. 16 shows structures of (A) Aβ16-20m, (B) Aβ16-20 m2, (C) Anth-Aβ16-20m, (D) Aβ16-20, (E) Aβ16-20s and (F) PrP115-122m. All peptides are displayed in a β-strand conformation. In the N-methyl peptides shown in the FIG., the methyl groups are aligned on one hydrogen bonding face of the D-strand.

FIG. 17 shows Inhibition and disassembly of Aβ1-40 fibrils by inhibitor peptides. In (A), Aβ1-40 samples were incubated for one week at 37° C. in the presence of various concentrations of inhibitor peptides; Thioflavin T fluorescence was then measured as described in Methods and Materials. In (B), the peptide inhibitors were added to Aβ1-40 fibrils which had been pre-formed by incubating Aβ1-40 for five days at 37° C. After addition of the peptide inhibitors, the mixtures were incubated for an additional three days at 37° C. and then the Thioflavin T fluorescence of the samples were measured as described in the experimental section. Data are expressed as a percentage of the signal obtained in the absence of inhibitor peptides. The data were fit to an equation for a hyperbola, as described in the Materials and Methods; parameters are derived from nonlinear least squares analysis. Symbols are as follows: (λ) Aβ16-20m; (ν) Aβ16-20; (υ) Anth-Aβ16-20m; (σ) Aβ16-20 m2; (τ)A, 16-20s.

FIG. 18 shows (A) Equilibrium analytical ultracentrifugation of a 1 mM solution of Aβ16-20m in buffer (100 mM phosphate, 150 mM NaCl, pH 7.4) at 36,000 (λ), 42,000 (ν) and 48,000 (υ) rpm. The data are displayed as normalized log plots. The solid lines drawn through the data were obtained by fitting the In (Absorbance) versus radius² data to an equation of a single ideal species. Higher order fits resulted in poorer agreement with the experimental data. The residual differences between the experimental data and theoretical curves are plotted in (B).

FIG. 19 shows (A) Efflux of (σ) ¹⁴C-Aβ16-20m alone, (υ) ³H-glycine alone, and a mixture of ¹⁴C-Aβ16-20m (v) and ³H-glyine (λ) from phosphatidylcholine vesicles. Phosphatidylcholine vesicles were prepared in the presence of ¹⁴C-labeled Aβ16-20m, ³H-glycine or a mixture of the two compounds. Free Aβ16-20m and glycine were separated from the vesicles by passage over a PD-10 Sephadex G25 column (Pharmacia). The efflux of Aβ16-20m and glycine was measured using an ultrafiltration assay described in the Materials and Methods and quantitated with scintillation counting. Efflux is expressed as a fraction of the total. (B) Efflux of calcein from phosphatidylcholine vesicles. Different concentrations of Aβ16-20m (λ) and Aβ16-20 (ν) were incubated with phosphatidylcholine vesicles containing calcein for 3 hours at 37° C. The fluorescence of the samples was then measured with an excitation wavelength of 490 nm and an emission wavelength of 520 nm. Data are expressed as a fraction of maximal fluorescence. (C) Right angle light scattering of a vesicle solution in the presence (ν) or absence (λ) of Aβ16-20m. The turbidity of the solutions was measured by following the 90° light scattering on a fluorescence spectrophotometer with both the excitation and emission wavelengths set to 600 nm. Scattering data are expressed as arbitrary fluorescence units.

FIG. 20 shows (A) Fluorescence microscopy of COS cells incubated for twelve hours with 40 μM Anth-Aβ16-20m. After the incubation period, the cells were washed, fixed with formaldehyde and examined by fluorescence microscopy using a DAPI filter. (B) HPLC chromatogram of the Anth-Aβ16-20m peptide before incubation with COS cells. The elution gradient was from 0%-60% acetonitrile in 60 minutes. The peptide was detected by measuring the absorbance at 346 nm. (C) HPLC chromatogram of Anth-Aβ16-20m peptide that had been internalized by COS cells and then reisolated, as described in the Materials and Materials. The N-methyl anthranilic acid-labeled peptide was identified in the presence of other cellular peptides and proteins by fluorescence spectroscopy. The excitation and emission wavelengths were 346 nm and 435 nm, respectively. The HPLC gradient is the same as in (A).

FIG. 21 shows that as described above (FIG. 3), Aβ1-40 or Prp106-126 was allowed to form fibrils, as described in Methods, either in the presence of absence of a fibrillogenesis inhibitor. Each fibril-forming peptide was tested with Aβ16-20m or Prp115-122m. Extent of fibril formation or fibril disassembly was measured using a thioflavin fluorescence assay, as described above. In the FIG., the x-axis is the ratio (mol:mol) of inhibitor peptide to fibril forming peptide for the various combinations; the y-axis is the fluorescence expressed as a percentage of fluorescence obtained in the absence of inhibitor peptide. Symbols are as follows: (λ) PrP115-122 m+PrP106-126, Inhibition; (ν) Aβ16-20 m+PrP106-126, Inhibition; (υ) Aβ16-20 m+PrP106-126, Disassembly; (σ) PrP115-122 m+Aβ1-40, Inhibition; (τ) PrP115-122 m+Aβ1-40, Disassembly. (μ) Aβ16-20s+Aβ1-40, Inhibition; (□)Aβ16-20s+Aβ1-40, Disassembly.

FIG. 22 shows inhibition of fibrillogenesis (A) and dissasembly (B) of Aβ40 fibrils by inhibitor and control peptides. Data were collected as described in the experimental section. Data are expressed as a percentage of the signal obtained in the absence of inhibitor peptides. In the figures, points represent experimental data, and the line is a theoretical curve. Data were analyzed on the model of acomplex betweenAβ40 and the smaller peptides, using the equation:

$$\% Fluorescence = 100 \% - ( \frac{{S}_{t} P}{{K}_{d} + P} )$$

where Sₜ is apparent sites of complexation between Aβ40 and the peptide, P is the inhibitor peptide concentration, and K_{d} is the appparent dissociation constant of the Aβ peptide complex. No theoretical curve is provided for the Aβ16-22m(4) peptide because the data did not fit the above equation.

FIG. 23 shows the concentration dependence of the aggregation is analyzed by ploting fraction of oligomer versus total peptide concentration, using the equation in the text.

FIG. 24 shows how size exclusion chromatographs were obtained using a Superdex Peptide (Pharmacia) column. Peptide concentrations were 0.1, 0.2, 0.5, 1.0, 2.0, 3.0, 4.0, 6.0 mg/ml, as indicated. Chromatpographs are scaled so that, in each case, the largest peak is full scale. The data are consistent with the proposal that the ppetides undergo a reversible monomer-oligomer equilibrium. Both peaks eluted after the inclusion volume of the column as determined by the elution time of acetic acid and other low molecular weight markers). Although the recovery of the peptide from the column was virtually quantitative, the late elution of the peptides was consistent with adsorption of the peptide on all of the columns. For this reason, it was not possible to determine a molecular weight of the oligomer by this technique. Nevertheless, the concentration dependency of the aggregation could be analyzed using the following inferences. First, because the relative proportion of peptide in the earlier eluting peak increased with increasing concentration, we inferred that the earlier eluting peak was the oligomer. Second, since no other peaks were ever observed in any of the chromatograms, we inferred that the equilibrium could be analyzed as a simple case involving only two species. Third, because no peptide was observed to elute between the two peaks, and there was no “tailing” or either peak, we inferred that the equilibration was sufficiently slow that significant re-equilibration did not occur within the time frame of the chromatography. Using these inferences, the monomer-oligomer equilibrium was analyzed as described in the text.

FIG. 25 shows the mean residue ellipticity of Aβ16-22m and Aβ16-22mR were independent of peptide concentration. The graph shows the mean residue ellipticity at 226 nm as a function of total peptide concentration.

FIG. 26 shows structures of Aβ16-20 (A), Aβ16-20e (B) Aβ16-20m (C) PrP117-121 e (D) and (E) Aβ16-20-Bpa drawn with the peptide in β-strand conformationss. In the ester and N-methyl peptides, the backbone modifications at alternating residues are aligned on one hydrogen bonding face of the β-strand.

FIG. 27 ESI-MS detects non-covalent dimers of the Aβ peptides. Shown are ESI-MS spectra of 250 μM solutions of Aβ16-20e (A), Aβ16-20 (B) and Aβ16-20m (C). The samples were prepared in deionized water and the data were collected as described in the Materials and Methods section. The peaks corresponding to the monomer and dimer molecular weights for each peptide are labeled on the spectra.

FIG. 28 shows Aβ16-20-Bpa forms a covalent dimer upon irradiation with UV light. The MADI-MS spectrum of a 500 μM solution of Aβ16-20 Bpa irradiated for 30 min at 350 nm shows peaks at 801.1 Da and 1600.8 Da, corresponding to monomeric and dimeric A 16-20-Bpa, respectively. The inset panel demosntrates that in the absence of irradiation, the dimer peak at 1600.8 Da is not observed in the MALDI-MS spectrum.

FIG. 29 shows Aβ16-20 Bpa is crosslinked to Aβ31-40 upon irradiation with UV light. (A) shows SDS-PAGE gel analysis of a mixture of Aβ16-20-Bpa and Aβ1-40 that was incubated in the absence (lane 1) or presence (lanes 2, 3, 4, 5, and 6) of near-UV light for different amounts of time. (B) shows MALDI-MS analysis of the Aβ16-20-Bpa and Aβ1-40 mixutre after exposure to near-UV light. The peak at 4331.05 Da represents the monomeric Aβ1-40 peptide. The peaks at 5133.24 Da and 5936.27 Da correspond to Aβ1-40 crosslinked to one and two Aβ16-20-Bpa peptides, respectively.

### DESCRIPTION OF ILLUSTRATIVE EMBODIMENTS

A. The Present Invention

The present inventors have designed, synthesized, and biochemically characterized polypeptide inhibitors of fibrillogensis. These polypeptides comprise short β-strands with two faces: one that can bind to β-amyloid through hydrogen bonds, and one which blocks propagation of hydrogen bonding needed to form fibrils. In some embodiments, these polypeptides comprise N-methyl amino acids with or without N-α-acetylated amino acids. In other embodiments ester bonds serve to block or dissemble fibrillogenesis. In general, these polypeptides are based on the sequence of the hydrophobic “core domain” of β-amyloid, i.e., residues known to be critical for β-amyloid fibrillogenesis (Lansbury, 1997; Harper and Lansbury, 1997; Rochet and Lansbury, 2000; Benzinger et al., 1998; Gregory et al., 1998; Benzinger et al., 2000). The invention also provides other such polypeptides based on the sequence of prion proteins and polyglutamine repeat proteins. The inventors contemplate fibrillogenesis inhibitor and disassembler polypeptides based on the sequence of any fibril forming protein.

N-methyl amino acids are utilized in the design of these peptides because they disrupt the interpeptide hydrogen bonds that promote fibrillogenesis. In the example of β-amyloid fibrillogenesis, the N-methyl groups prevent intermolecular association by a combination of effects. First, they eliminate hydrogen bonding on one “face” of a β-strand structure. Second, they interact with a specific target not only through hydrogen bonding on one “face” of a β-strand; but also through specific side chain interactions. Third, they are conformationally rigid, and serve as pre-formed or pre-structured β-strands to which a specific β-sheet-forming partner can conform. That is, N-methyl amino acids introduces a rigidity to peptides that severely reduces the entropy of the inhibitors compared to non-methylated congeners, and thereby facilitates association of its target partner. Fourth, the inhibitor peptides are twisted or distorted β-strands, which prevents them from self-associating as dimers and limits the size of inhibitor-target complexes, probably to a 1:1 stoichiometric complex in most cases. Finally, they have “amphibian” solubility properties, which renders them highly soluble in aqueous media, but also permeable to cell membranes and synthetic phospholipid bilayers. The cause of the water solubility is unknown. In the case of Aβ16-20m, one might make an analogy to an ionic detergent, in which event a single charge can render the detergent molecule water-soluble. In the case of Aβ16-20m, there is a single positive charge on the lysine side chain. However, Aβ16-22m is also extremely water soluble but is zwitterionic, and Prp115-122m has no charges at all and is the most water soluble of these peptides. At the same time, these peptides are able to pass through lipid bilayers and dissolve in organic solvents such as DMF, methylene chloride, or chloroform.

Therefore, the invention provides polypeptide sequences, based on the sequence of the “core domain” of β-amyloid and containing N-methyl amino acids in alternate positions that strongly inhibit the fibrillogenesis of full-length β-amyloid (Aβ) β40. The “core domain” is the domain of the protein known to be critical for Aβ fibrillogenesis, i.e., amino acid residues 15-22. Examples of the polypeptide sequences that are contemplated in the present invention include, but are not limited to (Aβ16-22): NH₂—KLVFFAE—CONH₂; (Aβ16-22): NH₂—K(me-L)V(me-F)F(me-A)-E-CONH₂; (Aβ16-22mR): NH₂-E(me-L)V(me-F)F(me-A)—K—CONH₂; (APB16-22M(4)); NH₂—KL(me-V)(me-F)(me-F)(Me-A)-E-CONH₂; (Aβ16-20m): Ac—NH—K(me-L)V(me-F)F—CONH₂; (Anth Aβ16-20m): Anth-NH—K(me-L)V(me-F)F—CONH₂; (Aβ16-20R): Ac—NH—KL_{red}VF_{red}F—CONH₂; (Aβ16-20: EAc—NH—KLₑₛₜₑᵣVFₑₛₜₑᵣF—CONH₂; (Ac Aβ16-22): AcNH—KLVFF—CONH₂; and (Aβ1-40: NH₂-DAEFRHDSGYEVHHQKLVFFAEDVGSNKGAIIGLMVGGVVIA-COOH). One of skill in the art is aware that “red” refers to reduced and “ester” refers to esterified.

It is also envisioned that the above sequences may be further modified to alter polypeptide to synthesize a more robust inhibitor. Such modifications are described herein and well known in the art.

Furthermore, the invention also provides that these polypeptides disassemble pre-formed fibrils made of β-amyloid.

Several peptides containing N-methyl amino acids inhibit fibrillogenesis and promote disassembly of amyloid fibrils. All of the peptides exhibit IC₅₀ values at molar ratios of inhibitor to Aβ1-40 in the range of 2-10. Although the N-methyl amino acids need to be in alternate positions, the specific placement of the methyl groups does not appear to be significant; Aβ16-20m, with N-methyl groups at residues 17 and 19, and Aβ16-20 m2, methylated at residues 18 and 20, exhibit similar fibril inhibition and disassembly properties. The N-methyl peptides, Aβ16-20m and Aβ16-20 m2, are more effective at inhibiting fibrillogenesis and disassembling fibrils than the non-methylated peptide, Aβ16-20. The Anth-Aβ16-20m peptide is even more effective in inhibiting Aβ1-40 fibrillogenesis and disassembling fibrils than the Aβ16-20m peptide. The cause of the increased efficacy of Anth-Aβ16-20m over Aβ16-20m is not known.

Circular dichroism and one- and two-dimensional NMR data show that the structure of Aβ16-20m is most consistent with the intended β-strand conformation. By the criterion of CD spectra, this β-strand conformation is remarkably insensitive to solvent conditions. The CD spectra are invariant over a pH range of 2.5 to 10.5 and urea concentrations of 0 to 8 M. Unlike the methylated Aβ16-20m peptide, Aβ16-20 exhibits a random coil CD spectrum. Thus, Aβ16-20m possesses an unusual degree of conformational rigidity. It is possible that the structural stability of N-methyl peptides may contribute, among other factors, to their inhibitory properties. Solid state NMR work has shown that the central, hydrophobic domain of Aβ1-40, encompassing residues 16-20, adopts an extended β-strand structure in the amyloid fibril (Benzinger et al., 1998, 2000; Gregory et al., 1998; Balbach et al., 2000; Antzutkin et al., 2001). Since the N-methyl peptides are constrained to a N-strand conformation these peptides may be preorganized for interacting with Aβ1-40. Preorganization of Aβ16-20m into its Aβ1-40-bound conformation may reduce the entropic barrier on the route to the inhibitor-Aβ1-40 complex. Cyclic inhibitors of the HIV protease, for example, are 10-100 times more effective than acyclic analogues due to reduced conformational entropy. Most of the entropy gain of HIV inhibitors, however, arises from the desolvation of hydrophobic groups. A similar desolvation effect due to the release of water molecules from the hydrophobic Aβ16-20m peptide upon binding Aβ1-40, consequently, may also contribute favorably to the entropy of the binding process. Without a more detailed analysis of the interaction between Aβ16-20m and Aβ1-40, it is difficult to predict the different entropic and enthalpic contributions to binding.

The CD spectra also suggest that the N-strand structure may be twisted or distorted. While the CD spectrum has a single minimum that is most consistent with a β-strand structure, the minimum is red-shifted to 226 nm. This shift has been observed with other β-sheet peptides and is often attributed to the twist of the strand. In addition, a similar red-shift was also observed in the spectra of other peptides containing N-methyl amino acids.

A notable trait of Aβ16-20m, and, indeed, the other N-methyl inhibitors (Aβ16-22m, Aβ16-20 m2, Aβ16-20s and PrP115-122m), is their high solubility in water. This trait is especially striking in view of the amino acid composition of these peptides. In the case of Aβ16-20m, four of the five amino acids are hydrophobic, both the amino and carboxyl termini are blocked, and two of the potential hydrogen bonding sites in the peptide backbone are methylated. Despite this composition, the peptide was soluble in aqueous media at ≧30 mM. This is in striking contrast to the non-methylated peptide, Aβ16-20, which is only sparingly soluble (≈1 mM) at neutral pH and physiological salt concentrations, and which self-associates and forms fibrils in solution. Aβ16-20m, on the other hand, yields a monomeric molecular weight by analytical ultracentrifugation, and shows no evidence of self-association. Indeed, no evidence for self-association has been observed for any of the N-methyl peptides. CD and NMR data also support this contention. The mean residue ellipticity of Aβ16-20m is constant over a concentration range of 0.01 mM to 11 mM, and no evidence of line broadening was observed in NMR spectra performed on peptide samples over a similar concentration range. The cause of the water solubility is not obvious. In the case of Aβ16-20m, which has a single positive charge on its lysine side chain, one might make an analogy to an ionic detergent, in which even a single charge can render the detergent molecule water-soluble. However, Aβ16-22m is zwitterionic and also extremely water-soluble. In addition, PrP115-122m has no charges at all and is the most water soluble of the N-methyl peptides. Both the water solubility and the monomeric state Aβ16-20m may be attributable in part to the fact that, while this peptide retains some functional groups that enable it to form hydrogen bonds with water, the distortion of the β-strand prevents it from self-associating, and precludes even the formation of Aβ16-20m dimers. Since Aβ16-20m retains one “normal” hydrogen bonding face as a N-strand, the question then arises how Aβ16-20m can interact with Aβ1-40 and inhibit its fibrillogenesis, but is not able to dimerize with itself. One possible explanation is that the relatively flexible Aβ1-40 peptide, unlike the conformationally rigid Aβ16-20m peptide, may be able to adjust to the backbone hydrogen bonding pattern of Aβ16-20m and, thereby, facilitate the formation of an inhibitor-Aβ1-40 complex, while two or more molecules of Aβ16-20m are too rigid to conform to each other and form an aggregate.

Aβ16-20m was found to be highly soluble not only in aqueous media but also in organic solvents such as dimethylformamide, dichloromethane, and even diethyl ether. The high solubility of Aβ16-20m in both aqueous and organic solvents is a property shared by certain hydrophilic polymers, such as polyethylene glycol. The inhibitor peptides have mainly hydrophobic amino acid side chains and the N-methyl groups would seem likely to increase the lipophilicity of the peptides. Indeed, N-methyl amino acids have been used in other studies to increase the lipophilicity and membrane permeability of small peptides. ³H-glycine, which by itself passes out of vesicles at a slow rate, rapidly effluxes from vesicles when it is placed in the included volume of the vesicle along with Aβ16-20m. The data on ³H-glycine efflux are also consistent with the data demonstrating increases in calcein fluorescence as peptide leaks out of the vesicles. Similarly, the Anth-Aβ16-20m peptide passes readily into COS cells without any morphological disruption of these cells. These inhibitor peptides, consequently, may also be able to pass effectively through the blood-brain-barrier. As controls, Anth-Aβ16-20 does not permeate into COS cells. This observation is consistent with the idea that the N-methyl groups are necessary for Anth-Aβ16-20m to pass into cells; in addition, the N-methyl anthranilic acid group does not confer the ability of Anth-Aβ16-20m to pass into cells, since Anth-Aβ16-20 did not pass into cells. Furthermore, N-methyl amino acids do not appear to be sufficient to allow Anth-Aβ16-20m to pass into cells, as the Anth-PrP115-122m does not permeate into the same (COS) cells. There are two manifest differences between Anth-Aβ16-20m and Anth-PrP115-122m: the former peptide is more hydrophobic than the latter, and the former peptide has a sole charged residue and a net charge of +1, while the latter has no charges at all.

The exact mechanism by which Aβ16-20m passes through phospholipid bilayers is uncertain. Light scattering data suggest that the peptide does not cause fusion of the vesicles, however, and microscopy does not indicate any gross abnormality of the cells into which Anth-Aβ16-20m has entered. Curve fitting of the efflux of ¹⁴C-Aβ16-20m from lecithin single bilayer vesicles suggests that all of the label can be lost from the included volume. Although the data do not exclude the possibility that a minute fraction of peptide is retained indefinitely within the bilayer, the lack of obvious alteration of vesicles or cells by Aβ16-20m or Anth-Aβ16-20m, respectively, suggests that the peptide does not make permanent channels in the vesicles.

These inhibitor peptides exhibit a degree of sequence specificity. An N-methyl peptide based on the prion protein, PrP115-122m, does not inhibit Aβ1-40 aggregation. PrP115-122m, however, does inhibit the fibrillogenesis of PrP106-126, derived from the prion protein. Conversely, Aβ16-20m did not inhibit fibrillogenesis of PrP106-126. This amino acids sequence specificity is not absolute, however. A scrambled version of Aβ16-20m, Aβ16-20s, is an effective inhibitor of Aβ1-40 fibrillogenesis. While it is not possible to scramble this short and somewhat redundant sequence very much, the efficacy of Aβ16-20s as a fibrillogenesis inhibitor suggests that while a degree of sequence homology is necessary for the interaction between an inhibitor and Aβ1-40, amino acid composition may be as important as the exact amino acid sequence. Aβ16-20m is composed of primarily large, hydrophobic amino acids, while PrP115-122m is composed of predominantly alanine residues.

The membrane permeability of Aβ16-20m and Anth-Aβ16-20m may be an important advantage of the N-methyl strategy of disrupting peptide-peptide and protein-protein interactions through β-sheets, since the blood-brain-barrier and cellular membranes are impermeable to most peptides. Recent evidence suggests that the oligomerization of Aβ may begin intracellularly. Intracellular Aβ dimers were detected in both neuronal and nonneuronal cell lines. Inhibition of fibrillogenesis, consequently, may require membrane permeable molecules. The invariance of the CD spectrum of Aβ16-20m over a pH range of 2.5-10.5 suggests that Aβ16-20m may function as an inhibitor even in acidic cellular compartments, such as the endosome, where amyloid fibrillogenesis has been hypothesized to occur. In addition, the lipophilicity of the N-methyl peptides also suggest that they may be used as diagnostic agents. Although there are a number of methods for staining fibrils in post-mortem tissue sections, there are currently no methods for detection of amyloid accumulation in vivo. A recent study, however, did report the design of a probe, BSB, that labels Aβ aggregates in vivo in a mouse model of Alzheimer's Disease. BSB is not specific for fibrils composed of the β-amyloid peptide; this probe also binds to neurofibrillary tangles (AD), Lewy bodies (Parkinson's Disease) and glial cell bodies (multiple-system atrophy). The N-methyl inhibitor peptides exhibit a degree of sequence specificity, which suggests that a radiolabeled Aβ16-20m peptide may potentially function as an in vivo, diagnostic tool specific for Alzheimer's Disease.

The characterization of the properties of the N-methyl amino acid-containing inhibitors of peptide and protein aggregation may allow for a more general approach to this problem not only in fibril-forming proteins such as β-amyloid, huntingtin, and the prion protein, but also in systems as diverse as the HIV protease and chemokines, in which there is dimerization through β-strand domains.

In other examples, the invention provides polypeptides that inhibit fibrillogenesis and/or also disassemble pre-formed fibrils for sequences based on prion proteins, for example, the polypeptide having the sequence NH₂-GA(me-A)AAA(me-V)V—CONH₂ and Huntington's proteins, for example, polypeptides based on the polyglutamine repeat sequences. Examples of these polypeptide sequences include, (prion peptide) NH₂—KTNMKHMAGAAAAGAVVGGLG-COOH; and (Huntingtin inhibitor) Ac-(Q-(me-Q))₂Q-CONH₂.

Thus, the invention provides polypeptides and peptides that are potent inhibitors of fibrillogenesis and/or dissassemblers of pre-formed fibrils which may comprise N-methyl amino acids with or without N—acetyl amino acids. In another embodiment the N-methylated amino-acids are at alternate positions. In some specific aspects the N—acetylated amino acid is at the N-terminal of the protein. In other aspects some inhibitors comprise three or four N-methyl amino acids.

The polypeptides have sequence specificity with respect to inhibition of fibril formation and/or fibril disassembly, for example, while an N-methyl peptide from a fibrillar protein such as the human prion protein, inhibits prion protein fibril formation it does not inhibit fibrillogenesis of O-amyloid and vice versa. Therefore, the invention provides peptides that inhibit a wide variety of fibril formations and/or fibril disassembly. In some non-limiting examples, the polypeptides of the invention can inhibit and/or disassemble fibrils such as. β-amyloid fibrils; prion protein fibrils; fibrils involved in Huntington's disease containing the polyglutamine repeats; β-amyloid fibrils; light chain fibrils.

The invention also explains mechanisms that govern the fibril inhibition and/or fibril disassembly.

N-methyl amino acids have been used in several systems to control protein and peptide aggregation. For example, an N-methyl amino acid was used to block the dimerization of Interleukin-8 (Rajarathnam et al., 1994). Similarly, N-methyl amino acids have been used to control the aggregation of peptide nanotubes (Clark et al., 1998). Doig (1997) designed a non-aggregating three-stranded β-sheet peptide containing N-methyl amino acids. Recently, Hughes et al. (2000) have applied this strategy in the synthesis of β-amyloid congeners containing single N-methyl amino acids. In some cases, these peptides were found either to alter the morphology or prevent aggregation and neurotoxicity of β-amyloid.

The inventors also provide polypeptides that are adapted to inhibit prion-protein sequence and are not limited to the specific polypeptide sequence described herein. Another example provided herein is polypeptides that are adapted to inhibit and/or disassemble polyglutamine fibril formation.

The replacement of an amide bond with an ester bond is an established method for investigating the role of backbone hydrogen bonding. The ester group is a conservative substitution for the amide group because both the ester and amide bond adopt predominantly a trans, planar conformation and share similar Ramachandran plots (Wiberg, 1987; Ingwall, 1974; Ramakrishnan, 1978). The primary difference between the amide and ester bond is that the hydrogen bond donating amide-NH is replaced with an electronegative oxygen atom. In addition, the ester carbonyl is less basic than the amide carbonyl and, as a consequence, is a weaker hydrogen bond acceptor (Arnett, 1974).

This strategy of replacing amide bonds with ester bonds has been employed in a number of studies investigating both intramolecular and intermolecular hydrogen bonding interactions (Bramson, 1985; Coombs, 1999; Lu, 1997; Lu, 1999; Arad, 1990; Chapman, 1997; Koh, 1997; Beligere, 2000). Lu et al., for example, used an amide-to-ester replacement to investigate an intermolecular hydrogen bond stabilizing a protease-inhibitor complex (Lu 1999; Lu 2000). Similarly, Schultz et al. utilized ester bonds to probe hydrogen bonding in both α-helix and β-sheet secondary structures (Koh, 1997; Chapman, 1997). Recently, Beligere et al. replaced four amide bonds that span the length of a helix in chymotrypsin inhibitor 2 with ester bonds and demonstrated that the protein folds into a functional, although destabilized, structure (Beligere, 2000).

Thus, the Aβ16-20e peptide was compared to both the unmodified congener Aβ16-20 and the inhibitor peptide Aβ16-20m for its ability to inhibit Aβ1-40 fibrillogenesis and disassemble pre-formed Aβ1-40 fibrils. All three peptides inhibit fibrillogenesis and disassemble pre-formed fibrils; the efficacy of Aβ16-20e is similar to that of Aβ16-20m, both of which are better inhibitors than Aβ16-20. Aβ16-20, though an inhibitor of fibrillogenesis, resembles its parent peptide, Aβ1-40, in that it forms fibrils by itself. The Aβ16-20 fibrils appear by electron microscopy as long, unbranched amyloid fibrils and cause the typical redshift in the spectrum of Congo Red dye. These fibrils do not induce thioflavin T fluorescence, however, a trait shared by other short amyloidogenic peptides. In contrast to Aβ16-20, neither Aβ16-20m nor Aβ16-20e form fibrils, as shown by electron microscopy, and by thioflavin T and Congo Red binding assays.

A molecular weight of approximately 730 Da was obtained for Aβ16-20e by analytical ultracentrifugation, which demonstrates that this peptide is predominantly monomeric in solution. A disadvantage of analytical ultracentrifugation, however, is that it is often difficult to identify weakly aggregating species, particularly for low molecular weight peptides (Cole, 1999). A small amount of a dimeric peptide in the presence of predominantly monomeric peptide, for example, is not readily identifiable by analytical ultracentrifugation.

In recent years, ESI-MS has emerged as a powerful technique for studying weak, non-covalent interactions between proteins or between proteins and other ligands (Pramanik 1998; Baca 1992; Hsieh 1995; Li 1993). Unlike other techniques, such as analytical ultracentrifugation and size exclusion chromatography, ESI mass spectrometry provides the exact molecular weight of a complex, even in the presence of high concentrations of other species. In electrospray ionization, charged droplets are generated at atmospheric pressure by spraying a sample under a strong electric field. This ionization process is very “soft” and leaves the ions largely unfragmented, which facilitates the observation of non-covalent complexes. Chen et al (1997), for example, used ESI-MS to investigate the conformation and aggregation of the Aβ1-40 peptide. In these experiments, monomeric, dimeric, trimeric and tetrameric Aβ1-40 species were observed by ESI-MS.

ESI-MS analysis of A 16-20 and Aβ16-20e demonstrate that both of these peptides form dimers in solution. The crosslinking results for the Aβ16-20-Bpa peptide is consistent with both the AUC and ESI-MS data because it demonstrates that Aβ16-20e forms a small amount of a dimeric species in solution, which is not readily detectable by analytical ultracentrifugation. The N-methyl peptide does not appear to form a dimer to nearly the same extent as Aβ16-20 or Aβ16-20e. This is consistent with the recent report of a pentapeptide containing two alternating N-methyl amino acids that exhibits a K_{d}>150 mM for dimerization (Phillips, 2001).

These observations are also consistent with the observation that Aβ16-20m and other N-methylated peptides form distorted or twisted β-strands, which severely hinders the formation of dimers. Aβ16-20e, in contrast, can form a dimer, albeit at high concentrations. The high concentration of Aβ16-20e needed for dimerization indicate a very low affinity constant for dimerization. Nevertheless, these results suggest that the ester represents a more conservative substitution than N-methyl amino acid and more fully preserves the geometry of the unmodified peptide bond. Therefore, the inability of Aβ16-20e to form fibrils, in contrast to the ability of Aβ16-20 to do so, is attributable mainly or completely to the loss of two hydrogen bonding sites resulting from the use of ester bonds in place of amide bonds in the peptide backbone.

The similar inhibitory properties of Aβ16-20e compared to Aβ16-20m also suggest that interfering with hydrogen bonding is sufficient to prevent Aβ1-40 fibrillogenesis and that steric contributions from the N-methyl group are not required. Crosslinking experiments demonstrate that primarily one Aβ16-20-Bpa binds to each Ad 1-40 peptide. Based on the DPH fluorescence experiments and the electron microscopy, it is likely that the Aβ16-20e peptide is interacting with an oligomeric, rather than monomeric, form of Aβ1-40.

The detailed pathway of Aβ1-40 aggregation is incompletely described. Current data support a nucleation-polymerization model which proposes that below a critical concentration of Aβ1-40 the peptide is monomeric and does not aggregate (reviewed by Harper, 1997). If the critical concentration is exceeded then small nuclei form during a slow, lag phase. These nuclei then “seed” the rapid self-assembly of additional Aβ1-40 during the polymerization phase. A number of intermediates, variously termed oligomers, prefibrils and protofibrils, have been postulated to exist at points during fibrillogenesis. None of these intermediates have been isolated or characterized, however. The temporal association of these intermediates is also unclear.

Glabe and associates have shown that Aβ1-40 forms a micelle-like structure that binds DPH. Neutron and light scattering experiments have identified a micelle-like Aβ1-40 oligomer that is composed of approximately 30-50 peptides and forms early on the fibrillogenesis pathway (Yong, 2002; Lomakin, 1996; Lomakin, 1997). Temporal analysis of the fibril length distribution suggests that this micelle structure may be the center of fibril nucleation (Lomakin, 1996). It is not clear, though, if this is the same oligomer that interacts with DPH. Crosslinking Aβ1-40 with a variety of reagents typically reveals a banding pattern with a monomer-hexamer stoichiometry (Levine, 1995; Bitan, 2001). It is not clear if the scattering and crosslinking experiments are both monitoring the same intermediate. Likewise, it is not known which intermediates interact with DPH.

Aβ16-20e blocks the polymerization of Aβ1-40 before the formation of the species that binds thioflavin T. Others have reported that Aβ1-40 forms a DPH-binding, micelle-like structure with a “cmc” ≈100 ttM. Ad 16-20e functions by associating with the intermediate that binds DPH. Addition of Aβ16-20e to a molar excess of 40:1 compared to Aβ1-40 had little effect on DPH fluorescence, suggesting that the addition of Aβ16-20e was compatible with preservation of a micelle-like structure. Furthermore, at the concentrations of Aβ16-20e and Aβ1-40 used in this experiment, Aβ16-20e forms a crosslinkable, equimolar complex with Aβ1-40. Since the complex of Aβ16-20e and Aβ1-40 is stable in solution, our data suggest that the Aβ16-20e peptide stabilizes a micelle-like—i.e., DPH binding—form of Aβ1-40, in such a way that the complex does not progress toward the formation of fibrils.

The elimination of two amide protons in Aβ16-20e is sufficient to prevent this peptide from forming amyloid fibrils. Disruption of hydrogen bonding, however, cannot fully explain the efficacy of Aβ16-20m and Aβ16-20e as fibrillogenesis inhibitors. The sequence specificity of the inhibitors suggests that sidechain interactions are also critical for inhibition of Aβ1-40 fibrillogenesis. Two broad categories of mechanisms are contemplated by which both the N-methyl amino acid and ester-containing peptides inhibit fibril formation. In Mechanism A, the peptide binds to a growth site of the fibril and forms a complex with Aβ1-40. It is possible that such a complex, containing the inhibitor and Aβ1-40, could then dissociate from the fibril. Mechanism B holds that the fibril is a dynamic structure, in which fibrillar Aβ1-40 is in a slow equilibrium with a pool of soluble peptide, such that a small fraction of the Aβ1-40 can bind and dissociate from the fibril growth site. The reversible nature of Aβ1-40 fibrillogenesis, in fact, has been demonstrated experimentally in an in vitro model system of plaque growth (Maggio, 1992). According to Mechanism B, the inhibitor binds Aβ1-40 in solution and forms a stable complex, which traps Aβ1-40 in solution and prevents it from re-depositing onto the fibril.

While both mechanisms are possible, the crosslinking data indicate that Aβ16-20e is capable of binding to Aβ1-40 in a non-fibrillar state, i.e., immediately after Aβ1-40 is added to a solution of the inhibitor and before Aβ1-40 has time to form fibrils. This observation favors Mechanism B, though it remains possible that the inhibitor could also bind to fibrillar Aβ1-40, as in Mechanism A. However, Mechanism A also appears less likely a priori, since it supposes that the inhibitor peptides, with their small size and limited number of sites for interaction with Aβ1-40, are able effectively to strip Aβ1-40 from the fibril. On the contrary, one would expect the fibril to offer more interactions to a molecule of Aβ1-40 than the small peptide could. It appears likely that Aβ16-20e competes effectively with the fibril for Aβ1-40 not only through its meager complement of hydrogen bonding sites, but also through side chain interactions, perhaps in one or more solvent-exposed, hydrophobic domains of non-fibrillar Aβ1-40, e.g., the hydrophobic core domain (residues 17-21).

Incorporation of ester bonds into the Aβ16-20 peptide prevents it from aggregating and forming amyloid fibrils. By placing the ester bonds in alternating positions, Aβ16-20e was designed to display, in a β-strand conformation, one normal hydrogen bonding face and one face with diminished hydrogen bonding capabilities due to the absence of amide protons. While this modification prevented the peptide from forming amyloid fibrils, mass spectrometry and crosslinking demonstrated that Aβ16-20e is still able to form a dimeric species in solution. This feature contrasts with Aβ16-20m, in which the N-methyl groups appear to strongly disfavor self-association, even at the level of a dimer. The Aβ16-20e peptide also inhibits the fibrillogenesis of Aβ1-40 and disassembles preformed Aβ1-40 fibrils.

In addition, the inventors contemplate the synthesis of other polypeptides to inhibit fibril formation and/or to mediate the disassembly of virtually any fibril forming protein. The invention is therefore not limited to the examples described above, and as will be recognized by one of ordinary skill in the art, encompasses inhibitors and dissassemblers to all fibril proteins.

### EXAMPLES

The following examples are included to demonstrate embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many, changes can be made in the specific embodiments which are disclosed and still obtain a like or similar result without departing from the spirit and scope of the invention.

### Example 1

A. Peptide Synthesis, Purification And Analysis

The human Aβ40 peptide was synthesized using standard 9-fluorenylmethoxycarbonyl chemistry on an Applied Biosystems model 431A peptide synthesizer:

NH₂-DAEFRHDSGY¹⁰ EVHHQKLVFF²⁰ AEDVGSNKGA³⁰ IIGLMVGGVV^{β40}—COOH

A fibril forming peptide (Forlorn et al., 1993) derived from the human prion protein, amino acids 106-126 was synthesized with a free carboxyl terminus:

NH₂—¹⁰⁶KTNMK¹¹⁰HMAGAAAAGA¹²⁰ VVGGLG¹²⁶-COOH

Peptides with a carboxamide at the C-terminal were prepared by using FMOC-amide MBHA resin (Midwest Biotech). The N-methyl peptides were synthesized manually using 9-fluorenylmethoxycarbonyl chemistry and an amide MBHA resin (Midwest Biotech). Amino acids added after N-methyl amino acids (Novabiochem) were coupled for 3-5 hours using the HATU (PE Biosystems) activating reagent. Other residues were coupled for 1.5 hours with HOBt/DCC (PE Biosystems). N-methyl anthranilic acid was coupled to the N-terminal of peptides using standard chemistry and coupling times. The N-terminal of the peptides were acetylated with a 10% acetic anhydride solution in DMF. The radioactive A116-20m peptide was prepared by acetylation with ¹⁴C-acetic anhydride (Amersham).

The peptides were purified using a reverse-phase, C18 preparative HPLC column (Rainin Dynamax) at 60° C. Peptide purity was greater than 95% by analytical HPLC (Rainin C18 column). The molecular masses of the peptides were verified with electrospray mass spectrometry.

### Example 2

A. Design and Synthesis of Fibrillogenesis Inhibitor Peptides: Aβ16-22 and Variants of Aβ40 β16-22

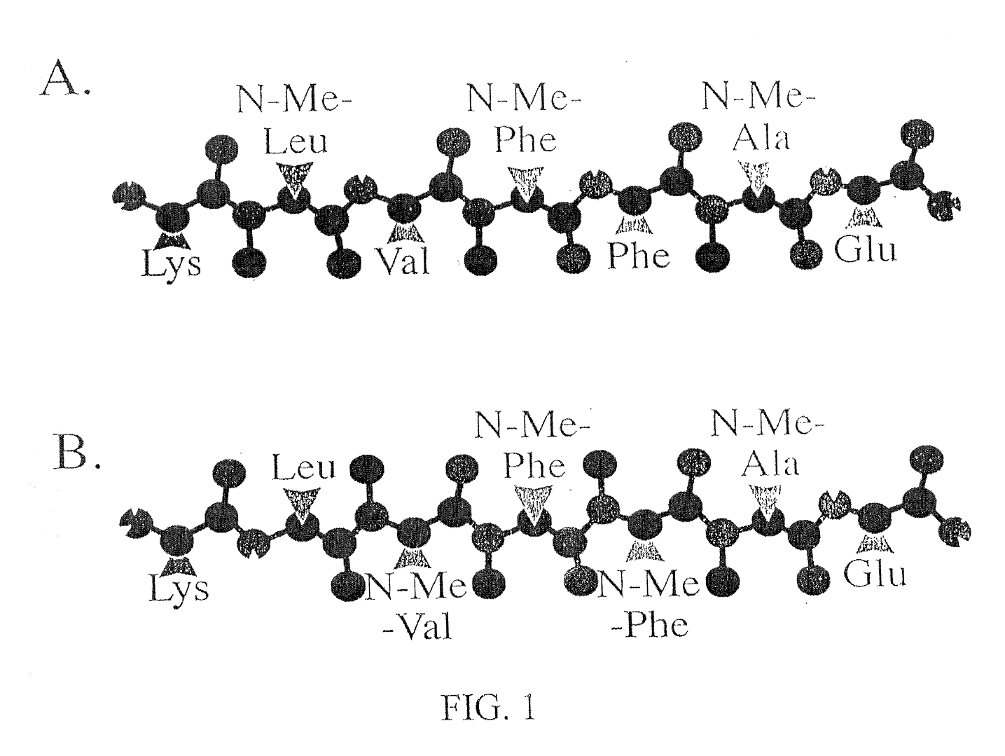

The peptides described below are based on the central, hydrophobic “core domain” of Aβ1-40 that is critical for fibril formation, since alteration of this domain abrogates fibrillogenesis (Hilbich et al., 1992; Wood et al, 1995). The strategy was to incorporate N-methyl amino acids into alternate positions of this short peptide. In a β-sheet, alternate amide protons and carbonyl oxygens are oriented to opposite sides of the peptide backbone. Thus, a peptide containing an alternation of ordinary amino acids and N-methyl amino acids, when in the β-strand (or extended) conformation, should have one “face” containing ordinary amino acids and one “face” containing N-methyl amino acids (FIG. 1A and FIG. 1B).

Table 1 lists the synthesized peptides. Peptide I (Aβ16-22) consists of amino acids β116-22 of A, and an amidated C-terminus, but contains no N-methyl amino acids. Peptides II and III (Aβ16-22m and Aβ16-22mR, respectively) contain N-methyl amino acids at alternate residues; thus these two peptides are predicted to act as inhibitors of fibrillogenesis. These two peptides differ from each other in the placement of the two charged residues, Aβ16-22m preserving and Aβ16-22mR reversing the positions of these two amino acids found in natural A. Peptides IV and V (Aβ16-22m(4) and PrPm, respectively) also contain N-methyl amino acids, but are predicted not to act as inhibitors of AR fibrillogenesis. Aβ16-22m(4) has the same sequence as the previous three peptides, except that it contains N-methyl amino acids at consecutive rather than alternate positions. Consequently, if this peptide formed a R-strand, it would have N-methyl amino acids on both faces of the peptide backbone and would be predicted not to interact with Aβ40. PrPm has N-methyl amino acids at alternate positions, but the sequence is from an unrelated protein (albeit another fibril forming one), the human prion protein. In all cases, the peptides were synthesized with amidated C-termini.

Yields from syntheses of peptides containing N-methyl amino acids are not adequate if coupling reagents from standard FMOC chemistry are used (Coste et al., 1990; Coste et al., 1991). For this reason, the activating reagent HATU was required for the coupling steps immediately after an N-methyl amino acid (Coste et al., 1990; Coste et al., 1991; Carpino et al., 1994; Carpino, 1993). The use of this reagent gave excellent purity and yields of the target peptides.

The N-methyl amino acid containing peptides are surprisingly soluble, and solutions could be prepared with peptide concentrations exceeding 140 mg/ml at physiological pH (7.4) and salt concentration (150 mM). In contrast, the corresponding unmethylated peptides are soluble at concentrations up to Al-2 mg/ml, i.e., twenty to forty-fold less soluble under similar conditions. In view of the increased hydrophobicity and the diminished hydrogen bonding potential of the N-methylated peptide, its excellent solubility in water was unexpected.

TABLE 1
Summary of Peptides Synthesized

| Peptide | | Sequence |
|---|---|---|
| I | Aβ16-22 | NH₂-KLVFFAE-CONH₂ |
| II | Aβ6-22m | NH₂-K(me-L)V(me-F)F(me-A)-E-CONH₂ |
| III | Aβ16-22mR | NH₂-E(me-L)V(me-F)F(me-A)-K-CONH₂ |
| IV | Aβ16-22m(4) | NH₂-KL(me-V)(me-F)(me-F)(me-A)-E-CONH₂ |
| V | PrPm | NH₂-GA(me-A)AAA(me-V)V-CONH₂ |
| VI | Ac-Aβ16-22 | Ac-NH-KLVFF-CONH₂ |

The inhibitor peptides Aβ16-22m and Aβ16-22mR were designed to present two faces when in the R-strand (extended) conformation: a “binding face” and a “blocking face”. The periodicity of a β-strand makes it an inherently repetitive structure. Amphiphilic β-strand peptides, for example, have alternating hydrophilic and lipophilic amino acids (Osterman et al., 1984). This repetitive nature of β-strands allows for the design of peptide with faces of different characters, by the strategic placement of modifications. In the peptides described in this invention, N-methyl amino acids were used to form the “blocking face” because the methyl group removes a backbone hydrogen bond interaction between β-strands in a β-sheet. In addition, the N-methyl amino acids are sterically hindered and tend to be restricted in their backbone conformations to the β-sheet geometry (Manavalan and Momany, 1980; Tonelli, 1970; Tonelli, 1971; Tonelli, 1974; Vitoux et al., 1986; Kumar et al., 1975; Patel and Tonelli, 1976). The need for the N-methyl amino acids to alternate was shown by the fact that Aβ16-22m(4), a homologous peptide containing four consecutive N-methyl amino acid residues, was only a weak inhibitor. Furthermore, the fact that PrPm also was not an inhibitor suggests that alternate spacing of N-methyl amino acids was not sufficient to form an inhibitor, i.e., there is also a need for the inhibitor to have sequence homology to the fibril forming peptide.

### Example 3

A. Fibrillogenesis and Fibril Disassembly Assays Aβ16-22 Variants

Fibril inhibition and disassembly activities of inhibitor peptides was measured using standard techniques as described herein.

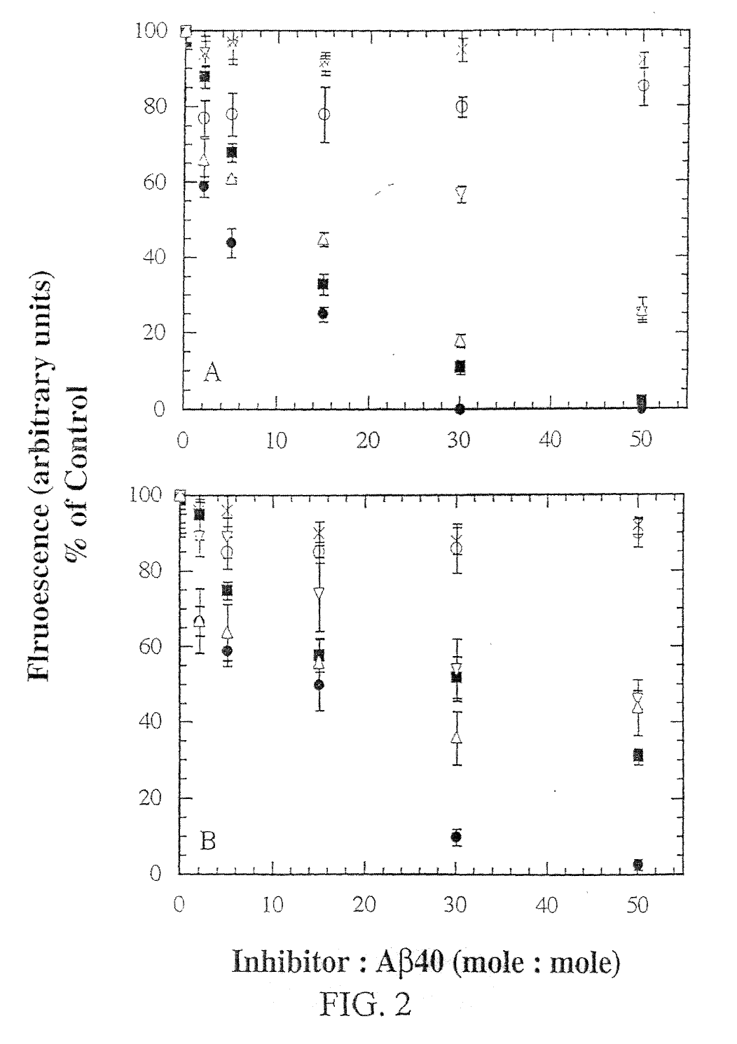

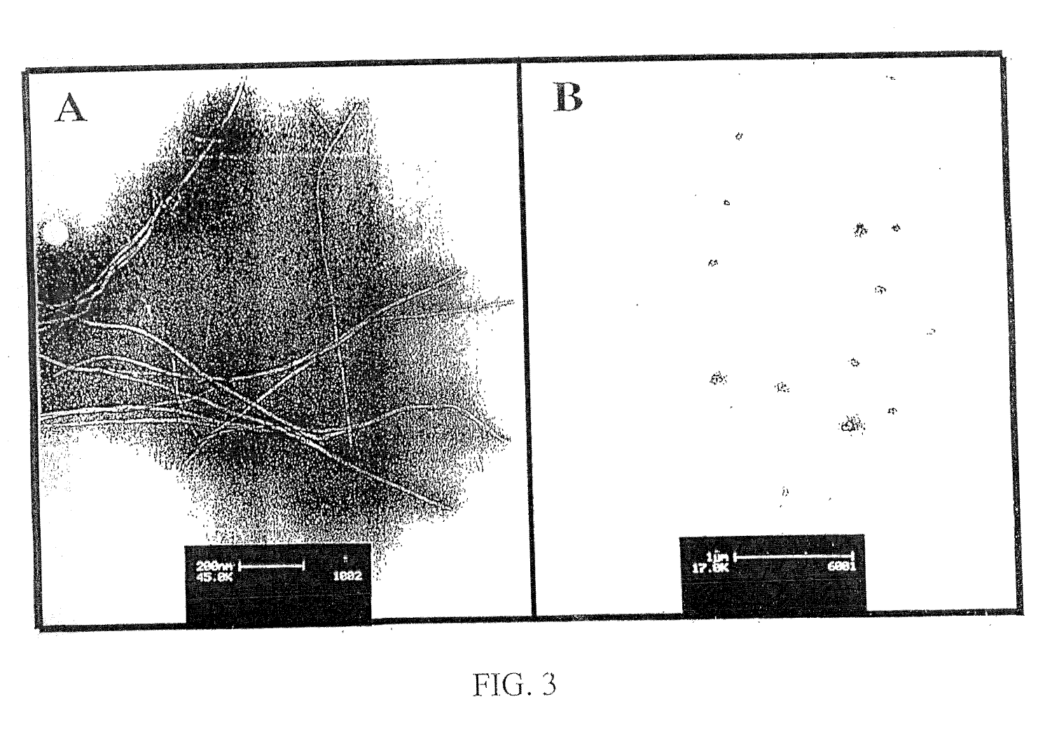

Two of the N-methyl peptides, Aβ16-22m and Aβ16-22mR, prevented fibril formation of Aβ40 in a dose dependent manner, in vitro. These are the two peptides containing N-methyl amino acids in alternating positions of the sequence. FIG. 2A shows thioflavin fluorescence as a function of inhibitor concentration; since a constant concentration of Aβ40 peptide was used, this was expressed as the molar ratio of inhibitor:Aβ40 peptide.

In order to compare relative potency of the peptides, data for both inhibition of fibrillogenesis and disassembly of pre-formed fibrils were fit to a simple equation. Values of the two parameters for each of the peptides are listed in Table 2. Both Aβ16-22m and Aβ16-22mR were effective inhibitors of fibrillogenesis; the IC50 of Aβ16-22m and A6-22mR occurred at inhibitor:Aβ40 ratios of 4:1 and 9:1, respectively. Incubation with greater than a 30-fold molar excess of Aβ16-22m resulted in complete elimination of thioflavin fluorescence; for Aβ16-22mR, this occurred at higher ratios, 50:1. The Aβ16-22m(4) peptide, containing four N-methyl amino acids, but in consecutive rather than alternating positions, weak inhibitor of A fibrillogenesis, having an IC₅₀ ratio in excess of β40:1. The unmethylated control peptide, Aβ16-22, had a relatively modest inhibitory effect on fibril formation. As shown in FIG. 2A, at concentrations at which Aβ16-22m inhibited fibrillogenesis completely, the unmethylated Aβ16-22 inhibited fibrillogenesis by approximately 10-20%. Finally, an unrelated, methylated peptide, PrPm, had no effect on Aβ40 fibril formation.

TABLE 2
Summary of Fibrillogenesis Inhibition
and Fibril Disassembly Data

| | |
|---|---|
| Inhibition of | Fibril |
| Fibrillogenesis | Disassembly |

| Peptide | IC₅₀ | ICₘₐₓ | IC₅₀ | ICₘₐₓ |
|---|---|---|---|---|
| Aβ6-22m | 4.2 | 100 | 6.9 | 100 |
| Aβ16-22mR | 7.8 | 100 | 23.7 | 100 |
| Aβ16-22m(4) | 38.9 | 100 | 31.6 | 100 |
| PrPm | 6.0 | 8.6 | 8.9 | 10.3 |
| Ac-Aβ16-20 | 8.4 | 100 | 11.3 | 100 |
| Aβ16-22 | 1.1 | 23.0 | 11.3 | 89.2 |

These results were confirmed by electron microscopy, which demonstrated a complete lack of fibrils in Aβ40 samples with a 30-fold molar excess of inhibitor (FIG. 3A and FIG. 3B); EM showed round particles which may be complexes of Aβ40 and Aββ16-22m. Inhibition of fibril formation was also confirmed with a Congo Red-binding solution assay.

The inhibitor peptides, Aβ16-22m and A6-22mR both were also able to disassemble pre-formed Aβ40 fibrils. After incubation of Aβ40 for seven days to form fibrils, different concentrations of the inhibitor peptides were added to the fibril solution. The extent of disassembly was then quantitated using the thioflavin assay after three additional days of incubation at 37° C. The IC₅₀ for the disassembly occurred at inhibitor:Aβ40 ratios of approximately 10:1 and 25:1 for Aβ16-22m and Aβ16-22mR, respectively (FIG. 2B). As was observed for inhibition of fibril formation, the remaining peptides either disaggregated fibrils weakly or did not do so.

In order to facilitate comparison of the data with those obtained for other fibrillogenesis inhibitors using different variations of methodology, the inventors synthesized and tested, using the techniques described herein, a known fibrillogenesis inhibitor, that of Tjernberg et al., 1996, listed as Peptide VI (Ac-Aβ16-22) in Table 2. As with the other peptides reported above, the inventors examined a range of inhibitor concentrations, using a standardized concentration of A1340 known to lead to fibril formation with predictable yields and kinetics, and expressed the results in terms of an inhibitor:A molar ratio. As shown in FIG. 2A and FIG. 2B, Ac-Aβ16-22 did indeed inhibit Aβ40 fibrillogenesis, and disassembled pre-formed Aβ.(40) fibrils. The IC₅₀ occurred at an inhibitor:A ratio of 10:1, in basic agreement with the results of Tjernberg et al. By the criterion of the IC₅₀, Ac-Aβ16-22 was highly effective for inhibiting fibrillogenesis and disassembling pre-formed fibrils, though slightly less so than Aβ16-22m or Aβ16-22mR.

The Aβ16-22m and Aβ16-22mR peptides fulfill the design requirements for a fibrillogenesis inhibitor. In addition to inhibiting fibrillogenesis, these peptides also caused disassembly of pre-formed A4β40 fibrils. The latter feature is in common with some well studied inhibitors of fibrillogenesis or crystallization (e.g., polymerization of hemoglobin S (Osterman et al., 1984), and calcium oxalate crystallization (Eaton and Hofrichter, 1990), among others), and suggests reversibility of many of the steps of Aβ fibrillogenesis.

### Example 4

A. Analytical Ultracentrifugation OF Aβ16-22 and Aβ16-22 Variants

A number of small peptides derived from the full length A are capable of aggregating and forming fibrils.

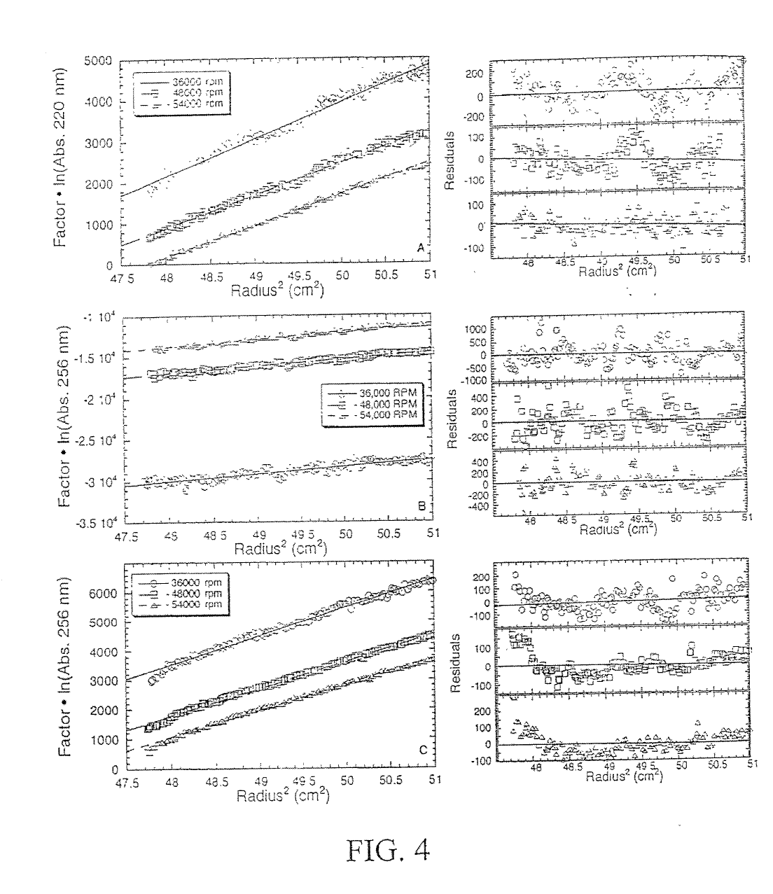

Analytical ultracentrifugation, consequently, was used to determine if Aβ136-22m aggregates, either as an oligomer or fibrillar species. Studies were conducted at three different peptide concentrations and at three different rotor speeds (FIG. 4). Modeling the data as a single ideal species resulted in the best agreement with the theoretical curves. Table 3 summarizes the molecular values obtained from the analysis of the different data sets. The average molecular weight is 870 f 10, which is similar to the calculated weight of 893.9.

TABLE 3
Summary of Analytical Ultraceutrifugation Data

| | 36,000 RPM | 48,000 RPM | 54,000 RPM |
|---|---|---|---|
| 100 μm Aβ16-22m | 904 ± 9 | 796 ± 5 | 767 ± 4 |
| 500 μm Aβ16-22m | 858 ± 29 | 825 ± 15 | 904 ± 14 |
| 5 mM Aβ16-22m | 969 ± 7 | 919 ± 4 | 888 ± 5 |

### Example 5

A. Circular Dichroism Aβ16-22 and Aβ16-22 Variants

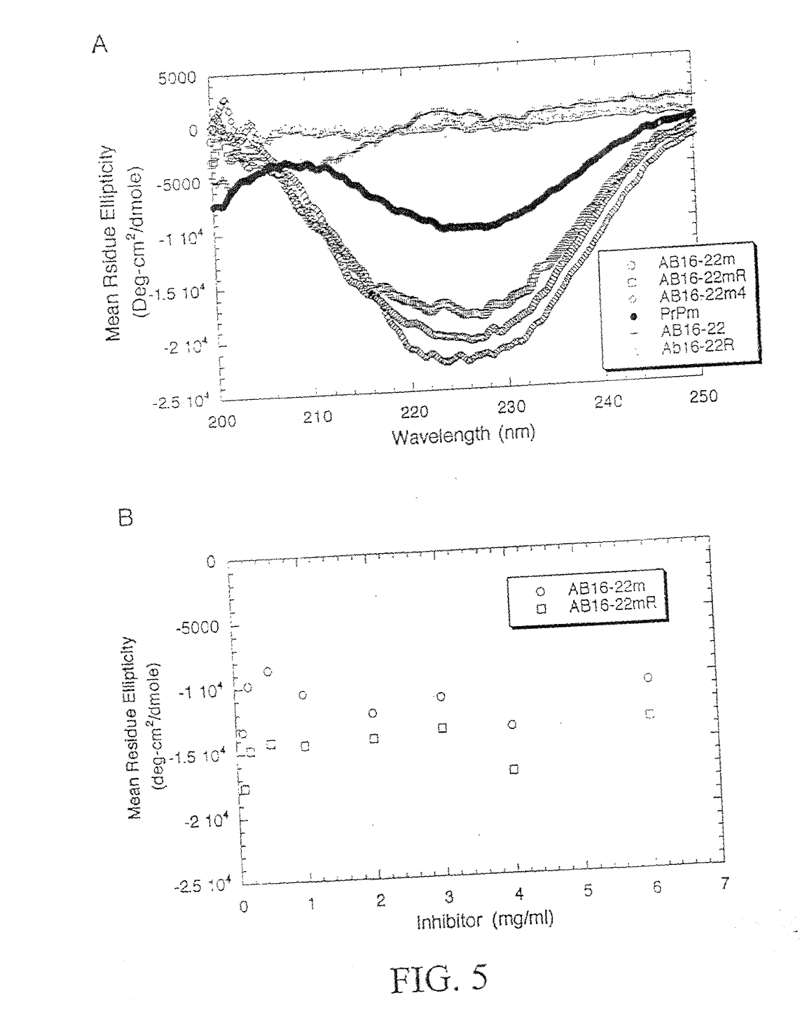

Peptides containing N-methyl residues are restricted in their backbone conformations; N-methyl amino acids destabilize-helices and tend to promote the ββ-sheet (Manavalan and Momany, 1980; Patel and Tonelli, 1976). The CD spectra of Aβ316-22m and Aβ16-22mR, both of which have three N-methyl amino acids, is characteristic of a β-sheet secondary structure except that the minimum is shifted to 226 nm (FIG. 5A). Similar red-shifted (3β-sheet spectra have been observed for a number of other peptides, and this shift has been attributed to the twist of the β-sheet sheet (Orpiszewski and Benson, 1999; Cerpa et al., 1996; Manning et al., 1988; Zhang and Rich, 1997). In the case of the inhibitor peptide, however, it was also possible that the methyl groups were affecting the electronic properties of the peptide bond, and hence, their transitions observed by CD spectroscopy. Red-shifted minima have also been observed for other peptides containing N-methyl amino acids (Chitnumsub et al., 1999; Nesloney and Kelly, 1996). In contrast to the N-methyl peptides, the CD spectrum of the unmethylated, control peptide AR β316-22 was that of a random coil in solution. The mean residue ellipticity of Aβ16-22m at 226 nm, the minimum observed in the CD spectra, was independent of concentration (FIG. 5B) between peptide concentrations of 0.1 mg/ml and 6 mg/ml. This was consistent with the analytical ultracentrifugation results that demonstrated the peptide was monomeric in solution.

Both of the peptides with alternating N-methyl residues, Aβ16-22m and Aβ16-22mR, were inhibitors of fibrillogenesis, but the former peptide was consistently observed to be the more effective inhibitor. The same rank order was observed for disassembly of pre-formed ARβ40 fibrils. While these data can be accommodated by the assumption of either a parallel or antiparallel orientation of either inhibitor with respect to the Aβ40 peptide, the antiparallel orientation appears somewhat more likely for the more potent of these two inhibitory peptides, Aβ16-22m. In the case of Aβ16-22m, an antiparallel orientation would minimize unfavorable charge interactions between the Lys and Glu side chains of Aβ16-22m and Aβ40. For the less potent inhibitor, Aβ16-22mR, two possibilities would then seem to exist: 1) It too might align with the Aβ40 peptide in an antiparallel orientation, but this would result in an unfavorable charge interactions between side chains; such unfavorable charge interactions could account for its lesser potency as an inhibitor peptide. 2) Alternatively, to avoid such unfavorable charge interactions, this peptide could be aligned parallel to the Aβ40 peptide. However, if this latter possibility were true, the decreased potency of Aβ16-22mR would then suggest that in the absence of unfavorable side chain interactions, an antiparallel orientation between A and inhibitors is inherently more stable than the parallel orientation.

### Example 6

A. Chymotrypsin Digestion of A6-22 and Aβ16-22 Variants

The peptides were dissolved in 0.5% ammonium bicarbonate at a concentration of 1.0 mg/ml. The pH of the solution was 8.4. Chymotrypsin (Worthington Biochemical Corporation) was added to the peptide solutions so that the final concentration was 0.1 mg/ml. Samples were incubated at 37° C. After twenty-four hrs, the samples were frozen and lyophilized. The samples were analyzed by reverse-phase HPLC using an analytical C 18 column (Rainin Microsorb) and eluted, using a 60 min gradient from 10-70% acetonitrile, containing 0.1% (v/v) TFA. The loss of intact peptide and appearance of fragments were quantitated by integration of the appropriate peaks. Results were expressed as a percent digestion of the peptides. In addition, identities of the peaks were confirmed by electrospray mass spectrometry.

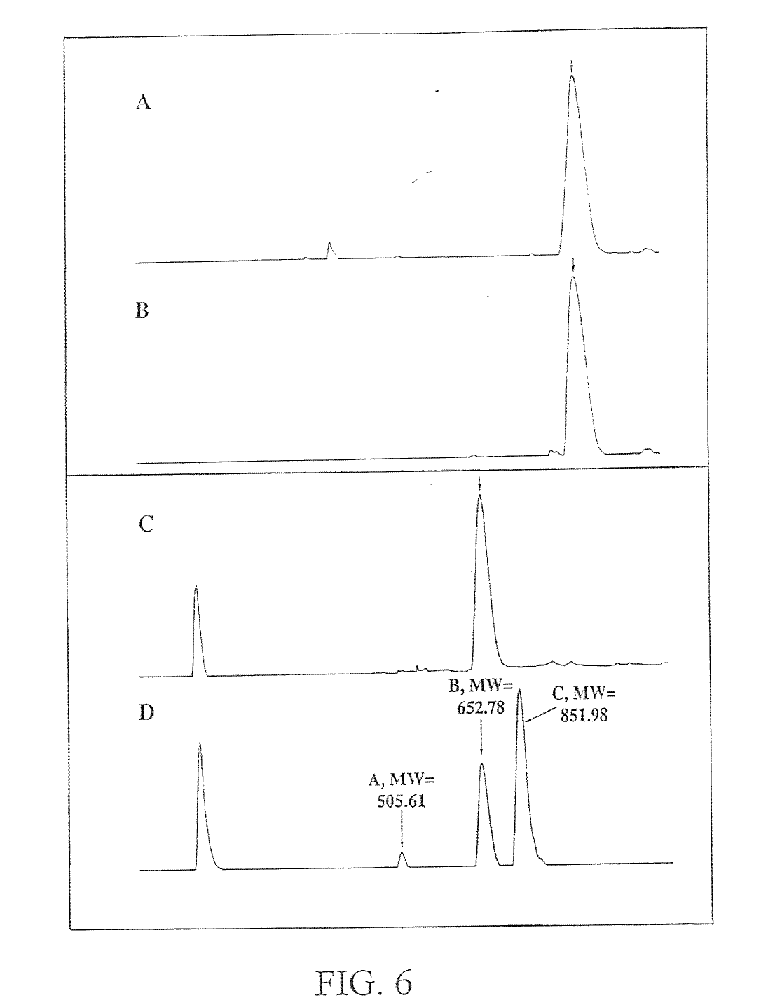

Small peptides are often highly sensitive to proteolytic degradation, and this was indeed the case for Aβ16-22. This unmethylated peptide contained a predicted chymotryptic cleavage site, and was shown to be cleaved by chymotrypsin (FIG. 6C and FIG. 6D). The molecular mass of peptides are shown in the peaks, as determined by mass spectrometry. Peak A, eluting at 16.8 mins, had a molecular mass of 505.61, consistent with the predicted molecular mass of 506.4 for NH₂—KLVF-OOOH; Peak B, eluting at 22 mins had a molecular mass of 851.98, consistent with the predicted molecular mass of 852.6 for the intact starting peptide, NH₂—KLVFFAE-CONH₂; and Peak C, eluting at 25 mins, had a molecular mass of 652.78, consistent with the predicted molecular mass of 653.5 for NH₂—KLVFF—COOH. In contrast, Aβ16-22m exhibited complete resistance to chymotrypsin digestion over a period of 24 hrs (FIG. 6A and FIG. 6B).

Aβ16-22m and Aβ16-22mR also possess two other traits of potential relevance to the development of a therapeutic agent. First, they are highly soluble in aqueous solutions. This may be surprising in view of the added hydrophobicity attributable to the N-methyl group, and due to the removal of one potential site of hydrogen bonding between the peptide and water. Nevertheless, the N-methyl peptides are 20-040 times more soluble than the unmethylated congeners. Indeed, this appears to be a trait in common for all N-methyl peptides the inventors have studied, as both Aβ16-22m(4) and PrPm were also highly soluble in water. Second, Aβ16-22m is highly resistant to proteolytic digestion. Although the unmethylated congener, Aβ16-22, contains a scissile peptide bond, the methylated peptide was completely resistant to chymotrypsic digestion. Protease resistance has been observed for other N-methyl amino acid-containing peptides (Haviv et al., 1993; Dragovich et al., 1999) and may be a general trait.

### Example 7

A. Design and Synthesis of Aβ16-20 and Aβ16-20 Variants

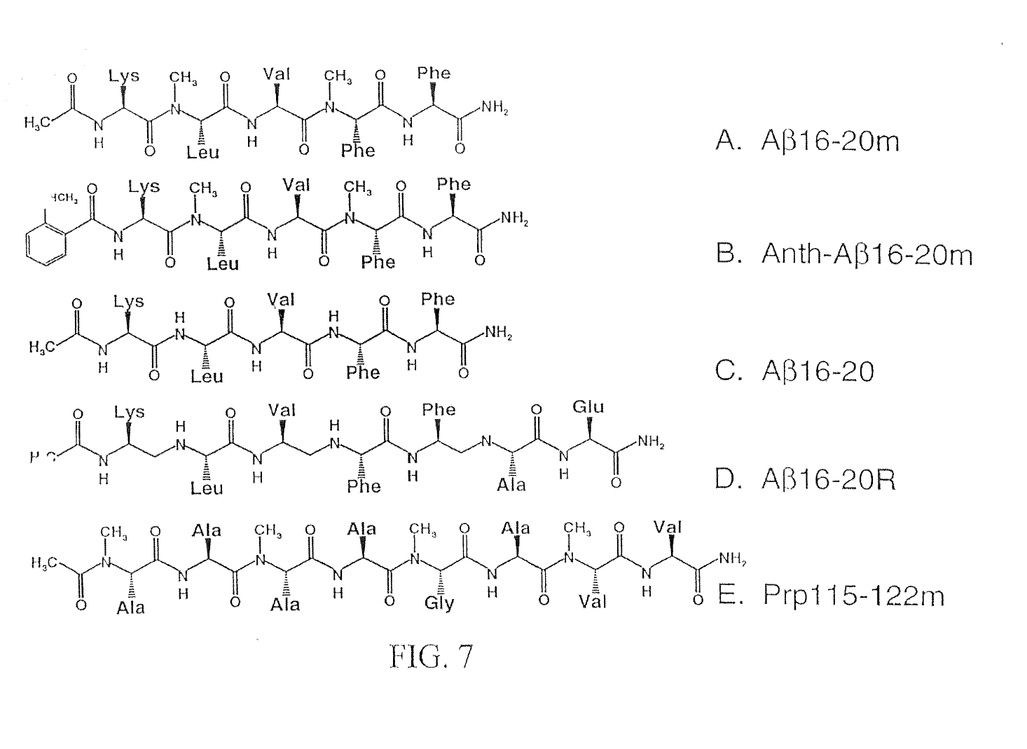

The structures of the peptides are illustrated in FIG. 7A, FIG. 7B, FIG. 7C, FIG. 7D and FIG. 7E. N-methyl amino acid-containing peptides were synthesized using HATU activation for residues after N-methyl amino acids 32-35. N-methyl anthranilic acid was treated as a normal amino acid and coupled using HOBt/DCC chemistry without protection of the secondary amine.

The Aβ16-20m peptide (FIG. 7A) resembled the previously described inhibitor of Aβ40 fibrillogenesis, Aβ16-22m. Both Aβ16-20m and A(16-22)ₘ were homologous to the central region of A (residues β16-22) and contain alternating methyl groups, which were designed to inhibit A fibrillogenesis and disassemble pre-formed fibrils 20. That is, these peptides were designed so that, as β-strands, they present one “face” that formed hydrogen bonds with A peptides, but a second “face” in which the ability to form hydrogen bonds was severely reduced through the replacement of amide hydrogens by methyl groups. Aβ16-20m was also designed as a potentially membrane permeable analogue of Aβ16-22m, since Aβ16-20m was more hydrophobic and had a net charge of +1 at neutral pH, as opposed to the net charge of zero for Aβ16-22m. Furthermore, Aβ16-20m was labeled with the fluorescent probe N-methyl anthranilic acid (Jureus et al., 1998) to create the Anth-Aβ16-20m peptide (FIG. 7B). The Aβ16-20 peptide (FIG. 7C) was synthesized as a positive control because another group has demonstrated that it is an effective inhibitor of beta amyloid fibrillogenesis (Tjernberg et al., 1997).

Reduced peptides were synthesized to demonstrate that the methyl groups in these peptides conferred conformational rigidity on the peptide backbone. The synthesized, reduced peptide, or pseudopeptide homologue of A(β16-22)ₘ, was called Aβ116-22R (FIG. 7D). Like A(β16-22)ₘ and A(β16-20)ₘ, when A (16-22)R was arrayed as a (3-strand, it had one face capable of forming hydrogen bonds, and one face in which some of the potential hydrogen bonding sites were altered by reduction. A(16-22)R lacked three carbonyl oxygens found in the unmodified peptide, i.e., in contrast to Aβ16-22)ₘ, which lacked three amide protons. Both of these modifications reduced potential hydrogen bonding sites by the same number. Reduced peptide bond-containing peptides were used to assess the role of conformational stability because the C—N bonds of A(β16-22)R lacked the partially double bonded character of the peptide bond. The peptide was synthesized essentially according to the procedure of Meyer et al.; (1995). The CH2-NH₂ isosteres were formed by reductive alkylation of the preformed amino aldehyde, in the presence of NaCNBH3 in 0.5% acetic acid (v/v) in DMF. Completion of the reduction was monitored by ninhydrin procedure, and took less than 3 h. Peptide synthesis, cleavage from resin and deprotection were carried out using normal FMOC chemistry procedures. Peptide was purified by preparative RP-HPLC to a purity >98%, and identity was assessed by ES-MS.

Finally, to test the sequence specificity of the N-methylated inhibitor peptides, a fibril forming peptide derived from the human prion protein (amino acids 106-126) was synthesized. As a potential inhibitor of fibril formation by this peptide, PrP115-122m (FIG. 7E) was synthesized. It had three N-methyl amino acids at alternate positions.

### Example 8

A. Fibrillogenesis and Fibril Disassembly Assays Aβ16-20 and A 16-20 Variants

A standard thioflavin assay was used to assess the fibril inhibition and disassembly activities of the inhibitor peptides.

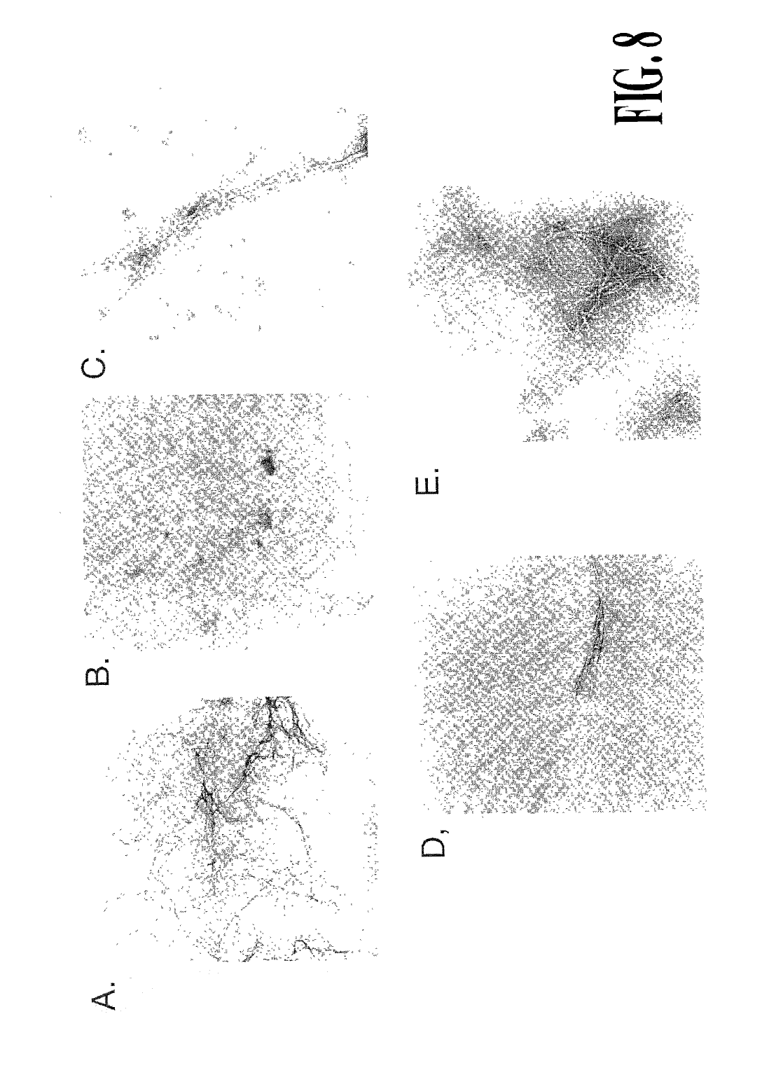

In an inhibition experiment, the inhibitor peptides were incubated at various concentrations with the Aβ40 peptide for five days at 37° C. At this point, samples without inhibitor peptide demonstrated long, unbranched fibrils by electron microscopy (FIG. 8A). Electron microscopy of samples containing Aβ16-20m did not demonstrate any fibrillar material, although some amorphous precipitate was observed (FIG. 8B). Some fibrillar material was observed in samples containing the Aβ16-20 peptide (FIG. 8C). The AR β16-20 peptide, however, formed fibrils on its own and it was not clear if the fibrils observed by electron microscopy were composed of Aβ16-20, Aβ40 or a mixture of both peptides (Tjernberg et al., 1997).

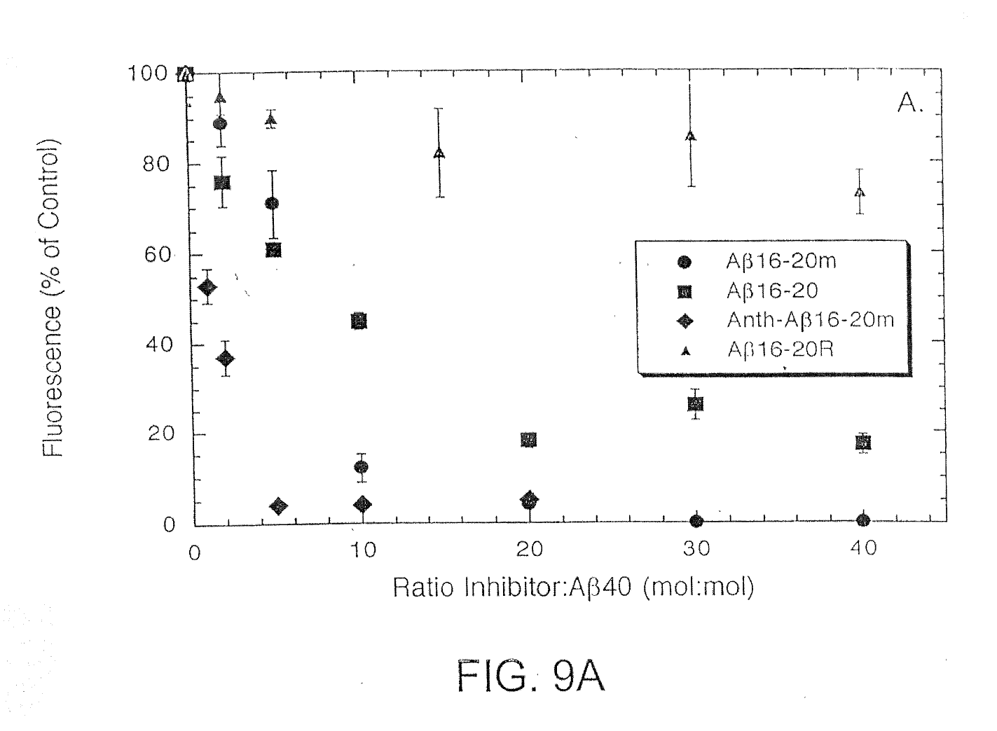

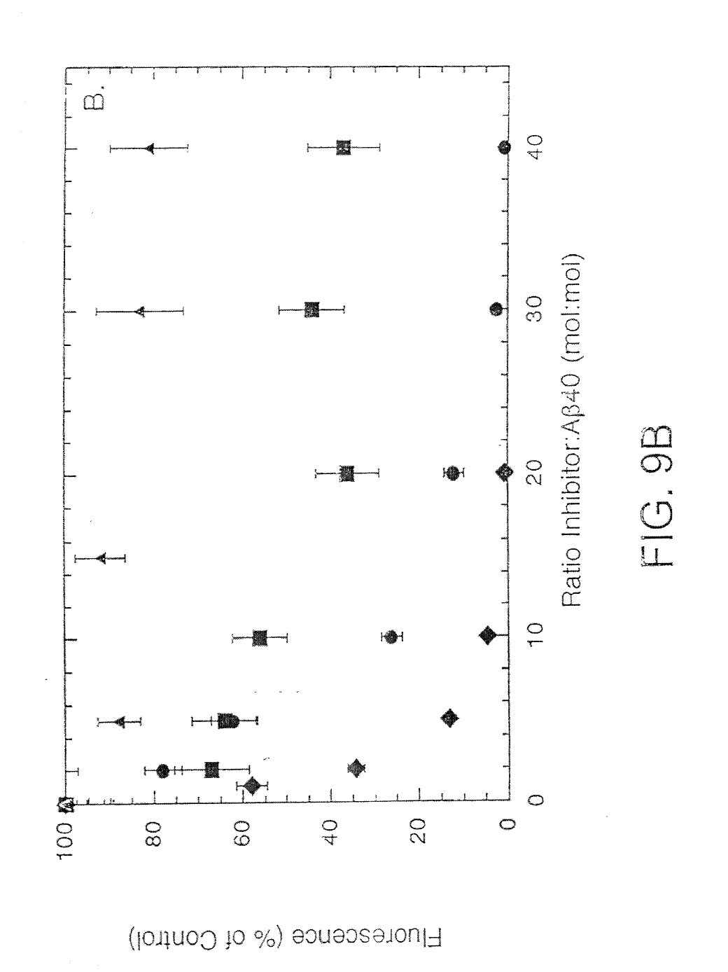

Thioflavin T fluorescence was used as a more quantitative measure of fibrillogenesis. FIG. 9A demonstrates that all three peptides inhibit the fibrillogenesis of Aβ40 in a concentration dependent manner. Since a constant concentration of Aβ40 was used, the thioflavin fluorescence was displayed as a function of the molar ratio of inhibitor peptide to Aβ40. This ratio referred to the total molar amount of each peptide and did not refer to the stoichiometry of the Aβ40 and inhibitor complex. The methylated peptides were more effective at inhibiting fibrillogenesis than the non-methylated peptide. None of these inhibitor peptides demonstrated any thioflavin fluorescence in the absence of Aβ40 peptide.

In order to facilitate comparison among the peptides, the inhibition curves were fit to the equation for a hyperbola, as is used to describe Michaelis-Menten kinetics and ligand-receptor interactions. Fitting the data to this equation yields ICₘₐₓ and IC₅₀, parameters analogous to the Vₘₐₓ and Kₘ, respectively, of enzyme kinetics; however, the use of this equation did not favor a specific model for inhibition. The IC₅₀ and ICₘₐₓ values for the different inhibitors are summarized in Table 4. Aβ16-20, Aβ16-20m and Anth-Aβ16-20m exhibited IC₅₀ values at inhibitor to Aβ40 molar ratios of 5.3, 6.5 and 1.2, respectively. The ICₘₐₓ values ranged from 89-100% inhibition. These data demonstrated that all three peptides were effective inhibitors of Aβ40.

TABLE 4
Summary of Fibrillogenesis Inhibition and Fibril Disassembly Data

| | |
|---|---|
| Inhibition | Disassembly |

| Peptide | IC₅₀ | ICₘₐₓ | ICₘₐₓ | IC₅₀ |
|---|---|---|---|---|
| Aβ16-20 | 5.3 | 89 | 2.9 | 64 |
| Aβ16-20m | 6.5 | 100 | 6.1 | 100 |
| Anth-Aβ16-20m | 1.2 | 100 | 1.4 | 100 |

The effectiveness of the peptides in disassembling pre-formed Aβ40 fibrils was also examined with electron microscopy and thioflavin assays. In these experiments, Aβ40 was incubated alone for five days and then the inhibitor peptide was added and the mixture was incubated for an additional three days. The control sample without any inhibitor peptide did not exhibit any change in fibril morphology between five and eight days. Electron microscopy of samples containing Aβ16-20m did not reveal any fibrillar material after three days of disassembly and appeared identical to the inhibition samples (FIG. 8B). The Aβ16-20 peptide sample, however, did contain, significant amounts of fibrillar material (FIG. 8D), though, it was not known whether the fibrils were composed of Aβ40, Aβ16-20, or both peptides.

The fibril disassembly was also quantitated with thioflavin T fluorescence. FIG. 9B demonstrates that all of the inhibitor peptides were able to at least partially disassemble Aβ40 fibrils. These data were plotted as described for FIG. 9A. The methylated peptides were more effective at disassembling the amyloid fibrils than the non-methylated peptide. This difference between the methylated and non-methylated peptides was also observed for the inhibition of fibril assembly. The IC₅₀ values for Aβ16-20, Aβ16-20m and Anth-Aβ16-20m occurred at inhibitor to Aβ40 molar ratios of 2.9, 6.1 and 1.4, respectively (Table 6). The ICₘₐₓ ranged from 64-100%. The lowest ICₘₐₓ value, 64%, corresponds to the non-methylated peptide.

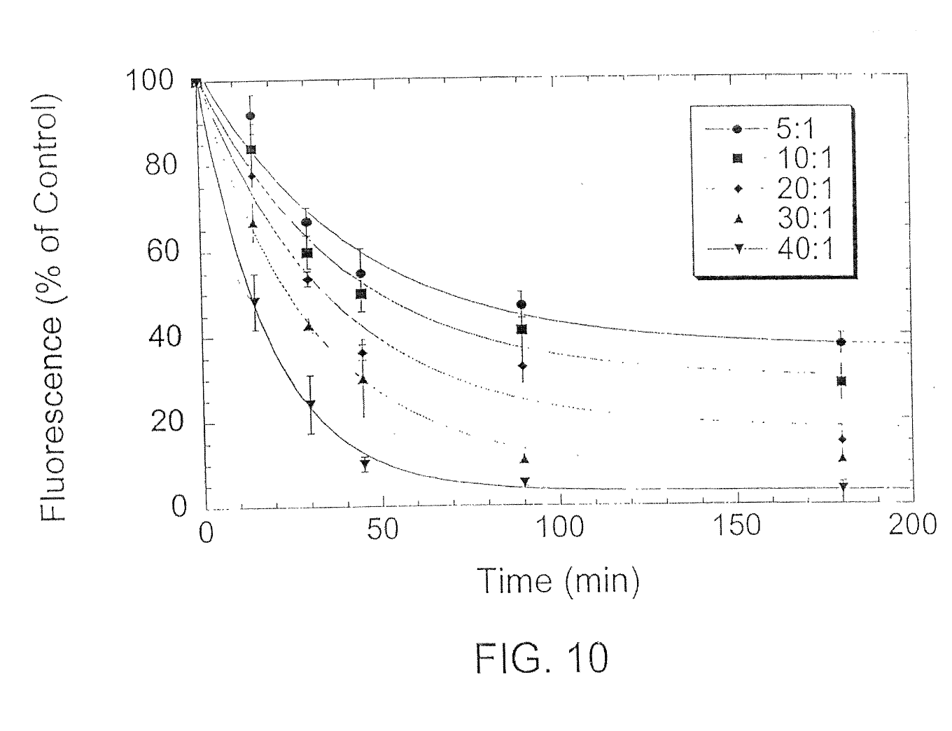

The kinetics of fibril disassembly, were also investigated using the thioflavin fluorescence assay. FIG. 10 demonstrates that Aβ16-20m disassembles pre-formed Aβ40 fibrils over a period of approximately one hour. The kinetics of fibril disassembly at all inhibitor concentrations were best fit by a first order rate law. Although the extent of disassembly depended on the concentration of inhibitor, the pseudo first-order rate constants for disassembly showed only a slight concentration dependency, most visible at inhibitor:Aβ40 ratios above 30:1 (Table 5).

TABLE 5
Summary of Aβ40 Fibril Disassembly Rates

| Molar Ratio Aβ16-20m:Apβ40 | Rate Constant (min-1) |
|---|---|
| 5:1 | 0.023 (± 0.007) |
| 10:1 | 0.025 (± 0.005) |
| 20:1 | 0.027 (± 0.006) |
| 30:1 | 0.032 (± 0.003) |
| 40:1 | 0.052 (± 0.003) |

### Example 9

A. Inhibition of Fibrillogenesis and Fibril Disassembly by N-Methyl Amino Acid-Containing Peptides is Sequence Specific

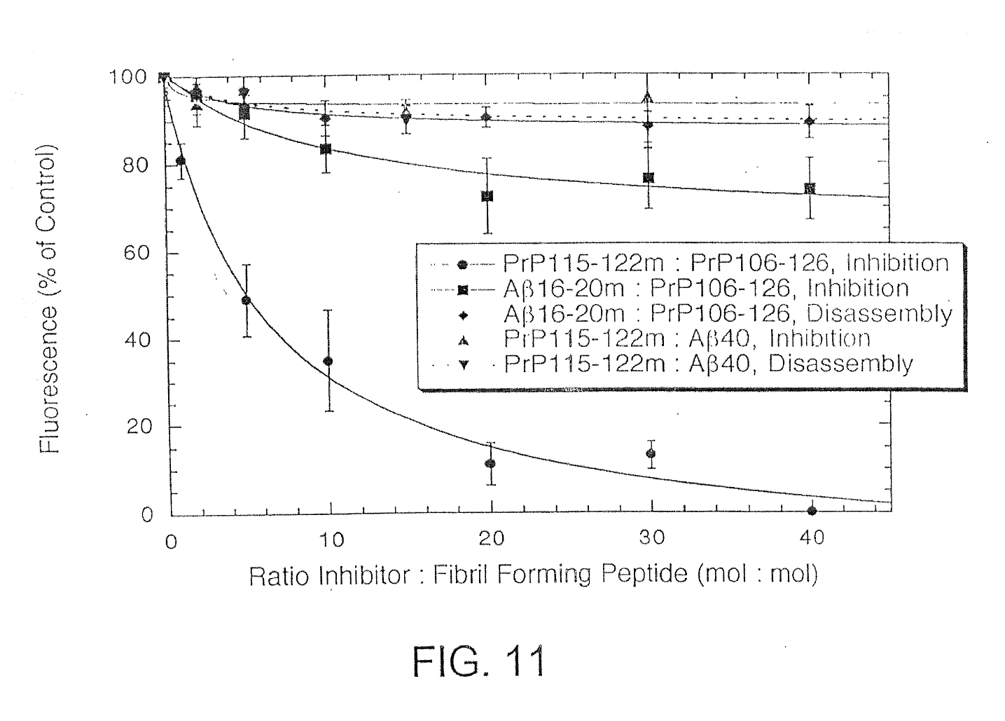

FIG. 11 illustrates the amino acid sequence specificity of the N-methyl amino acid-containing inhibitors in both fibrillogenesis and fibril disassembly.

A peptide was synthesized consisting of amino acids 106-126 of the human prion protein (Prp106-126). This peptide was previously reported to form fibrils associated with thioflavin fluorescence (Forloni, et al., 1993). The inventors also synthesized the peptide PrP 115,-122m shown in FIG. 7E, designed to inhibit fibril formation by PrP 106-126. Like Aβ16-20m and Aβ116-22m, PrP115-122m contained N-methyl amino acids in alternate residues, and had an amino acid sequence derived from the central region of the peptide of which it was designed to inhibit fibril formation.

As shown in FIG. 11, PrP115-122m was a highly effective inhibitor of fibril formation by PrP106-126, but was ineffective at inhibiting fibril formation by Aβ40. Similar results were obtained for fibril disassembly. By the same token, A(β16-20)ₘ, was ineffective as an inhibitor of PrP 106-126 fibrillogenesis but was a highly effective inhibitor of Aβ40 fibrillogenesis. These data were consistent with the notion that inhibition of fibrillogenesis and fibril disassembly by N-methyl amino acid-containing peptides is amino acid sequence specific.

### Example 10

A. The N-Methyl Amino-Containing Peptides are All Monomeric

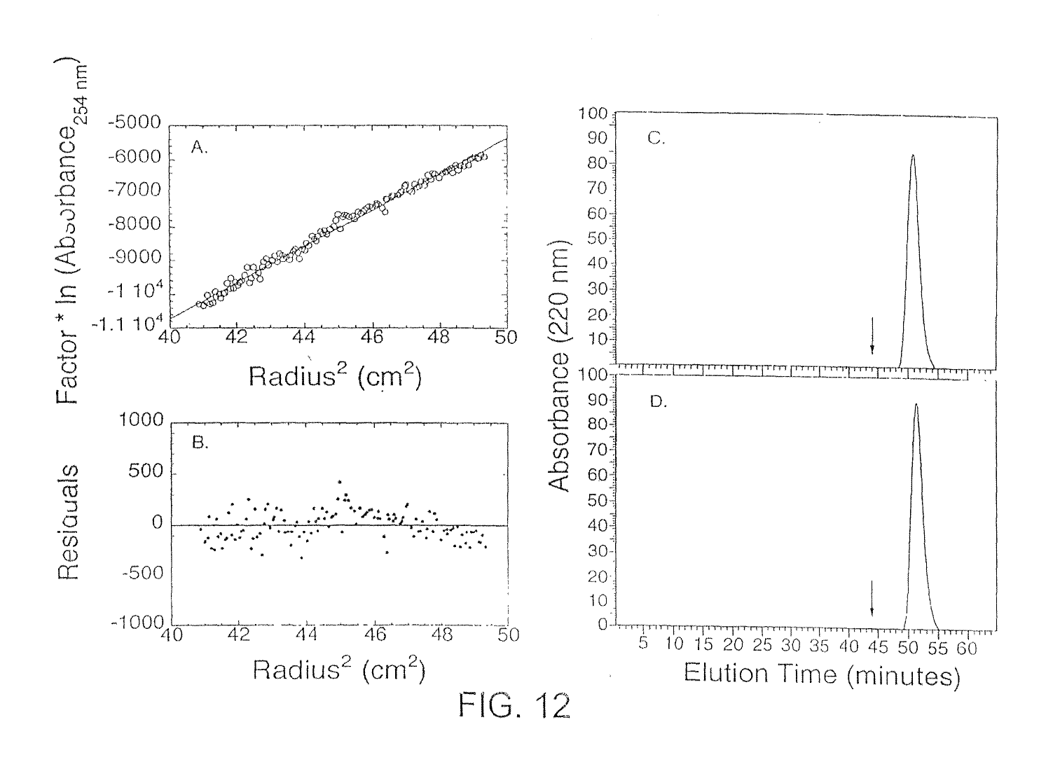

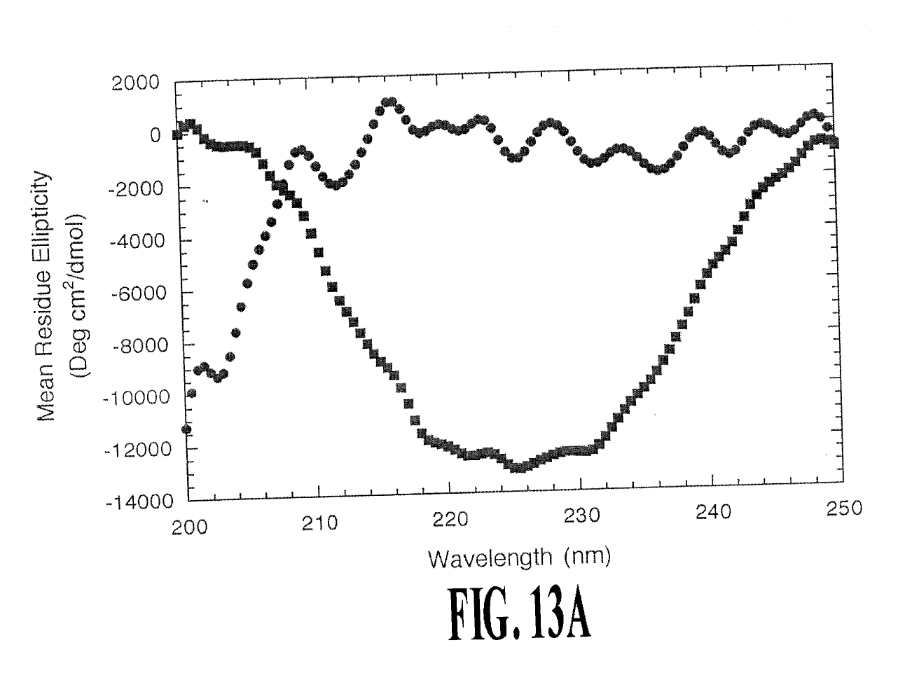

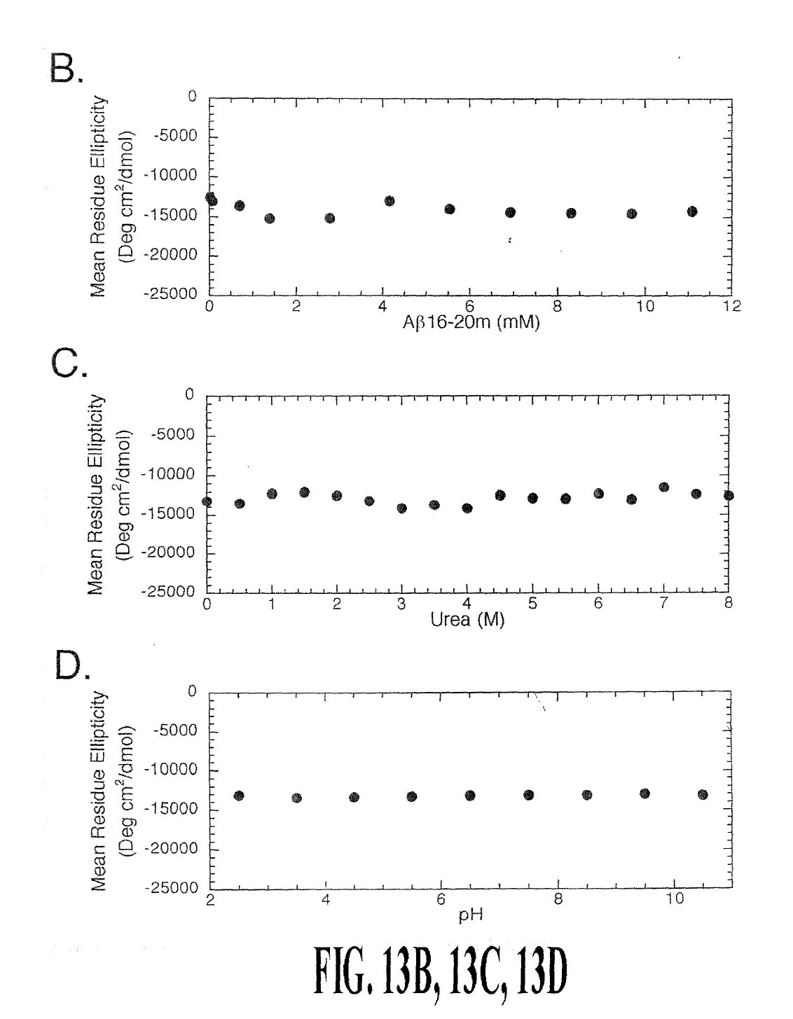

The molecular weight of Aβ16-20m was determined. Using sedimentation equilibrium analytical ultracentrifugation, a molecular weight of 537 was determined (FIG. 12A). This was close to the calculated, monomeric molecular weight of 722. The difference in molecular weights may be the result of the shallow concentration gradient established in the ultracentrifugation cell due to the low molecular weight of the peptide.

This result was confirmed by size exclusion chromatography, in which the peptide eluted from a Superdex Peptide column in a position consistent with that of a monomer (FIG. 12B). The retention time was somewhat greater than the column volume, suggesting that the peptide adsorbed to the column. The area and retention time of the peak, however, were invariant for aliquots of a single peptide sample, injected repeatedly onto the column over three days. Similar results were also obtained with a Superdex 75 column.

Data obtained from CD and NMR spectroscopy was consistent with the monomeric state of A(β16-20)ₘ over a concentration range of 0.5 to 30 mM.

### Example 11

A. Circular Dichroic and Two-Dimensional NMR Spectroscopy of Aβ16-20 and Aβ16-20 Variants

The circular dichroic (CD) spectra were recorded using a Jasco P715 spectropolarimeter.

For the concentration dependency experiment, Aβ16-20m, at concentrations ranging from 0.01 mM to 11 mM, was dissolved in 100 mM phosphate buffer at pH 7.4. A 1 mm or 0.1 mm pathlength cell was used for measurements, depending on the concentration of the solution. Six to eight scans were acquired from 250 nm to 200 nm. For the pH experiment, a 100 mM phosphate-citrate buffer was used for pH 2.5-6.5, a 100 mM phosphate buffer was used for pH 7.5-8.5 and a 100 mM glycine-NaOH buffer was used for pH 9.5-10.5. For the urea denaturation experiment, Aβ16-20m was dissolved in 100 mM phosphate buffer with the appropriate concentration of urea.

The circular dichroic spectra of Aβ16-20m, shown in FIG. 13A, resembles that of a typical β-sheet, except that the minimum is red-shifted to 226 nm. The red-shifted minimum has been observed for other (β-sheet peptides and has been attributed to the twist of the β-strand (Orpiszewski et al., 1999; Cerpa et al., 1996; Manning et al., 1988; and Zhang et al., 1997). Other peptides with N-methyl amino acids also exhibit this shifted minimum (Chitnumsub et al., 1999). In contrast to Aβ116-20m, Aβ16-20 exhibited a CD spectrum characteristic of a random coil.

FIG. 13C and FIG. 13D demonstrate that the mean residue ellipticity (226 run) of Aβ16-20m was invariant over a wide range of urea concentrations and pH values, indicating that the structure of the peptide was extremely stable and resistant to chemical denaturation. Similarly, 8M. GuHCl had no effect on the structure of the peptide, as assessed by circular dichroism. The CD spectra taken at temperatures of 20° and 70° C. were superimposible, again indicating rigidity of the structure and resistance to denaturation. Also, the MRE of Aβ16-20m was constant over 800-fold range of peptide concentrations (FIG. 13B). This was also observed for the Aβ16-22m inhibitors and suggested that the peptide did not aggregate in solution.

### Example 12

A. Nuclear Magnetic Resonance

The circular dichroism data suggested that the Aβ16-20m peptide adopted an extended, or β-strand, conformation in solution. The structure of this peptide was also investigated with 1D and 2D NMR spectroscopy.

The NMR data collection was performed as described by Benzinger et al., (1998). Briefly, NMR samples were prepared by dissolving the A 16-20m peptide in a solution of 100 mM phosphate buffer at pH 4.5 with 10% D₂0 (v/v). The 1D spectra were recorded on a 1 mM Aβ16-20m sample. The 2D spectra were collected on a 30 MM Aβ16-20m sample. The NMR experiments were performed on a Varian 600 MHz spectrometer at 15° C. Typical two dimensional data were recorded with 256 free induction decays (FIDs) of 2k data points, 16 scans per FID and a spectral width of 6000 HZ in both dimensions. Presaturation was used for water suppression, which included 2.5 s of continuous irradiation. The ROESY and TOCSY spectra were recorded with mixing times of 300 ms and 50 ms, respectively. All samples were referenced to DSS (0 ppm). Data were processed using the Varian VNMR version 6.1 software. The Φ torsional angles were estimated from the equation from Wuthrich³¹, i.e. ³J_{HNα}=6.4 cos²θ−1.4cos θ+1.9, where θ=|Φ−60|

Comparison of 1D spectra over a 30-fold concentration range did not reveal any change in peak ratios or chemical shift, again suggesting that the peptide was monomeric at all concentrations and did not aggregate. The amide protons were well dispersed over a chemical shift range of 1 ppm. The ³J_{HN} coupling constants range from 7-9 Hz and are summarized in Table 6. In general, coupling constants. 7 Hz were considered characteristic, or diagnostic, of R-strand conformations. Based on a Karplus-type relation, a range for the dihedral angle D was estimated from the coupling constant (Vitoux et al., 1986). These values were also summarized in Table 6 and range from −80 to −160°. These Φ angles were characteristic for a peptide in an extended, or R-strand, conformation.

TABLE 6
Summary of ³J_{HN} Coupling Constants and Range of Corresponding Φ
Angles

| Residue | ³J_{HN} | Φ Angle |
|---|---|---|
| Lys1 | 7.1 | −80 to −160 |
| Phe5 | 7.7 | −80 to −160 |
| Val3 | 9.2 | −80 to −160 |

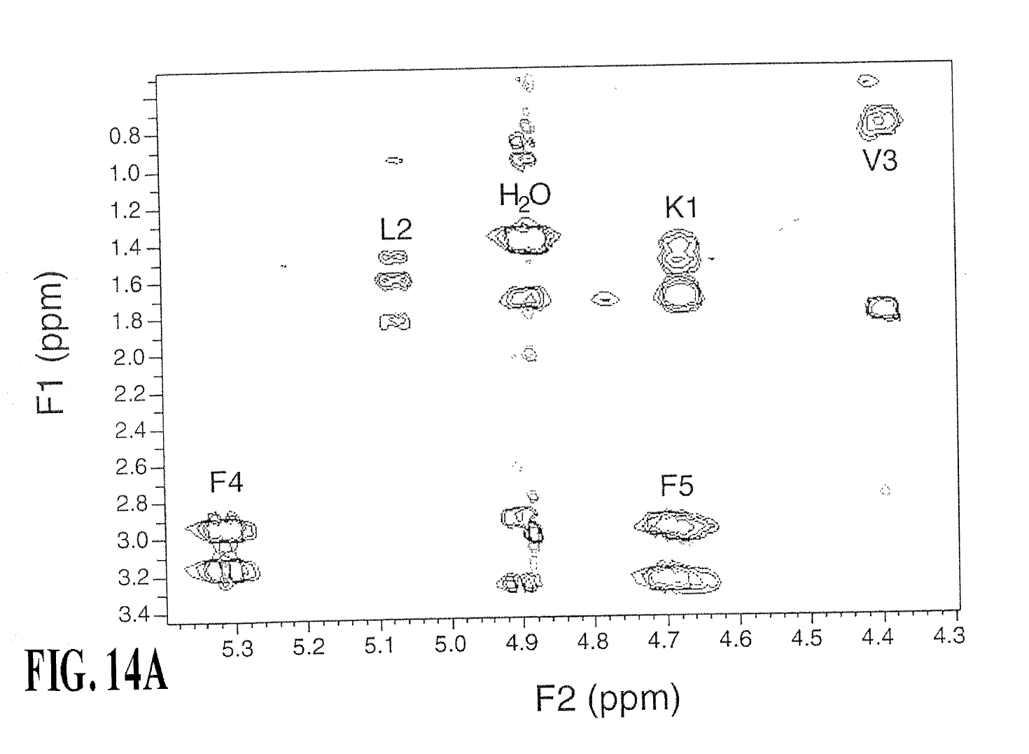

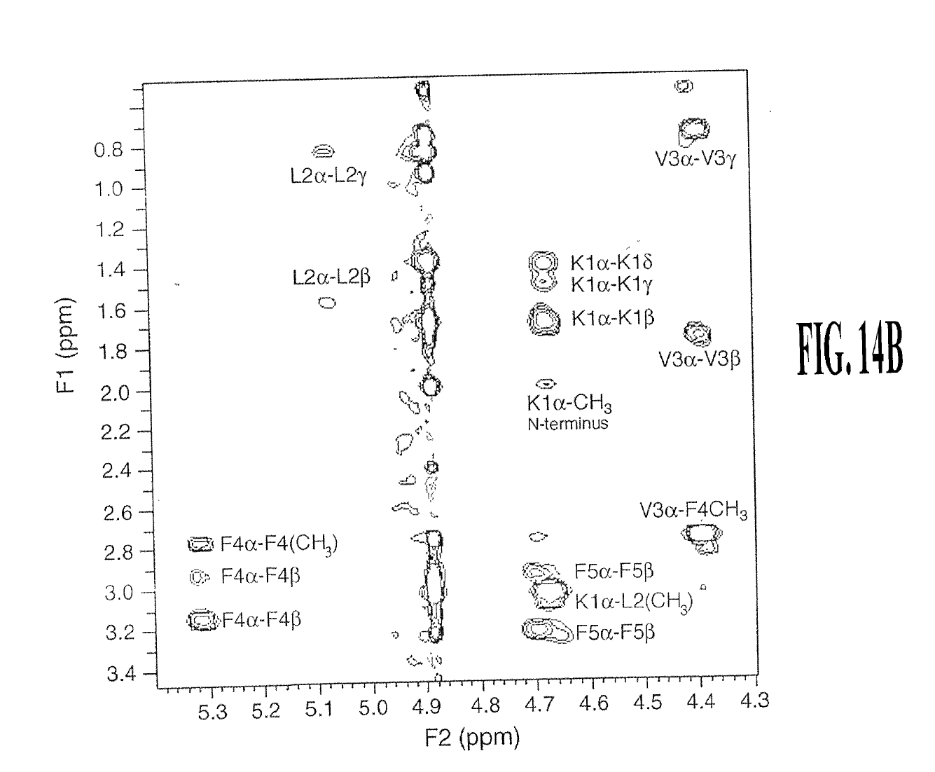

As expected for a peptide in an extended or R-strand conformation, intra-residue NOEs were almost exclusively observed in the ROESY experiment. Extensive NOEs were observed between the NH, H and sidechain protons for each residue. Inter-residue H—NCH₃ contacts were observed between Lys1 and Leu2 and Val3 and Phe4 (FIG. 14B). This pattern of inter-residue NOEs was predicted for a peptide in an extended, or R-strand, conformation.

### Example 13

A. Vesicle and Cellular Membrane Permeability

Aβ16-22m was highly soluble in aqueous media. This trait was also exhibited by Aβ16-20m and PrP115-122m, and appeared to be a general characteristic of N-methyl amino acid containing peptides.

The hydrophobicity of Aβ16-20m sequence suggested that it might be able to permeate phospholipid bilayers and cell membranes. This peptide has a single, charged lysine residue, an acetylated N-terminal and amidated C-terminal. There are also two N-methyl groups in the peptide backbone, which leaves only three amide protons vailable for hydrogen bonding. In addition, the peptide was highly soluble not only in aqueous media, but also in a variety of organic solvents including DMF, diethyl ether, methylene chloride, and chloroform. The membrane permeabilty of this peptide was tested in vitro using phosphotidylcholine vesicles and ¹⁴C-labeled Aβ16-20m.

¹⁴C-Aβ16-20m and ³H-glycine (Amersham) were dissolved in 100 mM phosphate buffer at concentrations of 5 mM and 0.5 mM, respectively. Phosphotidylcholine (Avanti Polar Lipids), dissolved in chloroform, was dried under a stream of nitrogen and then stored under vacuum overnight. The dried lipids were rehydrated with the Aβ16-20m and glycine solutions, vortexed for several minutes and subjected to five freeze/thaw cycles. The lipid suspensions were extruded through a membrane with a 100 nm pore size using a mini-extruder (Avanti Polar Lipids). The vesicles were then separated from free Aβ16-20m and glycine by passage over a G25 column (Pharmacia). The vesicle solution was incubated at 37° C. during the assay.

The efflux of radioactive material from the vesicles was monitored essentially as described by Austin et al., (1995 and 1998). Briefly, the effluxed Aβ16-20 m and glycine were separated from the vesicles by ultrafiltration through Microcon Microcentrators (Amicon) with a molecular weight cutoff of 3000. A 200 gl aliquot of the vesicle solution was spun for 20 minutes at 14000 g. The radioactivity, ¹⁴C and ³H, present in the filtrate was quantitated with scintillation counting. The total radioactivity was determined by adding 0.1% Triton X-100 to an aliquot of vesicle solution and then centrifuging. Comparison of the total radioactivity determined by this method and by sampling the vesicle solution directly, without the subsequent centrifugation step, revealed that approximately 5% of the material was retained on the filter.

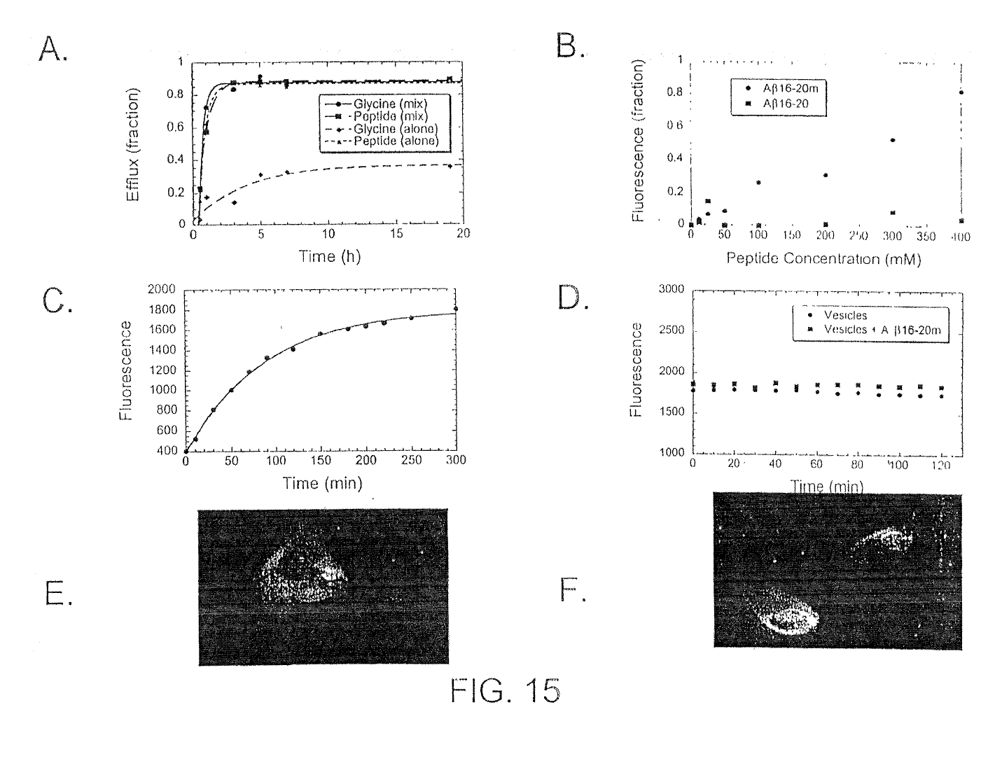

FIG. 15A demonstrates that efflux of the radioactive peptide from single bilayer lecithin vesicles is nearly 100% over a five hour period (“Peptide alone”). ³H-Glycine, a negative control for vesicle integrity, exhibits a low level of efflux over the same time period (“Glycine alone”), probably attributable to the presence of uncharged amino acid present at a low concentration at pH near neutrality. The efflux of glycine, however, increases to the level of efflux of A 16-20m when it is included in vesicles with the Aβ16-20m peptide (“Peptide (mix)” and “Glycine (mix)”). This suggests that the peptide may be changing the permeability or integrity of the vesicles.

### Example 14

A. Calcein Leakage Assay

Solubility was investigated in greater detail using a calcein leakage assay. Calcein is a fluorescent molecule that self-quenches when it is trapped in the interior of a vesicle at high concentration.

The leakage of vesicle contents was monitored by measuring the release of calcein (Terzi et al., 1995 and Pillot et al, 1996). Vesicles were prepared and separated from free calcein as described above for the radioactive compounds, except that the rehydration buffer contained β40 mM calcein and 10 mM Na-EDTA. In the kinetic assay, peptide was added to the vesicle solution and the fluorescence was measured at ten minute intervals with excitation and emission wavelengths of 490 and 520 nm, respectively. Data were fit to an equation for a first order rate process. For the concentration dependence assay, different amounts of peptide were added to the vesicle solution and the fluorescence was measured after a two hour incubation at 37° C. The maximum leakage was determined by lysing the vesicles with the addition of 0.5% (w/v) Triton X-100.

As demonstrated in FIG. 15B, Aβ16-20m caused the leakage of calcein from the interior of phosphotidylchloine vesicles. The amount of calcein efflux was linearly dependent on inhibitor concentration. At low micromolar concentrations of Aβ16-20m, less than 10% calcein efflux was observed. At β400 pM inhibitor, the highest concentration tested, 82% of the total calcein escaped from the vesicles. A kinetic analysis of the calcein leakage (FIG. 15C) demonstrated a first order rate dependence with a rate constant of 0.01 min.

### Example 15

A. Right Angle Light Scattering

The effect of Aβ16-20m on vesicle size was monitored by following the change in 90 light scattering (Pillot et al., 1996 and Lu et al., 2000). Vesicles were prepared as described in the previous example. The 90° light scattering of vesicle solutions in the presence or absence of peptide were measured on a Hitachi F-2000 spectrofluorimeter with both the excitation and emission wavelengths set to 600 nm.

Right angle light scattering (FIG. 15D) did not indicate any difference in the size of vesicles in the presence or absence of Aβ16-20m. This suggested the inhibitor does not cause the reorganization or fusion of lipid vesicles. The fact that efflux of 3H-glycine increased dramatically in the presence of A(β16-20)ₘ, and the fact that Aβ16-20m does not cause fusion of vesicles, together suggested that the N-methyl peptides create minute, transient pores in the bilayer, through which peptide and ³H-glycine can pass from the included solvent to the bulk solvent, but which seal rapidly, leaving the bilayer intact.

### Example 16

A. Cell Assays

The vesicle assays with the Aβ16-20m peptide demonstrated in vitro vesicle permeability. To facilitate in vivo and cellular experiments, the A 16-20m peptide was prepared with a fluorescent probe, N-methyl anthranilic acid (Jureus et al., 1998), at the N-terminal. The fluorescent peptide, Anth-Aβ16-20m, was incubated with COS cells for twelve hours.

Briefly, COS cells, were plated on coverslips, and incubated overnight in the presence of 4 μM to β40 μM of the Anth-A16-20 peptide. The cells on coverslips were then washed extensively with PBS, fixed for one hour with a 3.7% formaldehyde solution and mounted on a slide. The cells were examined by fluorescence microscopy using a DAPI filter.

FIG. 15E and FIG. 15F show COS cells incubated with different concentrations of Anth-Aβ16-20. Strong fluorescence was observed at peptide concentrations of 20 μM (FIG. 15E) and pM (FIG. 15F). Very weak fluorescence was observed at peptide levels below 4 μM. These results clearly demonstrated that the Anth-Aβ16-20 peptide was permeable to cell membrane. The Aβ16-20m peptide, based on the vesicle data and its structural similarities to Anth-Aβ16-20m, is also most likely permeable to cell membranes.

### Example 22

A. In Vivo Studies

The Aβ16-22m, A-β16-22mR, Aβ16-20m and Anth-Aβ16-20m, peptides were as effective or more effective than any other inhibitor of fibrillogenesis reported previously; moreover, they were also effective or more effective at disassembling pre-formed fibrils of A. Thus, these peptides provide prototypes of a new class of therapeutic agents for Alzheimer's disease. The inventors envision translating these findings in vitro into the clinical arena. First, as with any potential therapeutic agent, the toxicity, especially neurotoxicity of these peptides will be assessed. Second, biodistribution of the peptides, and their ability to cross the blood-brain barrier will be evaluated. In this connection, it has been shown that even Aβ40 itself can cross the blood brain barrier (Saito et al., 1995; Pluta et al., 1996; Poduslo et al., 1999; Strazielle et al., 2000). Furthermore, both the high water solubility and the increased hydrophobicity of N-methyl peptides (compared to non-methylated congeners) indicate an ability to cross the blood-brain barrier. Indeed, Burton et al (1996) have provided evidence, from water-organic solvent partitioning, direct observation of passage of peptide through Caco-2 cell membranes, and transport across a parietal cerebrovascular permeability barrier, that N-methyl peptides can cross these divides more readily than their non-methylated counterparts. Third, the inventors will determine whether sufficient levels of inhibitor peptides required for therapy can be reached and then sustained in the central nervous system over a period of time to affect the course of diseases that involve fibril formation such as to name a few examples, Alzheimer's Disease, Down's Syndrome, Dutch-Type Hereditary Cerebral Hemorrhage Amyloidosis, Reactive Amyloidosis, Familial Mediterranean Fever, Familial Amyloid Nephropathy With Urticaria And Deafness; Muckle-Wells Syndrome, Idiopathic Myeloma, Macroglobulinemia-Associated Myeloma, Familial Amyloid Polyneuropathy, Familial Amyloid. Cardiomyopathy, Isolated Cardiac Amyloid, Systemic Senile Amyloidosis, Adult Onset Diabetes, Insulinoma, Isolated Atrial Amyloid, Medullary Carcinoma Of The Thyroid, Familial Amyloidosis, Hereditary Cerebral Hemorrhage With Amyloidosis, Familial Amyloidotic Polyneuropathy, Scrapie, Creutzfeldt-Jacob Disease, Gerstmann-Straussler-Scheinker Syndrome, Bovine Spongiform Encephalitis, Prion mediated disease, Huntington's Disease.

Thus, the development of pharmaceuticals based on the peptides of the invention that can not only prevent, but even reverse the formation of fibrils will be important in therapy of diseases that involve fibril formation such as those listed above. This would seem especially true of early Alzheimer's Disease or early stages of any of the above dosages, where a goal would be to prevent or reverse ongoing neural damage from nascent fibrils or their immediate precursors. Furthermore, the strategy of using N-methyl amino acids in inhibitor peptides may be applicable to other diseases that involve aberrant protein. aggregation, and can, therefore, be applied to any self-associating proteins for which a site of peptide-peptide interaction is known. Preliminary studies are also underway which indicate that an N-methyl amino acid-containing peptide directed at aggregation of the prion protein be an effective aggregation inhibitor. Thus, the inventors envision that the peptide inhibitors described herein offer therapeutic benefit in Alzheimer's Disease, Prion Disease, Huntingtons, a host of other amyloid diseases and the other diseases listed above.

(a) Animal Models

Animal models may be used to test the effect of the polypeptides of the present invention before a human clinical trial. Preferably, orthotropic animal models will be used so as to closely mimic the particular fibril disease type being studied and to provide the most relevant results.

One type of orthotropic model involves the development of an animal model for the analysis of fibril associated pathologies. Virtually any animal may be employed, however, for use according to the present invention. Particularly preferred animals will be small mammals that are routinely used in laboratory protocols. Even more preferred animals will be those of the rodent group, such as mice, rats, guinea pigs and hamsters. Rabbits also are a preferred species. The criteria for choosing an animal will be largely dependent upon the particular preference of an investigator.

Induction of an experimental fibril based pathology is the first step. Although establishing an optimal model system for any particular type of fibril based pathology may require a certain adjustment in the amount of fibril forming protein administered to the animal, this in no way represents an undue amount of experimentation. Those skilled in the area of animal testing will appreciate that such optimization is required.

In one example, induction of experimental amyloidosis may be performed as previously described (LeVine et al., 1993; Snow et al., 1991). BALB/c mice can be injected t.v. with 100 μg of amyloid enhancing factor (AEF) alone or preincubated for 24 h with 5 mg of β-amyloid. AEF can be prepared using the standard protocols (Merlini et al., 1995). The AEF injection will be followed by a single s.c. injection of 0.5 ml of 2% silver nitrate. Animals are then sacrificed 5 days after the injection and the amyloid quantitated by immunohistochemistry and congo red staining. A standard set of amyloid containing tissue is generated (5%, 10%, 20%, 30%, p40%, 50). These were reference points to determine the amount of amyloid in a given tissue. Standard sections were examined under the microscope (Nikon, using polarizing filters to generate birefringence for Congo red). The images can be digitized and analyzed by computer.

One may then experiment with the polypeptides of this invention to study how the peptides inhibit and/or disassemble fibril formation. The skilled artisan will readily be able to adapt or modify each particular model for his intended purpose without undue experimentation.

(b) Clinical Diagnosis

To this date, there is no feasible diagnostic procedures to diagnosis a patient with Alzheimer's Disease, except by autopsy. Thus, the inventors have contemplated that the present invention may be used to develop a diagnostic test. It is envisioned that administration of the polypeptide inhibitors of the present invention may congregate and adhere to the tangles or fibrils that are formed in the brain.

In the diagnostic test, it is contemplated that the polypeptide inhibitor sequences of the present invention may be conjugated to a marker for detection, i.e., radiolabel or a other radiographic contrasting agents. Examples of the polypeptide sequences that are contemplated in the present invention include, but are not limited to (Aβ16-22): NH₂—KLVFFAE-CONH₂; (Aβ16-22m): NH₂—K(me-L)V(me-F)F(me-A)-E-CONH₂; (Aβ16-22mR): NH₂-E(me-L)V(me-25 F)F(me-A)—K—CONH₂; (Aβ16-22m(4)): NH₂—KL(me-V)(me-F)(me-F)(me-A)-E-CONH₂; (Aβ16-20m): Ac—NH—K(me-L)V(me-F)F—CONH₂; (Anth-Aβ16-20m): Anth-NH—K(me-L)V(me-F)F—CONH₂; (Aβ16-20R): Ac—NH—KLredVFredF—CONH₂; (Aβ16-20: EAc—NH—K_{Lester}, V_{Fester} F—CONH₂; (Ac-Aβ16-22): Ac—NH—KLVFF—CONH₂; and (AD 1-Aβ40): NH₂-DAEFRHDSGYEVHHQKLVFFAEDVGSNKGAIIG LMVGGVVIA-COOH.

These sequences are conjugated to marker by methods well known and used in the art. The imaging moieties used can be paramagnetic ions; radioactive isotopes; fluorochromes; NMR-detectable substances; and X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (II1), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

Radioactive isotopes for therapeutic and/or diagnostic application may include, but are not limited to astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, phosphorus, rhenium¹⁸⁶, rhenium¹⁸⁸, ⁷⁵selenium, ³⁵sulphur, technicium^{99m} and/or yttrium^{90m}. ¹²⁵I is often being preferred for use in certain embodiments, and technicium⁹⁹m and/or indiums¹¹¹ are also often preferred due to their low energy and suitability for long range detection.

Other radiographic contrasting agents may be used for example, barium, gastrograffin or galalidium.

It is envisioned that the conjugated polypeptides may be administered orally or systemically, i.e., intravenously. Once administered, the patient can be examined using a variety of radiographic instruments, for example, X-ray, MRI or CAT scan.

(c) Clinical Trials

This example is concerned with the development of human treatment protocols using the polypeptides of the invention that inhibit fibril formation and disassemble pre-formed fibrils. These polypeptide compositions will be of use in the clinical treatment of various fibril based diseases caused by fibril formation and deposition of fibrils in cells and tissues. Such treatment will be particularly useful tools in treating diseases such as Alzheimer's Disease, Down's Syndrome, Dutch-Type Hereditary Cerebral Hemorrhage Amyloidosis, Reactive Amyloidosis, Familial Mediterranean Fever, Familial Amyloid Nephropathy With Urticaria And Deafness, Muckle-Wells Syndrome, Idiopathic Myeloma, Macroglobulinemia-Associated Myeloma, Familial Amyloid Polyneuropathy, Familial Amyloid Cardiomyopathy, Isolated Cardiac Amyloid, Systemic Senile Amyloidosis, Adult Onset Diabetes, Insulinoma, Isolated Atrial Amyloid, Medullary Carcinoma Of The Thyroid, Familial Amyloidosis, Hereditary Cerebral Hemorrhage With Amyloidosis, Familial Amyloidosic Polyneuropathy, Scrapie, Creutzfeldt-Jacob Disease, Gerstmann-Straussler-Scheinker Syndrome, Bovine Spongiform Encephalitis, Prion-mediated diseases, Huntington's Disease.

The various elements of conducting a clinical trial, including patient treatment and monitoring, will be known to those of skill in the art in light of the present disclosure. The following information is being presented as a general guideline for use in establishing polypeptide compositions described herein alone or in combinations with other drugs used routinely in fibril based diseases in clinical trials.

Candidates for the phase 1 clinical trial will be patients on which all conventional therapies have failed. Polypeptide compositions described herein will be administered to them regionally on a tentative weekly basis. The modes of administration may be among others endoscopic, intratracheal, percutaneous, or subcutaneous. To monitor disease course and evaluate the inhibition of fibril formation and/or disassembly of fibrils, it is contemplated that the patients should be examined for appropriate tests every month. Tests that will be used to monitor the progress of the patients and the effectiveness of the treatments include: physical exam, X-ray, blood work and other clinical laboratory methodologies. The doses given in the phase 1 study will be escalated as is done in standard phase 1 clinical phase trials, i.e. doses will be escalated until maximal tolerable ranges are reached.

Clinical responses may be defined by acceptable measure. For example, a complete response may be defined by complete disappearance of evidence of fibrils for at least 2 months. Whereas a partial response may be defined by a 50% reduction of fibrils and their deposits for at least 2 months.

The typical course of treatment will vary depending upon the individual patient and disease being treated in ways known to those of skill in the art. For example, a patient with amyloidosis might be treated in eight week cycles, although longer duration may be used if no adverse effects are observed with the patient, and shorter terms of treatment may result if the patient does not tolerate the treatment as hoped. Each cycle will consist of between 20 and 35 individual doses spaced equally, although this too may be varied depending on the clinical situation.

Optimally the patient will exhibit adequate bone marrow function (defined as peripheral absolute granulocyte count of >2,000/mm3 and platelet count of 100, 000/mm³, adequate liver function (bilirubin 1.5 mg/dl) and adequate renal function (creatinine 1.5 mg/dl).

A typical treatment course may comprise about six doses delivered over a 7 to 21 day period. Upon election by the clinician the regimen may be continued with six doses every three weeks or on a less frequent (monthly, bimonthly, quarterly etc.) basis. Of course, these are only exemplary times for treatment, and the skilled practitioner will readily recognize that many other time-courses are possible.

Thus the present invention provides effective peptide inhibitors of fibrillogenesis. The inventors also envision that these peptides may be used as potential structural probes of Aβ fibrillogenesis. The inventors contemplate examining the mode of association between these inhibitor peptides and Aβ40, as well as the structure and pharmacodynamics of the inhibitor peptides themselves with the goal of developing effective pharmaceuticals to combat fibrillogenesis.

### Example 18

A. Design and Characterization of a Membrane Permeable N-Methyl Amino Acid-Containing Peptide That Inhibits Aβ1-40 Fibrillogenesis

(a) Peptides:

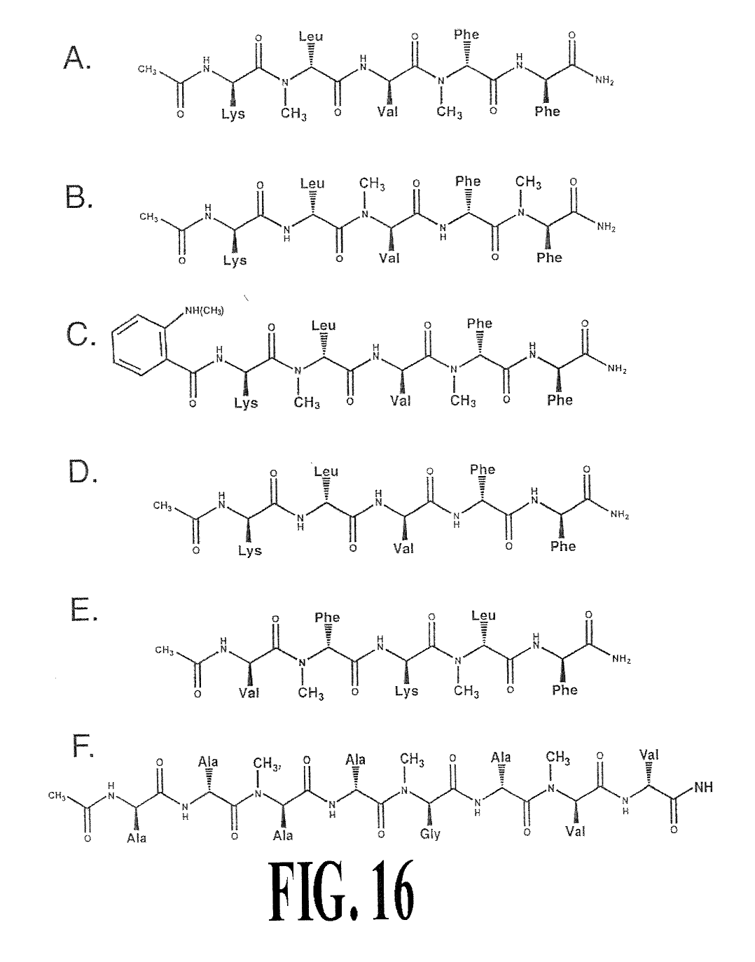

The Aβ16-20m peptide (FIG. 16) resembles Aβ1-40 fibrillogenesis, peptide Aβ16-22m. Both Aβ16-20m and Aβ16-22m are homologous to the central region of Aβ (residues 16-22) and contain alternating methyl groups, which are designed to inhibit Aβ fibrillogenesis and disassemble pre-formed fibrils Aβ16-20m was designed so that, as a 5-strand, it would present one “face” that can form hydrogen bonds with A3A peptides, but a second “face” in which the ability to form hydrogen bonds is severely reduced through the replacement of amide hydrogens by methyl groups. To determine whether a fibrillogensis inhibitor would permeate through natural and synthetic phospholipid bilayer membranes, the Aβ16-20m peptide was truncated (with respect to Aβ16-22m) in order to eliminate a charged residue (Glu) and to give the inhibitor a net positive charge, a trait found in other membrane permeant peptides.

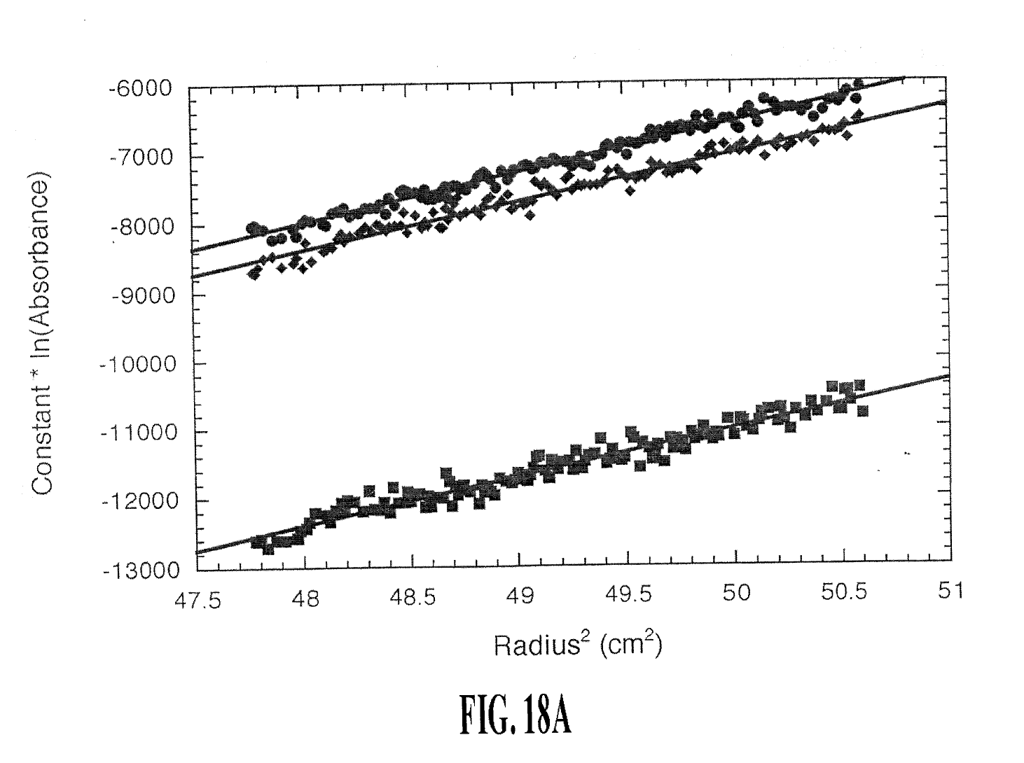

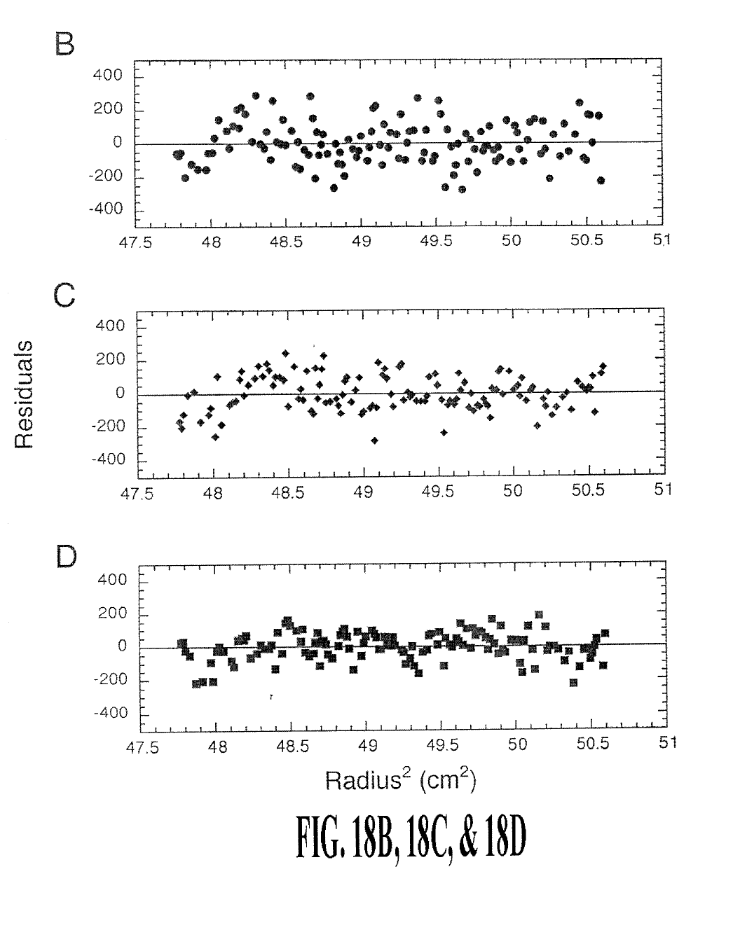

A number of relevant congeners of Aβ16-20m are shown in FIG. 18A. The Aβ16-20 m2 peptide (FIG. 16B) is identical to the Aβ16-20m peptide except the positions of the N-methyl groups are shifted; N-methyl amino acids are incorporated at residues 18 and 20, rather than 17 and 19. The alternating pattern of the N-methyl groups, however, is maintained in the Aβ16-20n2 peptide. Aβ16-20m was also labeled at the α-amino group with the fluorescent probe N-methyl anthranilic acid to create the Anth-Aβ16-20m peptide (FIG. 16C). The Aβ16-20 peptide (FIG. 16D) was synthesized as a positive control because another group has demonstrated that it is an effective inhibitor of β-amyloid fibrillogenesis (Tjernberg et al., 1997). Finally, to test the sequence specificity of the N-methylated inhibitor peptides, a peptide, Aβ16-20s, was synthesized (FIG. 16E), that was identical to Aβ16-20m except that the order of the amino acids was scrambled. As a further test of sequence specificity, PrP115-122m was synthesized (FIG. 16F), which has N-methyl amino acids in alternate positions. PrP115-122m, as demonstrated herein, inhibits the aggregation of a peptide, PrP106-126, derived from the human prion protein.

N-methyl amino acid-containing peptides were synthesized with excellent purity using HATU activation for residues after N-methyl amino acids (Coste et al., 1990, 1991; Carpino, 1993; Carpino et al., 1994). N-methyl anthranilic acid was treated as a normal amino acid and coupled using HBTU/HOBt chemistry without protection of the secondary amine.

(b) Inhibition of Fibrillogenesis and Disassembly of Pre-formed Fibrils:

Electron microscopy and thioflavin assays were performed to assess the fibril inhibition and disassembly activities of the new inhibitor peptides. In inhibition assays, samples without inhibitor peptide demonstrated long, unbranched fibrils by electron microscopy (FIG. 8A), while samples containing Aβ16-20m did not demonstrate any fibrillar material, although some amorphous precipitate was observed (FIG. 8B). Some fibrillar material was observed in samples containing the Aβ16-20 peptide (FIG. 8C). The Aβ16-20 peptide, however, forms fibrils (FIG. 8D) on its own and it is not clear if the fibrils observed by electron microscopy are composed of Aβ16-20, Aβ31-40 or a mixture of both peptides.

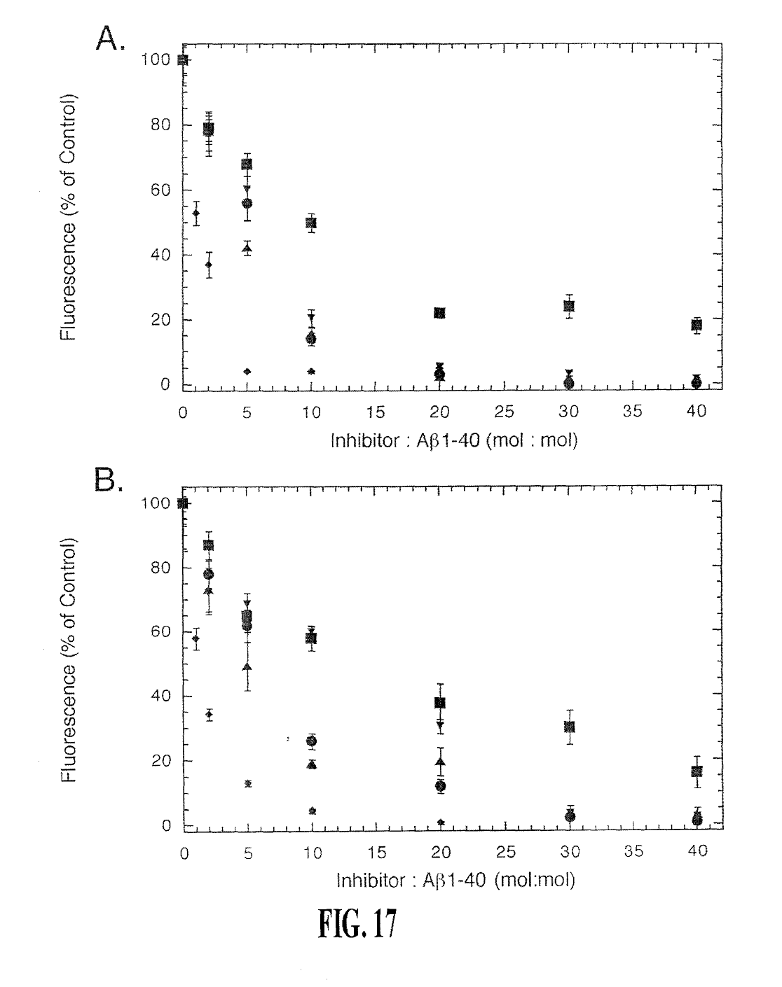

Thioflavin T fluorescence assays (FIGS. 17A and B) demonstrated that Aβ16-20m is an effective fibrillogenesis inhibitor, and also disassembles pre-formed Aβ1-40 fibrils, more so than the non-methylated congener Aβ16-20. None of these inhibitor peptides demonstrate any thioflavin fluorescence in the absence of Aβ1-40 peptide. In particular, although Aβ16-20 forms fibrils (FIG. 8D) and binds Congo red, it does not cause thioflavin fluorescence. Table 9 summarizes the ICₘₐₓ and IC₅₀ parameters obtained from least squares fit of the data to the equation of a hyperbola (see Materials and Methods). As with fibril inhibition assays, electron microscopy of samples containing Aβ1-40 fibrils incubated with Aβ16-20m for three days (FIG. 8B) showed no fibrillar material. Fibrillar material was observed, however, when the Aβ1-40 peptide was incubated with A 16-20 (FIG. 8E) in a disassembly assay, the difference between Aβ16-20 and Aβ16-20m being more marked in fibril disassembly than inhibition assays. In general, assessment of fibril disassembly using thioflavin T fluorescence was in agreement with results obtained from electron microscopy (Table 9 and FIG. 17B). The kinetics of fibril disassembly were best fit by a pseudofirst-order rate law, with a half-life for disassembly, calculated from the pseudofirst-order rate constants, of 24±7 min. The rate constant showed little variation with inhibitor peptide concentration.

(c) Equilibrium Analytical Ultracentrifugation:

For NMR and other studies, it was important to assess the degree of self-association, if any, of the inhibitor peptide Aβ16-20m. The molecular weight of Aβ16-20m in solution was measured using equilibrium analytical ultracentrifugation (FIG. 18). Data were collected at three different rotor speeds. Modeling the data as a single ideal species resulted in the best agreement with the theoretical curves. A molecular weight of 695±27 was measured for a 1 mM sample of Aβ16-20m. The calculated molecular weight of Ad 16-20m is 722.78, which suggests that Ad 16-20m is monomeric in solution. Data presented herein, below, from CD and NMR spectroscopy are consistent with the monomeric state of Aβ16-20m over a concentration range of 0.5 to 30 mM. Analytical ultracentrifugation data were not obtained for Aβ16-20 because this peptide forms aggregates, or fibrils, that pellet even at low centrifugation speeds.

(d) Circular Dichroism and Two-Dimensional NMR Spectroscopy:

The circular dichroic spectrum of Aβ16-20m, shown in FIG. 5A, resembles that of a β-sheet, except that the minimum is red-shifted to 226 nm from the canonical 217 nm. (Other investigators have reported that N-methyl amino acids induce β-sheet structure in peptides [Tjernberg et al., 1997; Tonelli, 1970, 1971, 1974; Vitoux et al., 1986; Kumar et al., 1975]).

FIGS. 5C and 5D demonstrate that the mean residue ellipticity (MRE) at 226 nm of Aβ16-20m is invariant over a wide range of urea concentrations and pH values, indicating that the structure of the peptide is extremely stable and resistant to chemical denaturation. Similarly, 8M GuHCl had no effect on the structure of the peptide, as assessed by circular dichroism. The CD spectra taken at temperatures of 20° and 70° C. were superimposible, again indicating the rigidity of the structure and resistance to denaturation. Also, the MRE of Aβ16-20m is constant over an 800-fold range of peptide concentrations (FIG. 5B). This was also observed for the Aβ16-22m inhibitors and suggests that the peptide does not aggregate in solution.

The circular dichroism data suggest that the Aβ16-20m peptide adopts an extended, or β-strand, conformation in solution. The structure of Aβ16-20m was investigated using 1D and 2D NMR spectroscopy. Comparison of 1D spectra of Aβ16-20m over a 30-fold concentration range (1 mM to 30 mM) did not reveal any change in peak ratios or chemical shifts, again suggesting that the peptide is monomeric. The ³J_{HNα} coupling constants range from 7-9 Hz and are summarized in Table 8. In general, coupling constants >7 Hz are considered characteristic, or diagnostic, of β-strand conformations. Based on a Karplus-type relation the dihedral angle φ was estimated from the coupling constant (Wuthrich, 1986). The measured J-values are large enough that two, rather than four, φ-values fulfill the Karplus equation. The smaller of the two φ angles range from −82° to −104°, while the larger angles range from −132.4° to −157°. The larger φ angles are consistent with a peptide in an extended, or β-strand, conformation. The canonical 4 values for parallel and antiparallel β-sheets, for example, are −119° and −139°, respectively. These angles are significantly larger than those observed in a canonical α-helix or 3₁₀-helix, −57° and −60°, respectively. Although large J-values are also observed for residues in random coil conformations, CD spectra do not support this conformation, and are consistent with the interpretation of a β-strand structure.

The α protons resonate between approximately 4.4. and 5.3 ppm. As demonstrated in the TOCSY spectrum (FIG. 6A), the α protons of the two N-methyl amino acids, Leu2 and Phe4, are shifted downfield to approximately 5.2 ppm (FIG. 6B). Notably, a single peak is also observed for each a proton. In other reports, N-methyl peptides with a mixture of cis and trans amide bond configurations demonstrated two peaks for each a proton (53-55). The large ³J_{NHα} values and the circular dichroism data suggest that the peptide adopts a trans, rather than cis, conformation.

As expected for a peptide in an extended or Nostrand conformation, the ROESY experiment showed almost exclusively intraresidue nOes. Extensive nOes were observed between the NH, Hα and sidechain protons for each residue. Interresidue Hα1-N(CH₃)i+1 nOes were observed between Lysl and N-methyl-Leu2 and Val3 and N-methyl-Phe4 (FIG. 6B). This pattern of interresidue nOes is predicted for a peptide in an extended, or β-strand, conformation. Although this pattern is also consistent with a random coil conformation, the circular dichroism data support the interpretation that the peptide adopts an extended, D-strand conformation in solution.

(e) Vesicle and Cellular Membrane Permeability:

Aβ16-22m, Aβ16-20m, Aβ16-20 m2, Aβ16-20s and PrP115-122m are highly soluble in aqueous media. This result is somewhat surprising in view of the fact that the methylated Aβ peptides are composed of hydrophobic residues, with the exception of a single lysine amino acid in the β-amyloid peptides, and PrP115-122m has no charged residues. In addition, two amide protons in each peptide are replaced by aliphatic methyl groups. Although the N-methyl peptides are soluble at concentrations in excess of 30 mM, the non-methylated peptide, Aβ16-20, dissolves in aqueous media at a maximum concentration of approximately 1 mM.

Despite the surprising water solubility of these peptides, they are also highly soluble in a wide variety of organic solvents as well, as might be expected for a pepide containing mainly lipophilic amino acids. This peptide has a single, charged lysine residue, an acetylated N-terminal and amidated C-terminal. In synthesizing the peptide, the highly unusual event was observed that the peptide, newly cleaved from the resin, did not precipitate in cold diethyl ether. This observation was extended and showed that the peptide was soluble to concentrations of 30 mM not only in aqueous media, but also in DMF, diethyl ether, methylene chloride, and chloroform.

The hydrophobicity of the Aβ16-20m sequence and the solubility of this peptide in both water and organic solvents suggested that it might be able to permeate phospholipid bilayers and cell membranes. The membrane permeabilty of this peptide was tested in vitro using phosphatidylcholine vesicles and ¹⁴C-labeled Aβ16-20m. Phosphatidylcholine vesicles of 100 nm diameter were prepared in the presence of radioactive Aβ16-20m. Free peptide was separated from the vesicles by passage over a PD-10 Sephadex G-25 column. The efflux of peptide from the vesicles was then monitored by an ultrafiltration assay and scintillation counting.

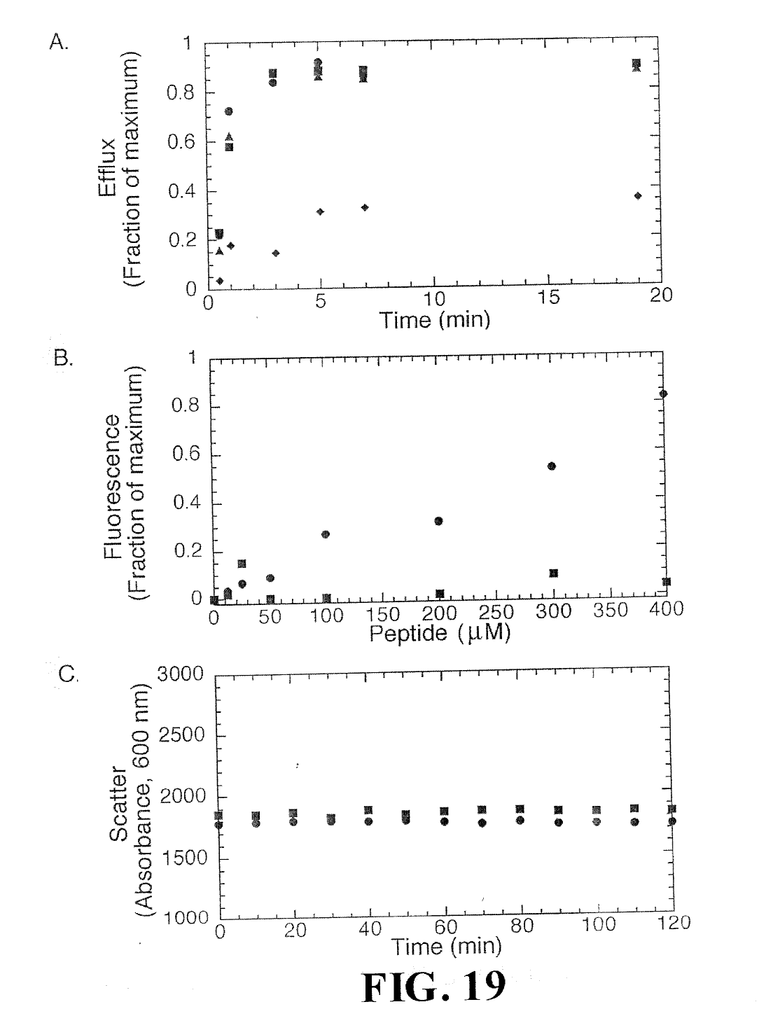

FIG. 19A demonstrates that efflux of the radioactive peptide from single bilayer lecithin vesicles is nearly 100% over a five hour period. ³H-Glycine, a negative control for vesicle integrity, exhibits a low level of efflux over the same time period, probably attributable to the presence of uncharged amino acid present at a low concentration at pH near neutrality. The efflux of glycine, however, increases to the level of efflux of Aβ16-20m when it is included in vesicles with the Aβ16-20m peptide. This observation was investigated in greater detail using a calcein leakage assay. Calcein is a fluorescent molecule that self-quenches when it is trapped in the interior of a vesicle at high concentration. Leakage of calcein from the vesicle, however, results in greatly enhanced fluorescence. As demonstrated in FIG. 19B, Aβ16-20m, but not Aβ16-20, causes the leakage of calcein from the interior of phosphatidylcholine vesicles. The amount of calcein efflux is linearly dependent on Aβ16-20m concentration. At low micromolar concentrations of Aβ16-20m, less than 10% calcein efflux is observed. At 400 sM inhibitor, the highest concentration tested, 82% of the total calcein escapes from the vesicles. Right angle light scattering (FIG. 19C) does not indicate any difference or change in the size of vesicles in the presence or absence of Aβ16-20m. This suggests the inhibitor does not cause the reorganization or fusion of the lipid vesicles.

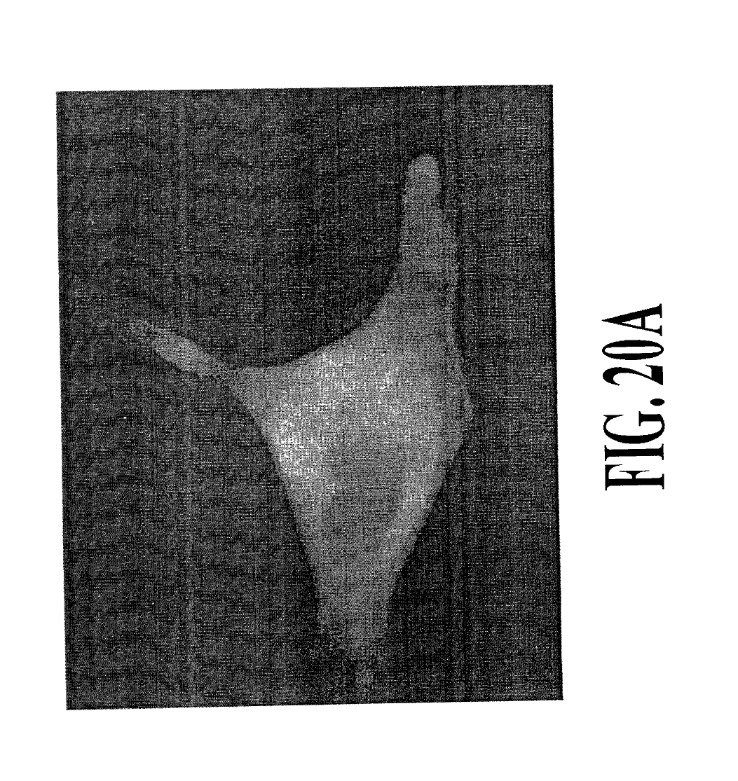

The vesicle assays with the Aβ16-20m peptide demonstrate vesicle permeability in vitro. To facilitate in vivo and cellular experiments, the Aβ16-20m peptide was prepared with a fluorescent probe, N-methyl anthranilic acid, at the N-terminal. As additional controls, Anth-Aβ16-20 (i.e., the non-methylated peptide with N-methyl anthranilic acid attached to its N-terminus) and Anth-PrP 15-122m (the analogue of the peptide shown in FIG. 16E with N-methyl anthranilic acid attached to its N-terminus), were synthesized. The fluorescent peptides were incubated with COS cells for twelve hours. The cells were then washed, fixed and examined by fluorescence microscopy. FIG. 20A shows COS cells incubated with 40 pM Anth-Aβ16-20m. Very weak fluorescence is observed at peptide levels below 4 RM. No intracellular fluorescence was observed with the other two peptides, Anth-Aβ16-20 or Anth-PrP115-122m. These results clearly demonstrate that the Anth-Aβ16-20m peptide permeates cell membranes. The Aβ16-20m peptide, based on the vesicle data and its structural similarities to Anth-Aβ16-20m, also most likely permeates cell membranes.

In order to ensure that the cellular fluorescence was not attributable to hydrolyzed (proteolyzed) Anth-Aβ16-20m, Anth-Aβ16-20m that had been internalized by COS cells after an overnight incubation was isolated. FIG. 20B is an HPLC chromatogram of the Anth-Aβ16-20m peptide before it was incubated with the COS cells. After an overnight incubation, the cells were collected and washed extensively with media until the washes did not exhibit any fluorescence due to N-methyl anthranilic acid. The cells were then lysed and the lysate was analyzed by HPLC. Fractions were collected and the Anth-Aβ16-20m peptide was detected by fluorescence spectroscopy (FIG. 20C). The N-methyl anthranilic acid-labeled peptide isolated from the COS cells elutes with the same retention time as the Anth-Aβ16-20m peptide standard, demonstrating that the internalized Anth-Aβ16-20m peptide is not modified or degraded. These results are consistent with the observation that Aβ16-22m is resistant to protease digestion by chymotrypsin in in vitro assays.

(f) Sequence Specificity:

The ability of Aβ16-20m to dissolve in organic solvents and pass through membrane raises a question about their specificity as either structural probes or potential therapeutic agents. It is possible, a priori, that these peptides operate through fairly non-specific properties, e.g., as detergents. Accordingly, the sequence specificity of these fibrillogenesis inhibitors was determined. FIG. 9 demonstrates the amino acid sequence specificity of the N-methyl amino acid-containing inhibitors in both fibrillogenesis inhibition and fibril disassembly. To investigate sequence specificity, a peptide was synthesized consisting of amino acids 106-126 of the human prion protein (Prp106-126), a peptide previously reported to form fibrils associated with thioflavin fluorescence. The peptide PrP115-122m shown in FIG. 16E, designed to inhibit fibril formation by PrP106-126, was also synthesized. Similar to Aβ16-20m, PrP 115-122m contains N-methyl amino acids at alternate residues and has an amino acid sequence derived from the central region of the peptide of which it is designed to inhibit fibril formation. As shown in FIG. 9, PrP115-122m is an effective inhibitor of fibril formation by PrP106-126, but is ineffective at inhibiting fibril formation by Aβ1-40. Similar results were obtained for fibril disassembly. By the same token, Aβ16-20m, reported herein to be an effective inhibitor of Aβ1-40 fibrillogenesis, was ineffective as an inhibitor of PrP106-126 fibrillogenesis. These data are consistent with the notion that inhibition of fibrillogenesis by N-methyl amino acid-containing peptides does require a degree of sequence homology, although amino acid composition is also clearly important.

Aβ16-20s, a scrambled version of Aβ16-20m, was also synthesized. Aβ16-20s does inhibit AN 1-40 fibrillogenesis and disassembles pre-formed Aβ1-40 fibrils. However, that with the exception of the lysine residue, Aβ16-20m is composed of entirely hydrophobic amino acids, including two phenylalanine residues. Thus, even the scrambled Aβ16-20s peptide is relatively similar to the parent Aβ16-20m peptide. These results suggest that while the inhibitor peptides are somewhat specific for amino acid sequence, the specificity is not absolute.

### Example 19

A. Inhibition of β-Amyloid (40) Fibrillogenesis and Disassembly of β-Amyloid(40) Fibrils by Short β-Amyloid Congeners Containing N-Methyl Amino Acids at Alternate Residues

(a) Design of Fibrillogenesis Inhibitor Peptides

The design of this peptide is based on two salient features of Aβ fibrils:

First, the design of the inhibitor is based on the model of the fibrillogenesis process as consisting of nucleation followed by growth—a process reminiscent of crystal nucleation and growth. Accordingly, a rationally designed inhibitor of fibrillogenesis would bind to the fibril growth sites, and thereby prevent propagation of the fibril. Ideally, the inhibitor would also distort or disrupt fibril nuclei. Since, for many ordered supramolecular aggregates, nucleation and growth are reversible processes, an ideal inhibitor would also disassemble Aβ fibrils. Two additional desirable characteristics of a pharmalogically useful fibrillogenesis inhibitor would be high water solubility, and resistance to proteases or other degradative enzymes.

Second, the design of the inhibitor is based on structural model of the Aβ fibril as laminated β-sheets. The design of the inhibitor does not rest on an assumption that fibrils contail parallel β-sheets. The peptides described below are based on the central hydrophobic “core domain,” believed to be critical in fibrillogenesis, as alteration of this domain abrogates fibrillogenesis. The strategy was to incorporate N-methyl amino acids into alternate positions of a short peptide based on the central hydrophobic core domain. In β-sheet, alternate amide protons and carbonyl oxygens are oriented to opposite sides of the peptide backbone. Thus, a peptide containing an alternation of ordinary amino acids and N-methyl amino acids should have one “face” containing ordinary amino acids, and one “face” containing N-methyl amino acids. The face containing ordinary amino acids interacts with AO in a fibril or nucleus, while the face containing N-methyl amino acids would not interact, and would, on the contrary, disrupt forming and/or existing Aβ fibrils.

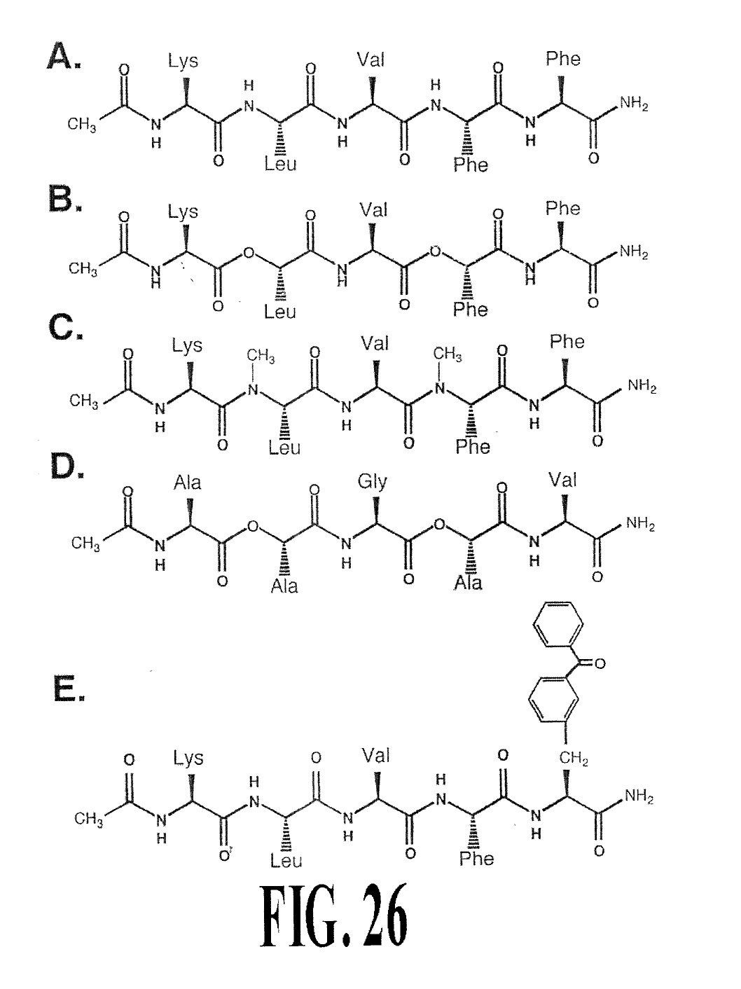

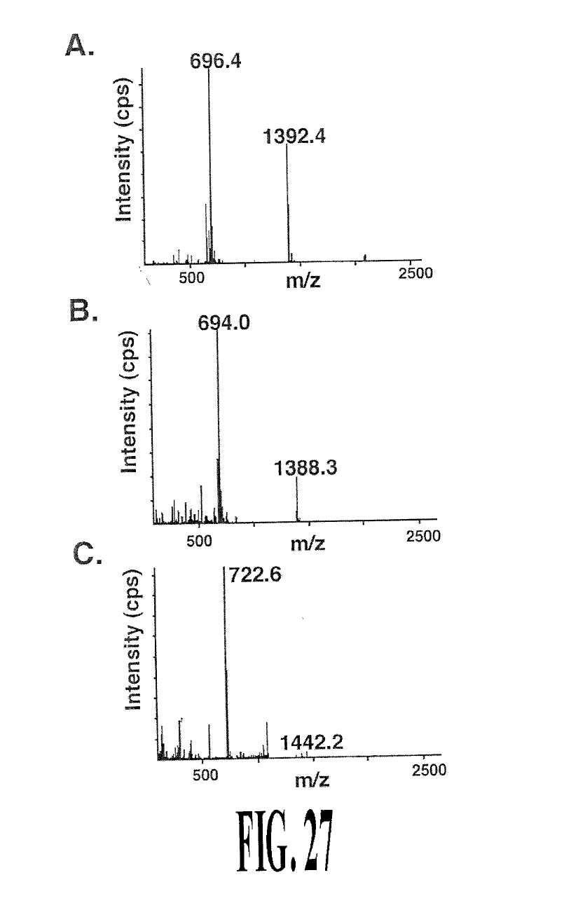

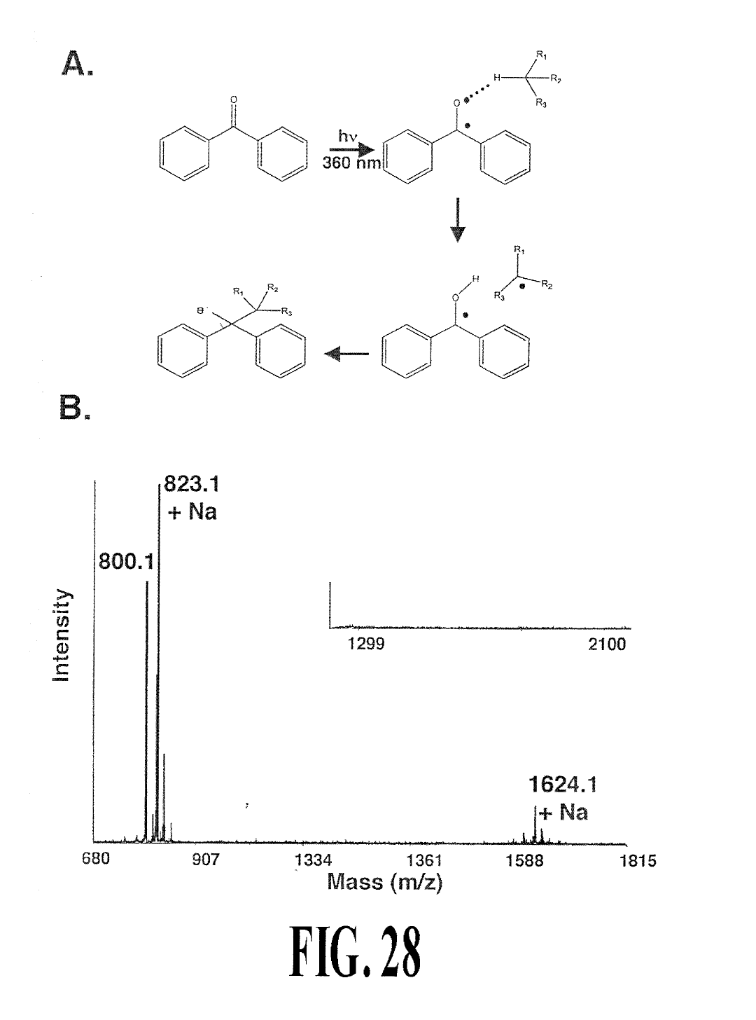

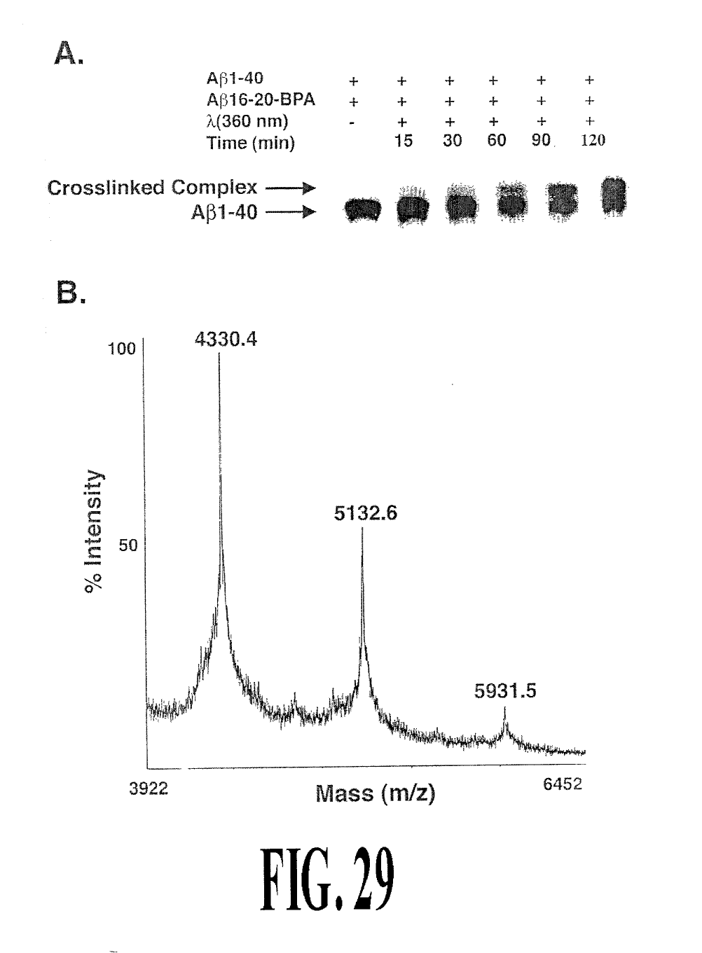

Accordingly, peptides were synthesized. Peptide I (Aβ16-22) consists of amino acids 16-22 of Aβ, and an amidated C-terminus, but contains no N-methyl amino acids. Peptides II and III (Aβ16-22m, FIG. 26, and Aβ16-22mR, respectively) contain N-methyl amino acids at alternate residues; thus these two peptides are predicted to act as inhibitors of fibrillogenesis. These two peptides differ from each other in the placement of the two charged residues, API 6-22m preserving and Aβ16-22mR reversing the positions of these two amino acids found in natural Aβ. Peptides IV and V (Aβ16-22m(4) and PrPm, respectively) also contain N-methyl amino acids, but are predicted not to act as inhibitors of fibrillogenesis. Aβ16-22m(4) has the same sequence as the previous three peptides, except that it contains N-methyl amino acids at consecutinve rather than alternate positions (FIG. 26). Consequently, if this peptide formed a β-strand, it would have N-methyl amino acids on both faces of thepeptide backbone and would be predicted to interact weakly with A(340. PrPm has N-methyl amino acids at alternate positions, but the sequence is from an unrealted protein (albeit another fibril forming one), the human prion protein. In all cases, the peptides were synthesized with amidated C-termini.

B. Synthesis of Fibrillogensis Inhibitor Peptides

Yields from syntheses of peptides containing N-methyl amino acids are not adequate if coupling reagents from standard FMOC chemistry are used. Excellent purity and yields were given by using the activating reagent HATU for the coupling steps immediately after an N-methyl amino acid.

The N-methyl amino acid containing peptides are surprisingly soluble, and solutions could be made with peptide concentrations exceeding 40 mg/ml in PBS. In contrast, the corresponding unmethylated peptides are soluble at concentrations 1-2 mg/ml, i.e. twenty to forty-fold less soluble under similar conditions.

Electron microscopy of inhibitor peptide solutions showed no fibrillar or aggregated material. This inability of Aβ16-22m to from fibrils is consistent with its high degree of solubility.

C. Inhibition and Dissasembly

Two of the N-methyl peptides, Aβ16-22 and Aβ16-22mR, prevented fibril fromation of Aβ40 in a dose dependent manner. These are the two peptides containing N-methyl amino acids in alternate positions of the sequence. FIG. 2A shows thioflavin fluorescence as a function of inhibitor concentration; since a constant concentration of Aβ40 peptide is used, this is expressed as the ration of inhibitor Aβ40 peptide. Both Aβ16-22m and Aβ16-22mR were potent inhibitors of fibrillogenesis; the IC₅₀ of Aβ16-22m and Aβ16-22mR occurred at inhibitor: Aβ40 rations of approximately 4:1 and 9:1, respectively. Incubation with greater than a 30-fold molar excess of Aβ16-22m resulted in essentially complete inhibition; for Aβ16-22mR, this occurred at higher rations, ≈50:1. The unmethylated control pepitde, Aβ16-22, had a relatively modest inhibitor effect on fibril formation. As shown in FIG. 2A, at concetrations at which Aβ16-22m inhibited fibrillogenesis completely, the unmethylated Aβ,16-22 inhibited fibrillogenesis by approximately 10-20%. Furthermore, Aβ16-22m(4), the peptide containing four consecutive N-methyl amino acids, was a less potent inhibitor of Aβ40 fibrillogenesis than either Aβ16-22m or Aβ16-22mR, the peptides with N-methylated residues in alternate positions. An unrelated, methylated peptide, PrPm, had no effect on Aβ40 fibril formation. These results were confirmed by electron microscopy, which demosntrated a complete lack of fibrils in Aβ40 samples with a 30-fold molar excess of inhibitor; EM showed round particles which may be complexes of Aβ40 and Aβ16-22m. Inhibition of fibril formation was also confirmed with a Congo Red-binding solution assay.

The inhibitor peptides, Aβ16-22m and Aβ16-22mR both were also able to dissaembly pre-formed Aβ40 fibrils. After incubation of Aβ40 for seven days to form fibrils, different concentrations of the inhibitor peptides were added to the fibril solution. The extent of disassembly was then quantitated using the thioflavin assay after three additional days of incubation at 37° C. The IC₅₀ for the disassembly occurred at inhbitor:Aβ40 ratios of approximately 10:1 and 25:1 for Aβ16-22m and Aβ16-22mR, respectively (FIG. 2B).

D. Size Exclusion Chromatography

Size exclusion chromatography with the inhibitor peptides demonstrated two peaks. The relative sizes of the two peaks was concentration dependent, with the later eluting peal predominant at lower peptide concentrations, and the earlier eluting peak became predominant. These observations are consistent with a reversibly monomer-oligomer equilibrium. The areas of the intergrated peaks from the chromatographs were used to calculate concentrations of monomer and oligomer; data were analyzed using the equation:

$${K}_{d} = \frac{\frac{n M ⇔ {A}_{n}}{{M}^{n}}}{{A}_{n}}$$

where M and Aₙ are the monomer and aggregate (oligomer) concentrations, respectively, n is the aggreagtion number, and K_{d} is the apparent dissociation constant (FIG. 4). The fit of the data to this equation is most consistent with an aggregation number of two, i.e., a monomer-dimer equilibrium.

E. Circular Dichroism

N-methyl amino acids destabilize c-helices, and tend to promote the β-sheet geometry. The CD spectra of Aβ16-22m and Aβ16-22mR, are charectistic of a β-sheet except that the minimum is shifted ot 226 nm (FIG. 5). Similar red-shifted β-sheet spectra have been observed for a number of other peptides, and this sift has been attributed to the twist of the β-sheet sheet. N-methyl groups may have electronic properties of the peptide bond, and hence, their transitions observed by CD spectroscopy. In contrast to the N-methyl peptides, the CD spectrum of the unmethylated, control peptide Aβ16-22 is that of a random coil.

The mean residue of Aβ16-22m at 226 nm (the minimum in the CD spectra) is independent of concentration (FIG. 5B). between peptide concentrations of 0.1 mg/ml and 6 mg/ml, i.e., 1% to 91% oligomer. Thus, the peptide is a β-strand even as a monomer, and the secondary structure is not induced by aggregation.

F. Protease Resistance

The unmethylated Aβ16-22 contains a predicted chymotryptic cleavage site, and was cleaved by chymotrypsin (FIGS. 6C, D). In contrast Aβ16-22m exhibited complete resistance to chymotrypsin digestion over a period of 24 hours.

### Example 20

A. Probing the Role of Backbone Hydrogen Bonding in β-Amyloid Fibrils with Inhibitor Peptides Containing Ester Bonds at Alternate Positions

The role of hydrogen bonds in fibrillogenesis through the use of peptides containing ester bonds in the place of amide bonds. To determine whether the ester substitutions would yield peptides that were effective inhibitors of fibrillogenesis, but would permit a more direct assessment of the role of hydrogen bonds in stabilizing amyloid fibrils than the incorporation of N-methyl amino acids. The latter yield peptides with an extraordinarily stable β-strand structure that completely resists denaturation by changes in pH (2-12), temperature (to 70° C.) and the addition of denaturants such as urea or guanidine HCl (to 8M). These results suggest that the N-methyl groups confer structural rigidity to the peptides. In addition, a red shift in the CD spectrum of N-methyl amino acid-containing peptides suggests that the N-methyl groups, while conferring β-strand structure, may also introduce a twist, or distortion, in the β-strand. These findings suggest that N-methyl groups may inhibit fibrillogenesis not only by interfering with hydrogen bonding, but also by introducing steric constraints that prevent the close association of β-strands. Such steric constraints could include the relative bulkiness of the N-methyl group compared to the amide proton and the twist or distortion of the β-strand caused by the N-methyl groups. Both of these factors could interfere with the efficient packing of peptides into fibrillar aggregates. These results, therefore, raise the question of the relative contributions of hydrogen bonding and steric constraints in the ability of these peptides to inhibit Aβ fibrillogenesis.

Thus, the incorporation of ester bonds constitutes a more conservative substitution for peptide bonds than the incorporation of N-methyl amino acids. In the present example, the incorporation of two ester bonds at alternate residues of the Aβ16-20 peptide, similar to the incorporation of N-methyl amino acids, results in the formation of an effective inhibitor of Aβ1-40 fibrillogenesis. The incorporation of ester groups also prevents the peptide from forming amyloid fibrils. Strikingly, analytical ultracentrifugation demonstrates that the ester peptide is predominantly monomeric, although a small amount of dimeric peptide is observed by crosslinking and ESI-MS experiments, in contrast to N-methyl amino acid-containing peptides, which cannot form dimers. The ester peptide is incorporated into stable, soluble mixed micelle-like structures with Aβ1-40, i.e., in which the Aβ1-40 does not progress to the formation of fibrils.

B. Peptide Synthesis

FIG. 1 shows the peptides synthesized for this example. The unmodified peptide, Aβ16-20 (FIG. 26A), is derived from the central, hydrophobic region of Aβ1-40 that is critical for fibrillogenesis. Although this peptide is an inhibitor of Aβ1-40, it also aggregates and forms fibrils on its own, as demonstrated herein. The ester peptide, Aβ16-20e (FIG. 26B), is identical to Aβ16-20, except that it has two amide bonds in alternating positions replaced by ester bonds. When this peptide is arrayed in an extended, β-strand conformation, the oxygen atoms of these ester bonds align on one “face” of the molecule. The N-methyl inhibitor peptide, Aβ16-20m, is displayed in FIG. 26C as a comparison to the ester peptide. This peptide is identical to Aβ16-20e except that it incorporates N-methyl groups, rather than ester groups, in alternating positions. The PrP117-121e peptide (FIG. 26D), which also contains two ester bonds, is homologous to a central region of the prion protein and was synthesized to investigate the sequence specificity of the inhibition. The final peptide, Aβ16-20-Bpa (FIG. 26E), is identical to Aβ16-20e except that Phe2O is replaced with a photoreactive benzoyl-phenylalanine (Bpa) amino acid. This peptide is used for crosslinking experiments described herein.

The ester peptides were synthesized in excellent purity and yields using established procedures. The stability of the ester linkages to hydrolysis at pH 7.4 was measured using a RP-HPLC assay. Incubation of the ester peptides in 1100 mM phosphate buffer, pH 7.4, at 37° C. for 24 h resulted in hydrolysis of 12-14% of the peptide. Incubation of the ester peptides at room temperature, however, lowered this rate of hydrolysis to 2% in 24 h. All of the experiments reported in this work, consequently, were conducted at room temperature to minimize the hydrolysis of the ester peptides.

C. Electron Microscopy

Inhibition of Aβ1-40 fibrillogenesis by the ester peptide was initially investigated with electron microscopy. The Aβ1-40 peptide was incubated with different amounts of Aβ16-20e for four days at room temperature. Aliquots were then removed from each sample and examined by electron microscopy. The solution of Aβ1-40 incubated in the absence of any inhibitor peptide exhibited long, unbranched fibrils (FIG. 8A). Some fibrillar material was also observed when Aβ1-40 was incubated with the Aβ16-20 peptide (FIG. 8B). It is not clear, though, if these fibrils are composed of Aβ1-40, Aβ16-20 or a mixture of both peptides. As demonstrated in FIG. 8C, Aβ16-20 also aggregates to form amyloid fibrils in the absence of any other peptides. Fibrillar material was not observed when Aβ1-40 was incubated with the Aβ16-20e peptide (FIG. 8D), although some amorphous material was evident. Similar results were obtained when Aβ16-20e was added to Aβ1-40 fibrils that had been pre-formed for four days before addition of the ester peptide (FIG. 8E).

D. Thioflavin T Assay

A thioflavin T assay was also used as a more quantitative assay for fibrillogenesis. FIG. 17A demonstrates that both Aβ16-20 and Aβ16-20e inhibit the fibrillogenesis of Aβ1-40 in a concentration dependent manner. The thioflavin T fluorescence is plotted as a function of the molar ratio of the inhibitor peptide to the Aβ1-40 peptide. Since a constant concentration of Aβ1-40 was used for these experiments, the molar ratio of inhibitor:Aβ1-40 represents the inhibitor concentration. The Aβ16-20e peptide is a more effective inhibitor than Aβ16-20 and its efficacy is similar to or slightly greater than that of Aβ16-20m. None of the inhibitor peptides cause any thioflavin T fluorescence when incubated alone.

The PrPe peptide does not exhibit any inhibition of Aβ1-40 fibrillogenesis. This demonstrates that the pattern of backbone hydrogen bonds alone is not sufficient to prevent fibrillogenesis, since Aβ16-20e and PrP117-121e exhibit identical backbone hydrogen bonding capabilities. Thus, side chain interactions appear to be critical for the inhibition of fibrillogenesis by A, 16-20e, as was also observed for the peptides containing N-methyl amino acids.

Aβ16-20 and Aβ16-20e are also able to disassemble pre-formed Aβ1-40 fibrils (FIG. 17B). In this experiment, Aβ1-40 was incubated in the absence of any inhibitor for four days. At this point, inhibitor peptide was added and the samples were incubated for an additional three days. Similar to the inhibition data, the disassembly of Aβ1-40 fibrils by inhibitor peptides was concentration dependent and A 16-20e was more effective than Aβ16-20. The PrP117-121 e peptide was not able to disassemble Aβ1-40 fibrils, suggesting that disassembly also requires specific sidechain interactions. Studies of Aβ16-20 revealed a subtlety in the use of thioflavin fluorescence as a technique for measuring the extent of fibril formation by this peptide, or by this peptide in the presence of Aβ1-40. Aβ16-20 does not induce thioflavin fluorescence, even under conditions in which Aβ16-20 forms typical amyloid fibrils that are readily visible by electron microscopy. In the results shown in FIGS. 17A and 17B, the addition of Aβ16-20 to Aβ1-40 leads to a loss of thioflavin fluorescence. This loss of fluorescence results either from reduction of fibrillar material, or the presence of fibrils that do not cause thioflavin fluorescence.

The inhibition and disassembly curves were fit to the equation of a hyperbola. The parameters of the hyperbola, IC₅₀ and ICₘₐₓ, are analogous to Kₘ and Vₘₐₓ of enzyme kinetics or analogous terms in hyperbolic equations for ligand-receptor interactions. The use of this equation does not imply a specific model for the inhibition by these peptides, e.g. whether the inhibitor binds Aβ1-40 in the solution or on the fibril. The equation is used to allow a more quantitative comparison of the peptides. The Aβ16-20e peptide exhibits an IC₅₀ and ICₘₐₓ for fibril inhibition of 3.7 and 100, respectively (Table 9). These values are similar to or slightly better than the IC₅₀ and ICₘₐₓ of Aβ16-20m, 6.9 and 100. In comparison, the Aβ16-20 peptide exhibits an IC₅₀ of 9.7 and an ICₘₐₓ of 100. Although it is difficult to directly compare different amyloid inhibitors, the Aβ16-20e peptide is approximately as effective as other peptide inhibitors of amyloid fibrillogenesis.

E. Congo Red Assay

Thioflavin T fluorescence is well known as a sensitive assay for the formation of amyloid fibrils. However, some peptides that form typical amyloid fibrils do not cause thioflavin fluorescence, either because the fibrils do not bind thioflavin or because binding of the dye by some proteins or peptides is not associated with fluorescence. Neither Aβ16-22 nor Aβ16-20 fibrils, for example, bind thioflavin, despite the fact that both peptides form typical amyloid fibrils visible by electron microscopy and bind Congo Red dye. For this reason, a Congo Red binding assay was also used to investigate both the formation of amyloid fibrils and the inhibition and disassembly of Aβ1-40 fibrillogenesis (FIG. 18). Congo Red, an azo dye, exhibits a characteristic increase and redshift in its absorbance spectrum when it binds to amyloid fibrils. FIG. 18A demonstrates that both fibrillar Aβ1-40 and Aβ16-20 bind Congo Red, in agreement with the results from electron microscopy. The Aβ16-20e peptide alone, however, does not cause a change in the absorbance spectrum of Congo Red, suggesting that it does not aggregate to form amyloid fibrils, again in agreement with results from electron microscopy.

FIG. 18B shows the results of a Congo Red binding assay for Aβ1-40 incubated with Ad 16-20e and for pre-formed Aβ1-40 fibrils to which Aβ16-20e was added. In both cases, the spectra for these mixtures are identical to the control spectrum of Congo Red alone. These results demonstrate that Aβ16-20e does not form fibrils by itself and both inhibits fibril formation and disassembles pre-formed Aβ1-40 fibrils.

F. Analytical Ultracentrifugation

Analytical ultracentrifugation was used to determine if the Aβ16-20e peptide forms small aggregates or oligomers. Data were collected at three rotor speeds on solutions containing three different concentrations of Aβ16-20e, 0.05 mM, 0.2 mM and 1 mM. Data are shown in FIG. 13 for the most concentrated, 1 mM, solution of Aβ16-20e. The calculated molecular weight of Aβ16-20e is 696.4. A molecular weight of 734+32 was measured in the ultracentrifugation experiment for Aβ16-20e, indicating that the peptide is predominantly or entirely monomeric.

G. Mass Spectrometry

The aggregation of Aβ16-20e was also investigated using ESI-MS, which is an established technique for studying non-covalent protein complexes. FIG. 14A is an ESI mass spectrum for a 250 μM solution of Aβ16-20e. This spectrum exhibits two major peaks at m/z 696.4 and 1391.8. Since the calculated molecular weight of monomeric Aβ16-20e is 696.4, the peak at 1391.8 demonstrates that the peptide forms a dimeric species under the conditions of ESI-MS. The ESI mass spectrometry spectrum for Aβ16-20 also exhibits a major peak at the molecular weight for a dimeric peptide (FIG. 14B). In comparison, the spectrum for the Aβ16-20m peptide exhibits at most only a very minor peak at the molecular weight for a dimeric species.

H. Bpa Crosslinking

The mass spectrometry data demonstrate that Aβ16-20e forms a dimer in solution. Since peak intensities in ESI depend on many factors and are generally not considered quantitative, we were unable to estimate the fractions of monomeric and dimeric Aβ16-20e. The analytical ultracentrifuigation results, however, suggest that Aβ16-20e is predominantly monomeric (>90%) because the measured molecular weight is close to the monomer weight and the data are best fit by a single ideal species model, as opposed to a monomer-dimer model.

In order to examine the ESI-MS data further, an analogue of Aβ16-20e, Aβ16-20-Bpa, was synthesized that contains a photoreactive L-p-benzoylphenylalanine (Bpa) amino acid (FIG. 16E). After activation at 350-360 nm, Bpa preferentially reacts with unreactive C—H bonds, even in the presence of water and other nucleophiles (FIG. 19A). Photoaffinity labeling with Bpa is highly efficient and generally exhibits excellent site specificity. FIG. 19B shows the MALDI mass spectrometry results for a 500 μM solution of Aβ16-20-Bpa that was irradiated at 350 nm for 30 minutes. Although most of the Aβ16-20e is monomeric (MW=801.1), after the irradiation a dimer (MW=1600.8) peak is also observed in the mass spectrum, which is consistent with both the ESI-MS and AUC data. In contrast to ESI-MS, a non-covalent dimer of Aβ16-20-Bpa is not observed by MALDI-MS; the inset of FIG. 19B demonstrates that crosslinking does not occur in absence of irradiation.

I. Aβ1-40 and Bpa Crosslinking

The Aβ16-20-Bpa peptide was also reacted with Aβ1-40 to determine the binding stoichiometry. FIG. 20A shows SDS-PAGE results of Aβ16-20-Bpa incubated with Aβ1-40 for various amounts of time. Irradiation of the mixture results in the formation of a complex with a molecular weight slightly greater than Aβ1-40 alone. FIG. 20B shows MALDI-MS analysis of the irradiated Aβ1-40 and Ad 16-20-Bpa mixture. Unmodified Aβ1-40 is represented by the peak at 4331.05. The peaks at 5133.24 Da and 5936.27 Da correspond to Aβ1-40 crosslinked to one and two Aβ16-20-Bpa peptides, respectively. This experiment, however, cannot address the question of whether the Aβ1-40, to which Ad 16-20e is bound, is in a monomeric or oligomeric form.

J. DPH Fluorescence

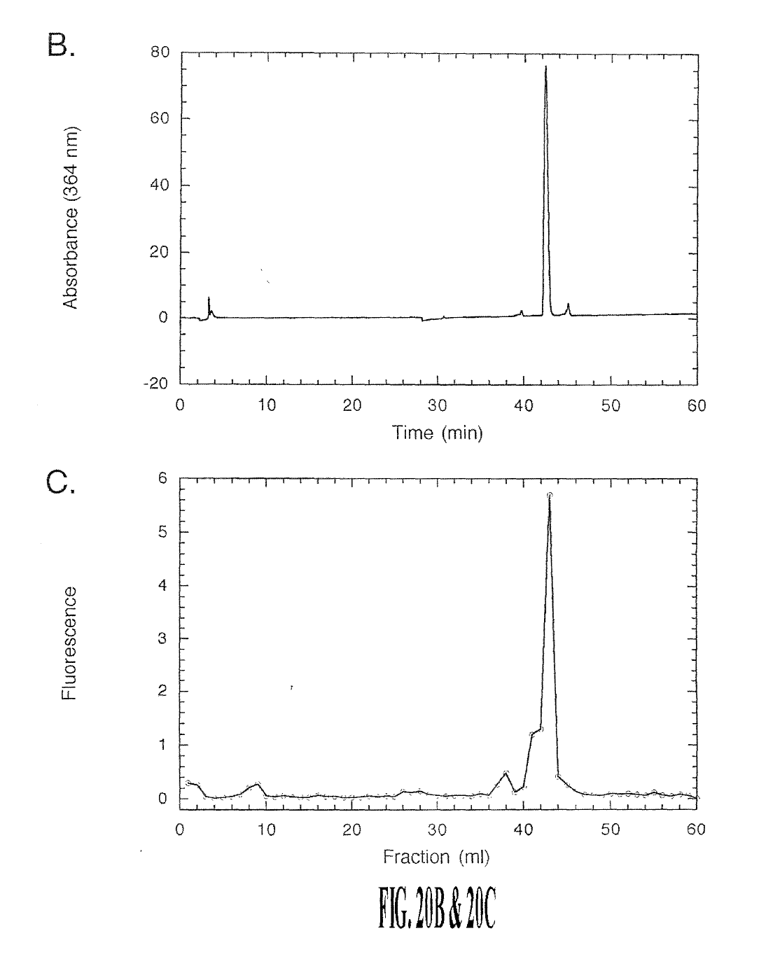

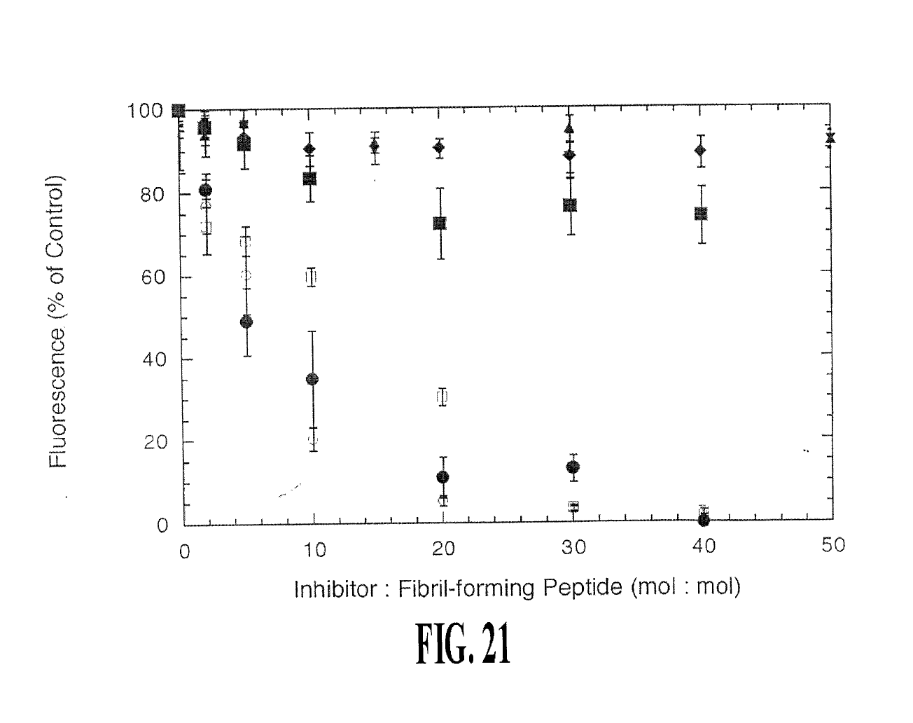

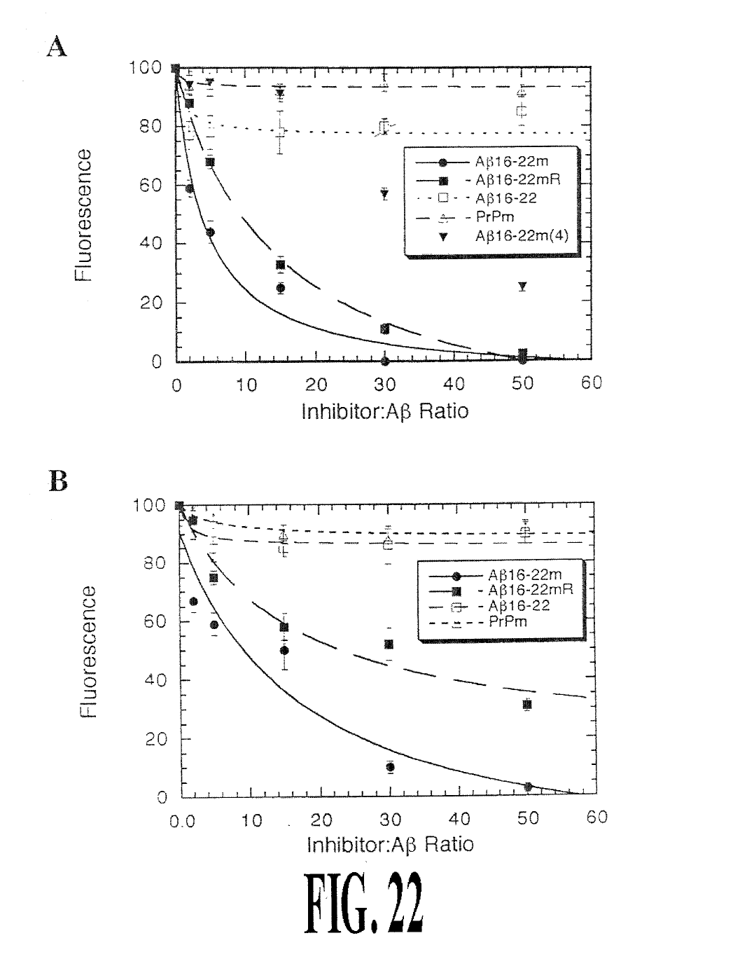

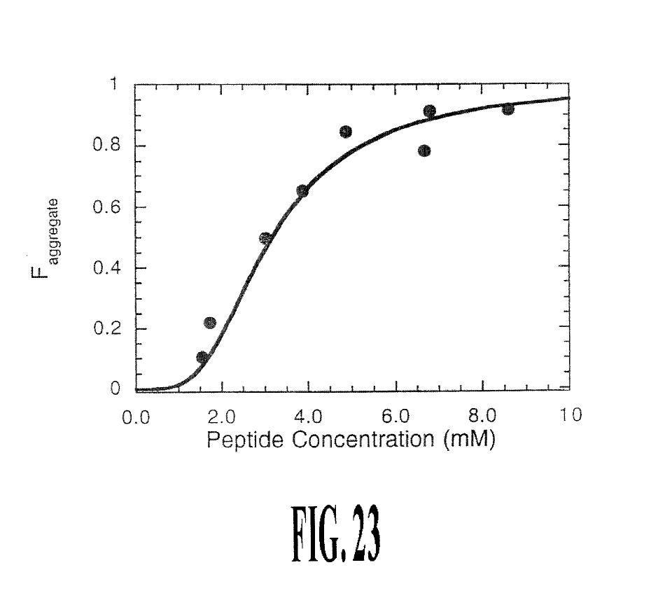

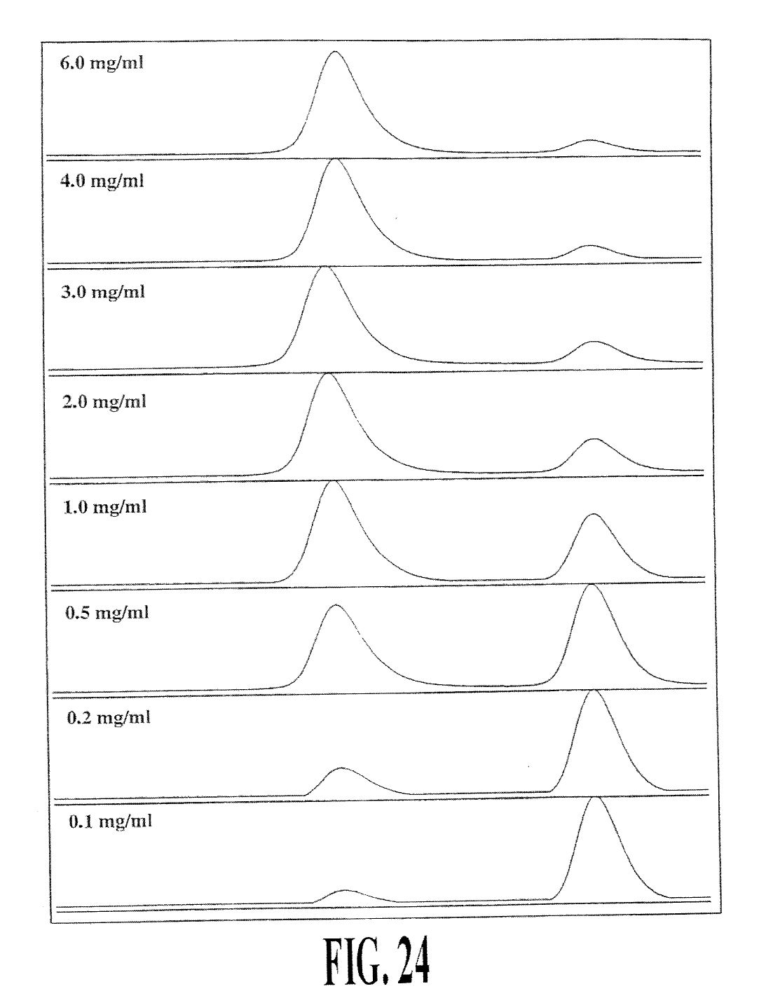

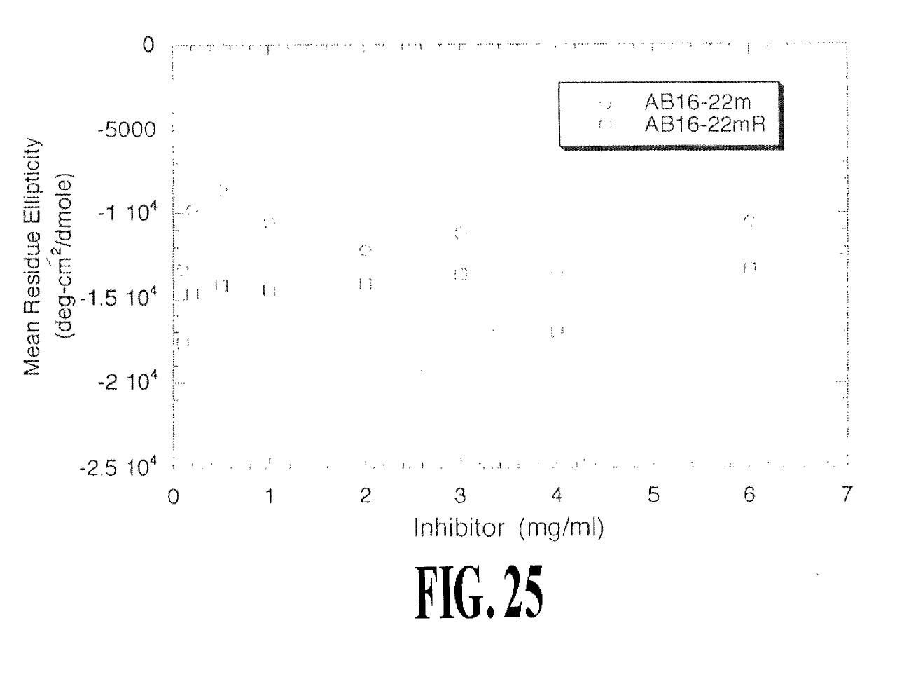

In order to investigate the state of aggregation of the Aβ1-40 peptide that was crosslinked to Aβ16-20e, we used a 1,6-diphenyl-1,3,5-hexatriene (DPH) fluorescence assay. DPH is a hydrophobic dye that exhibits a characteristic increase in fluorescence when it partitions into a hydrophobic environment. This dye was previously used to monitor the formation of a micelle-like Aβ1-40 oligomer that forms within thirty minutes of the peptide being dissolved in solution. FIG. 21A confirms data originally generated by Soreghan et al. (1994) and shows the effect of increasing Aβ1-40 concentrations on the fluorescence of DPH. Very little DPH fluorescence is observed below the critical concentration of approximately 100 μM Aβ1-40. Above this concentration, though, there is a significant increase in DPH fluorescence with increasing peptide concentration. FIG. 21B demonstrates that Aβ16-20e, even when added at a large molar excess relative to Aβ1-40, does not inhibit the formation of the micelle-like intermediate of Aβ1-40. DPH fluorescence is plotted as a function of the molar ratio of the inhibitor peptide to the Aβ1-40 peptide. In all samples, the concentration of Aβ1-40 is 150 μM and only the concentration of Aβ16-20e is varied. DPH fluorescence is not observed for either the Aβ16-20e peptide alone or monomeric Aβ1-40 in a 9M urea solution.

All of the compositions and methods disclosed and claimed herein can be made and executed without undue experimentation in light of the present disclosure. While the

compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

TABLE 7
Summary of Fibrillogenesis Inhibition and Fibril Disassembly Data.

| | Inhibition, IC₅₀ ± | Disassembly, IC₅₀ (± |
|---|---|---|
| Peptide | S.D. (R value) | S.D., R value) |
| Aβ16-20 | 9.7 ± 2.06 (0.992) | 13.5 ± 2.01 (0.993) |
| Aβ16-20m | 6.9 ± 1.95 (0.984) | 7.8 ± 1.67 (0.993) |
| Aβ16-20m2 | 5.4 ± 1.12 (0.991) | 5.5 ± 1.26 (0.989) |
| Anth-Aβ16-20m | 2.7 ± 0.277 (0.989) | 3.2 ± 0.123 (0.998) |
| Aβ16-20s | 7.7 ± 1.95 (0.988) | 7.9 ± 3.37 (0.977) |

TABLE 8
Summary of ³J_{NHα} Coupling Constants and Corresponding φ Angles.

| Residues | ³J_{HNα} | φ1 Angle | φ2 Angle |
|---|---|---|---|
| Lys1 | 7.1 | −157 | −82 |
| Val3 | 9.2 | −152 | −88 |
| Phe5 | 7.7 | −135.4 | −104.6 |

TABLE 9
Summary of fibril inhibition and disassembly data

| | |
|---|---|
| Inhibition | Disassembly |

| Peptide | IC₅₀ | ICₘₐₓ | IC₅₀ | ICₘₐₓ |
|---|---|---|---|---|
| Aβ16-20e | 3.7 | 100 | 5.2 | 100 |
| Aβ16-20m | 6.9 | 100 | 7.8 | 100 |
| Aβ16-20 | 9.7 | 100 | 13.5 | 100 |
| PrP117-121e | nd | nd | nd | nd |

TABLE 10

| | Peptide | Sequence |
|---|---|---|
| I | Aβ16-22 | NH₂-KLVFFAE-CONH₂ |
| II | Aβ16-22m | NH₂-K(me-L)V(me-F)F(me-A)-E-CONH₂ |
| III | Aβ16-22mR | NH₂-E(me-L)V(me-F)F(me-A)K-CONH₂ |
| IV | Aβ16-22m(4) | NH₂-KL(me-V)(me-F)(me-F)(me-A)-E-CONH₂ |
| V | PrPm | NH₂-GA(me-A)AAA(me-V)V-CONH₂ |

TABLE 11
Summary of Fibrillogenesis Inhibition and Fibril Disassembly Data

| | Inhibition of Fibrilogenesis | | Fibril Disassembly | |
|---|---|---|---|---|
| Peptide | IC₅₀ (μM) | ICₘₐₓ (%) | IC₅₀ (μM) | ICₘₐₓ (%) |
| Aβ16-22m | 4.2 | 100 | 6.9 | 100 |
| Aβ16-22mR | 7.8 | 100 | 23.7 | 100 |
| Aβ16-22m(4) | 38.9 | 100 | 31.6 | 100 |
| PrPm | 6.0 | 8.6 | 8.9 | 10.3 |
| Ac-Aβ16-20 | 8.4 | 100 | 11.3 | 100 |
| Aβ16-22 | 1.1 | 23.0 | 11.3 | 89.2 |
| Aβ16-20m | 0.4 | 100 | 0.9 | 100 |
| Anth-Aβ16-20 | 0.3 | 100 | 0.8 | 100 |

Materials and Methods

A. Polypeptides and Peptides

The polypeptides of the invention can also be generated by modifying the sequence of any fibril forming protein by amino-acid substitutions, replacements, insertions and other mutations to obtain fibril inhibitory and/or disassembling properties. In some cases these modification can generate polypeptides with better fibril inhibitory and/or disassembling properties. In other cases functionally equivalent polypeptides may be obtained. The following is a discussion based upon changing of the amino acids of a protein or polypeptide to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and in its underlying DNA coding sequence, and nevertheless produce a protein with like properties (see Table 11). It is thus contemplated by the inventors that various changes may be made in the polypeptide sequences of the invention with no change in the ability of the polypeptide to inhibit fibril formation or to disassemble pre-formed fibrils. In some cases substitutions of amino acids may create more potent inhibitor and disassembler polypeptides.

In making such changes, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids. can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (*−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still produce a biologically equivalent and immunologically equivalent protein. In such changes, the substitution of amino acids whose hydrophilicity values are—within +2 is preferred, those that are within +1 are particularly preferred, and those within +0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

TABLE 12

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It is envisioned that the peptides and polypeptides will include the twenty “natural” amino acids, and modifications thereof. In vitro peptide synthesis permits the use of modified and/or unusual amino acids. One of skill in the art realizes that amino acid modifications can include, but are not limited to methylation, acetylation, reduction and/or esterification of residues. Yet further, one skilled in the art also realizes that the N-terminal may be modified by a variety of compounds for example, anthranilic acid. A table (Table 12) of exemplary, but not limiting, modified and/or unusual amino acids is provided herein below.

TABLE 13
Modified and/or Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| BAla | Beta-alanine, beta-Amino- | Ahyl | allo-Hydroxylysine |
| | propionic acid | | |
| Abu | 2-Aminobutyric acid | 3Hyp | 3-Hydroxyproline |
| 4Abu | 4-Aminobutyric acid, piperidinic | 4Hyp | 4-Hydroxyproline |
| | acid | | |
| Acp | 6-Aminocaproic acid | Ide | Isodesmosine |
| Ah | 2-Aminoheptanoic acid | Aile | allo-Isoleucine |
| Aib | 2-Aminoisobutyric acid | MeGly | N-Methylglycine, |
| | | | sarcosine |
| BAib | 3-Aminoisobutyric acid | Melle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

Another embodiment for the preparation of polypeptides according to the invention is the use of peptide mimetics. Mimetics are peptide-containing molecules that mimic elements of protein secondary structure. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of the fibril inhibitor peptides of this invention, but with altered and even improved characteristics.

B. Fusion Proteins

A specialized kind of insertional variant is the fusion protein. This molecule generally has all or a substantial portion of the native molecule, linked at the—or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals or transmembrane regions. The present inventors contemplating using fusions for example to achieve targeting of cells that contain fibrils.

C. Protein Purification

It may be desirable in the context of this invention to purify fibril forming proteins or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoresis techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide: The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such, and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. “This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular 5 weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (e.g., alter pH, ionic strength, and temperature.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine. has been used in obtaining lectins from clams and L-fucose will bind to lectins from lotus.

The matrix should be a substance that itself does not adsorb molecules to any, significant extent and that has a broad range of chemical, physical and thermal stability. The ligand should be coupled in such a way as to not affect its binding properties. The ligand also should provide relatively tight binding. And it should be possible to elute the substance without destroying the sample or the ligand. One of the most common forms of affinity chromatography is immunoaffinity chromatography.

D. Antibody Production

Polyclonal antibodies to the polypeptide inhibitors of the present invention are raised in animals by multiple subcutaneous (sc) or intraperitoneal (ip) injections of the polypeptide inhibitor and an adjuvant. It may be useful to conjugate the polypeptide inhibitor to a protein that is immunogenic in the species to be immunized, e.g. keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaraldehyde, or succinic anhydride.

Animals are immunized against the immunogenic conjugates or derivatives by combining 1 mg of 1 .mu.g of conjugate (for rabbits or mice, respectively) with 3 volumes of Freud's complete adjuvant and injecting the solution intradermally at multiple sites. One month later the animals are boosted with ⅕ to 1/10 the original amount of conjugate in Freud's complete adjuvant by subcutaneous injection at multiple sites. 7 to 14 days later the animals are bled and the serum is assayed for anti-polypeptide inhibitor antibody titer. Animals are boosted until the titer plateaus. Preferably, the animal boosted with the conjugate of the same polypeptide inhibitor, but conjugated to a different protein and/or through a different cross-linking reagent. Conjugates also can be made in recombinant cell culture as protein fusions. Also, aggregating agents such as alum are used to enhance the immune response.

Monoclonal antibodies are obtained from a population of substantially homogeneous antibodies, i.e., the individual antibodies comprising the population are identical except for possible naturally-occurring mutations that may be present in minor amounts. Thus, the modifier “monoclonal” indicates the character of the antibody as not being a mixture of discrete antibodies. For example, the anti-polypeptide inhibitor monoclonal antibodies of the invention may be made using the hybridoma method first described by Kohler & Milstein, or may be made by recombinant DNA methods [Cabilly, et al., U.S. Pat. No. 4,816,567].

In the hybridoma method, a mouse or other appropriate host animal, such as hamster is immunized as hereinabove described to elicit lymphocytes that produce or are capable of producing antibodies that will specifically bind to the protein used for immunization. Alternatively, lymphocytes may be immunized in vitro. Lymphocytes then are fused with mycloma cells using a suitable fusing agent, such as polyethylene glycol, to form a hybridoma cell [Goding, Monoclonal Antibodies: Principles and Practice, pp.59-103 (Academic Press, 1986)].

E. Conjugating Peptides and/or Antibodies

A variety markers can be conjugated to antibodies or polypeptides. Examples of markers that may be used in the present invention include, but are not limited to enzymes, radiolabels, haptens, fluorescent labels, phosphorescent molecules, chemiluminescent molecules, chromophores, luminescent molecules, photoaffinity molecules, colored particles or ligands, such as biotin.

The detection of the conjugated antibody or protein may be detected by a variety of known standard procedures. Many appropriate imaging agents are known in the art, as are methods for their attachment to antibodies and polypeptides (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (HII), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

Among the fluorescent labels contemplated for use as conjugates include Alexa 350, Alexa 430, AMCA, BODIPY 630/650, BODIPY 650/665, BODIPY-FL, BODIPY—R6G, BODIPY-TMR, BODIPY-TRX, Cascade Blue; Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

In the case of radioactive isotopes for therapeutic and/or diagnostic application, one might mention astatine²¹¹ , ¹⁴carbon, ⁵¹chromium, chlorine, ⁵⁷cobalt, ⁵⁸ cobalt, copper⁶⁷, ¹⁵²Eu, gallium⁶⁷, ³hydrogen, iodine¹²³, iodine¹²⁵, iodine¹³¹ indium¹¹¹, ⁵⁹iron, ³²phosphorus, rhenium¹⁸⁶, rhenium^{188,} ⁷⁵selenium, ³⁵sulphur, technicium^{99m}

and/or yttrium⁹⁰. ¹²⁵I is often being preferred for use in certain embodiments, and technicium^{99m} and/or indium¹¹¹ are also often preferred due to their low energy and suitability for long range detection. Radioactively labeled polypeptides or antibodies of the present invention may be produced according to well-known methods in the art.

Several methods are known in the art for the attachment or conjugation of a polypeptide and/or antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in, crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

F. Synthetic Polypeptides and Peptides

The present invention describes small polypeptides and peptides synthesized based on the core sequence of various fibril forming proteins for use in various embodiments of the present invention. Such peptides should generally be at least four, or five or six amino acid residues in length, and may contain up to about 10-50 residues, however, larger polypeptides may be synthesized, for example, polypeptides comprising 100 or more residues. Because of their relatively small size, the peptides of the invention can also be synthesized in solution or on a solid support in accordance with conventional techniques.

Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, or libraries of overlapping peptides, usually from about 4 up to about 10 to β40 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression. Methods for producing peptides by recombinant DNA techniques are well known in the art.

G. Screening for Fibrillogenesis Inhibitors and Fibril Disassemblers

In certain embodiments, the present invention concerns a method for screening for candidates that are fibrillogenesis inhibitors. It is contemplated that this screening technique will prove useful in the general identification of other compounds that will inhibit, reduce, decrease or otherwise abrogate protein aggregation and fibril formation.

It is contemplated in the present invention to use known sequences of fibril forming peptides and alter these sequences to synthesize inhibitor polypeptides. Once a fibril forming peptide is identified, the inhibitor is synthesized and the inhibitors are screened using the methods described herein.

Within one example, an inhibitor screening assay is performed on a sample that has fibril forming proteins. Such a sample may comprise cells having or expressing fibril forming proteins. These cells are exposed to a candidate substance under suitable conditions, and for a time sufficient, to permit the agent to affect the formation of fibrils. The inhibition of fibrils is tested by Circular Dichroism, thioflavin T fluorescence, Congo Red binding, FTIR spectroscopy, NMR and electron microscopy (EM). The test reaction is compared to a control reaction which lacks the test sample.

A candidate inhibitor identified as a substance that decreases fibril formation. In these embodiments, the screening assay may measure some characteristic fibrils which maybe selected from the group consisting of, inhibiting fibril formation, decreasing fibril formation, inhibiting or decreasing protein aggregation, inhibiting polymerization of fibril proteins, solubilizing fibril proteins.

H. Pharmaceuticals

Aqueous compositions of the present invention comprising effective amounts of the polypeptides of the invention, may be dissolved or dispersed in a pharmaceutically acceptable carrier or medium to form diagnostic and/or therapeutic formulations of the invention.

The phrases “pharmaceutically or pharmacologically acceptable” refer to molecular entities and compositions that do not produce an adverse, allergic or other untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the 5 compositions.

The active compounds will generally be formulated for parenteral administration, e.g., formulated for injection via the intravenous, intramuscular, sub-cutaneous, intra-lesional, or even intraperitoneal routes. The preparation of an aqueous composition that contains a polypeptide will be known to those of skill in the art in light of the present disclosure. Typically, such compositions can be prepared as injectibles, either as liquid solutions or suspensions; solid forms suitable for using to prepare solutions or suspensions upon the addition of a liquid prior to injection can also be prepared; and the preparations can also be emulsified.

The pharmaceutical forms suitable for injectible use include sterile aqueous solutions or dispersions; formulations including sesame oil, peanut oil or aqueous propylene glycol; and sterile powders for the extemporaneous preparation of sterile injectible solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

Formulations of neutral or salt forms are also provided. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the-use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectible compositions can be brought about by the use in the compositions of agents delaying absorption, for example, aluminum monostearate and gelatin.

Sterile injectible solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectible solutions, the preferred methods of preparation are vacuum-drying and freeze-drying techniques which yield a powder of the active ingredient plus any additional desired ingredient from a previously sterile-filtered solution thereof.

The preparation of more, or highly, concentrated solutions for local injection also is contemplated. In this regard, the use of DMSO as solvent is preferred as this will result in extremely rapid penetration, delivering high concentrations of the active agents to a small area.

Upon formulation, solutions will be administered in a manner compatible with the dosage formulation and in such amount as is diagnostically or therapeutically effective. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In other embodiments, the administering is effected by regional delivery of the pharmaceutical composition. The administering may comprise delivering the pharmaceutical composition endoscopically, intratracheally, percutaneously, or subcutaneously. Continuous administration also may be applied where appropriate. Delivery via syringe or catherization is also contemplated.

In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated or diagnosed. The person responsible for administration will, in any event, determine the appropriate dose for the individual subject.

A typical regimen for preventing, suppressing, or treating a condition associated with fibril related pathologies, comprises either (1) administration of an effective amount in one or two doses of a high concentration of inhibitory peptides in an amount sufficient to inhibit fibril formation or dissemble preformed fibrils (2) administration of an effective amount of the peptide administered in multiple doses of lower concentrations of inhibitor peptides over a period of time up to and including several months to several years.

It is understood that the dosage administered will be dependent upon the age, sex, health, and weight of the recipient, kind of concurrent treatment, if any, frequency of treatment, and the nature of the effect desired. The total dose required for each treatment may be administered by multiple doses or in a single dose. By “effective amount”, it is meant a concentration of the inhibitor or disassembler polypeptide which is capable of inhibiting or decreasing the formation of fibrils, or of dissolving pre-formed fibril and their deposits. Such concentrations can be routinely determined by those of skill in the art. It will also be appreciated by those of skill in the art that the dosage may be dependent on the stability of the administered peptide. A less stable peptide may require administration in multiple doses.

I. Peptide Synthesis, Purification and Analysis.

The human Aβ1-40 peptide was synthesized using standard 9-fluorenylmethoxycarbonyl chemistry on an Applied Biosystems model 431A peptide synthesizer:

NH₂-DAEFRHDSGY¹⁰ EVHHQKLVFF²⁰ AEDVGSNKGA³⁰ IIGLMVGGVV⁴⁰—COOH

A fibril forming peptide (Forloni et al., 1993) derived from the human prion protein, amino acids 106-126 was synthesized with a free carboxyl terminus:

NH₂-¹⁰⁶KTNMK¹¹⁰HMAGAAAAGA¹²⁰ VVGGLG¹²⁶—COOH

Peptides with a carboxamide at the C-terminal were prepared by using FMOC-amide MBHA resin (Midwest Biotech). The N-methyl peptides were synthesized manually using 9-fluorenylmethoxycarbonyl chemistry and an amide MBHA resin (Midwest Biotech). Amino acids added after N-methyl amino acids (Novabiochem) were coupled for 3-5 hours using the HATU (PE Biosystems) activating reagent. Other residues were coupled for 1.5 hours with HBTU/HOBt (PE Biosystems). N-methyl anthranilic acid was coupled to the N-terminal of peptides using standard chemistry and coupling times. N-termini of peptides were acetylated with a 10% acetic anhydride solution in DMF. The radioactive Aβ16-20m peptide was prepared by acetylation with ¹⁴C-acetic anhydride (Amersham). The specific radioactivity of the peptide was 10,230 cpm/nmol.

The peptides were purified using a reverse-phase, C18 preparative HPLC column (Zorbax) at 60° C. Peptide purity was greater than 97% by analytical HPLC (Vydac C18 column). The molecular masses of the peptides were verified with electrospray mass spectrometry.

J. Fibrillozenesis and Fibril Disassembly Assays.

The assay used to measure the inhibitory and disassembly activity of the peptides was described in previous publications by Findeis (2000) and by Farrett et al. (1993). For an inhibition assay, the inhibitor peptide, dissolved in HFIP, was divided into aliquots. The HFIP was then evaporated under a stream of dry nitrogen. The dried peptide was redissolved in 100 mM Tris buffer, 150 mM NaCl, pH 7.4. An aliquot of Aβ1-40 peptide in HFIP was then added to the solution, containing or not containing an inhibitor peptide. The mixtures were vortexed for approximately 30 seconds and then incubated at 37° C. for 5-7 days without shaking. The final concentration of Aβ1-40 in the mixture was 100 FM. The final concentration of HFIP in the assay solutions was less than 2% (v/v), which does not inhibit fibrillogenesis.

For a disassembly experiment, Aβ1-40 was incubated alone for 5 days to allow fibrils to form. An aliquot of the formed fibrils in buffer was then added to inhibitor peptide that had been dried from HFIP. The extent of fibrils remaining intact was assayed using Thioflavin T fluorescence and electron microscopy, as described below.

Data were fit to the equation for a hyperbola:

$$\% Fluorescence = 100 \% - \frac{{IC}_{max} [ P ]}{{IC}_{50} + [ P ]}$$

where P is the hlhibitor:Aβ1-40 ratio and the two parameters, IC₅₀ and ICₘₐₓ, are analogous to parameters of equations for ligand-receptor interactions or Michaelis-Menten kinetics. Because a constant concentration of Aβ1-40 was used for these experiments, P is a measure of the inhibitor concentration.

The kinetic data were fit to the equation for a pseudofirst order rate process:

Fluorescence=(A₀−A_{final})e^{-kt}+A_{final}

where A₀ is the fluorescence in the absence of inhibitor and A_{final} is the final fluorescence value.

K. Fluorescence Spectroscopy.

Fluorescence experiments were performed as described by Naiki and Nakakuki (1996) using a Hitachi F-2000 fluorescence spectrophotometer. The Thioflavin T solution contained 5 μM Thioflavin T in 50 mM glycine-NaOH buffer, pH 8.5. A 5 μl aliquot of solution containing fibrils was added to 1 ml of the Thioflavin T solution. The solution was mixed vigorously and the signal was then averaged for 30 seconds. The excitation and emission wavelengths were 446 nm and 490 nm, respectively.

L. Vesicle Efflux.

¹⁴C-Aμ16-20m and ³H-glycine (Amersham) were dissolved in 100 mM phosphate buffer at concentrations of 5 mM and 0.5 mM, respectively. Phosphatidylcholine (Avanti Polar Lipids), dissolved in chloroform, was dried under a stream of nitrogen and then stored under vacuum overnight. The dried lipids were rehydrated with the Aβ16-20m and glycine solutions, vortexed for several minutes and subjected to five freeze/thaw cycles. The lipid suspensions were extruded through a membrane with a 100 nm pore size using a mini-extruder (Avanti Polar Lipids). The vesicles were then separated from free Aβ16-20m and glycine by passage over a PD-10 Sephadex G-25 column (Pharmacia). The vesicle solution was incubated at 37° C. during the assay.

The efflux of radioactive material from the vesicles was monitored essentially as described by Austin et al (1995, 1998). Briefly, the effluxed Aβ16-20m and glycine were separated from the vesicles by ultrafiltration through Microcon Microcentrators (Amicon) with a molecular weight cutoff of 3000. A 200 gl aliquot of the vesicle solution was spun for 20 minutes at 14000 g. The radioactivity, ¹⁴C and 3H, present in the filtrate was quantitated by scintillation counting. The total radioactivity was determined by adding 0.1% Triton X-100 to an aliquot of vesicle solution and then centrifuging. Comparison of the total radioactivity determined by this method and by sampling the vesicle solution directly, without the subsequent centrifugation step, revealed that approximately 5% of the material was retained on the filter.

M. Calcein Leakage Assay.

The leakage of vesicle contents was monitored by measuring the release of calcein (Terzi et al., 1995; Pillot et al., 1996). Vesicles were prepared and separated from free calcein as described herein for the radioactive compounds, except that the rehydration buffer contained 40 mM calcein and 1 mM Na-EDTA. Different amounts of either Aβ16-20 or Aβ16-20m were added to the vesicle solutions and the fluorescence was measured with excitation and emission wavelengths of 490 and 520 nm, respectively, after a two hour incubation at 37° C. The maximum fluorescence was measured by lysing the vesicles with the addition of 0.1% (w/v) Triton X-100.

N. Right Angle Light Scattering.

The effect of Aβ16-20m on vesicle size was monitored by following the change in 90° light scattering. Vesicles were prepared as described herein. The 90° light scattering of vesicle solutions in the presence or absence of peptide were measured on a Hitachi F-2000 spectrofluorimeter with both the excitation and emission wavelengths set to 600 nm.

O. Cell Assays.

COS cells, plated on coverslips, were incubated overnight in the presence of 4 μM to 40 μM of the Anth-Aβ16-20 peptide. The cells on coverslips were then washed extensively with PBS, fixed for one hour with a 3.7% formaldehyde solution and mounted on a slide. The cells were examined by fluorescence microscopy using a DAPI filter.

Anth-Aβ16-20m peptide that had been internalized by COS cells was also reisolated to ensure that the peptide had not been degraded or modified. In this experiment, Anth-Aβ16-20m was incubated with COS cells for eight hours. The cells were then washed extensively with media until the washes did not exhibit any fluorescence. The cells were then lysed by the addition of Triton X-100 to 0.1% (v/v), and the lysate was analyzed by HPLC. The HPLC solvent system contained 0.1% trifluoroacetic acid in water (solvent A) and 0.1% trifluoroacetic acid in acetonitrile (solvent B). The peptide was eluted with a gradient of 0%-60% solvent B in 60 minutes. Fractions (1 ml) from the HPLC were collected and analyzed by fluorescence spectroscopy. The excitation and emission wavelengths were 346 nm and 435 nm, respectively.

P. Analytical Ultracentrifugation.

Sedimentation equilibrium experiments were performed using a Beckman Optima XLA ultracentrifuge equipped with an An60Ti rotor and analytical cells with six-channel centerpieces. Aβ16-20m was dissolved in 100 mM phosphate buffer, pH 7.4, 150 mM NaCl at a concentration of 1 mM. The equilibrium distribution of peptide was measured at 20° C. with a rotor speeds of 36,000, 42,000 and 48,000 rpm. Scans were performed by measuring the TV absorbance at 256 nm. Fifty scans were averaged at each point with a step size of 0.001 cm. Duplicate scans taken 4 hours apart were overlaid to determine whether equilibrium had been attained. Partial specific volumes were estimated from amino acid composition and solvent density was calculated using the SEDNTERP program.

Q. Electron Microscopy.

After incubation of the inhibition and disassembly samples for the appropriate period of time, an aliquot of each sample was applied to a glow-discharge, 400-mesh, carbon-coated support film and stained with 1% uranyl acetate. Micrographs were recorded using Philips EM300 at magnifications of 17,000, 45,000 and 100,000.

R. Circular Dichroism.

The circular dichroic (CD) spectra were recorded using a Jasco P715 spectropolarimeter. For the concentration dependency experiment, Aβ16-20m, at concentrations ranging from 0.01 mM to 11 mM, was dissolved in 100 mM phosphate buffer at pH 7.4. A 1 mm or 0.1 mm pathlength cell was used for measurements, depending on the concentration of the solution. Six to eight scans were acquired from 250 nm to 200 nm. For the pH experiment, a 100 mM phosphate-citrate buffer was used for pH 2.5-6.5, a 100 mM phosphate buffer was used for pH 7.5-8.5 and a 100 mM glycine-NaOH buffer was used for pH 9.5-10.5. For the urea denaturation experiment, Ad 16-20m was dissolved in 100 mM phosphate buffer pH 7.4 with 0-8.5 M urea.

S. Nuclear Magnetic Resonance.

The NMR data collection is described by Benzinger et al. (1998). Briefly, NMR samples were prepared by dissolving the Aβ16-20m peptide in a solution of 100 mM phosphate buffer at pH 4.5 with 10% D₂O (v/v). The 1D spectra were recorded on a 1 mM Aβ16-20m sample. The 2D spectra were collected on a 30 mM Aβ16-20m sample. The NMR experiments were performed on a Varian 600 MHz spectrometer at 15° C. Typical two dimensional data were recorded with 256 free induction decays (FIDs) of 2k data points, 16 scans per FID and a spectral width of 6000 Hz in both dimensions. Presaturation was used for water suppression, which included 2.5 s of continuous irradiation. The ROESY and TOCSY spectra were recorded with mixing times of 300 ms and 50 ms, respectively. All samples were referenced to DSS (0 ppm) as the internal standard. Data were processed using the Varian VNMR version 6.1b software. The φ torsional angles were estimated from the equation from Wüthrich (32), i.e., ³J_{HNα}=6.4 cos²θ−1.4 cos θ+1.9, where θ=|φ−60|

T. Peptide Synthesis, Purification and Analysis

The human Aβ40 peptide was synthesized using standard FMOC chemistry on an Applied Biosystems model 431A peptide synthesizer. The N-methyl peptides were synthesized manually using FMOC chemistry and an MBHA amide resing (Midwest Biotech). Amino acids added after N-methyl amino acids (Novabiochem) were coupled for 3-5 hours using the HATU (PE Biosytems) activating reagent. The petides were purified to >95% using C18 preparative HPLC column (Rainin Dynamax) at 60° C. The molecular masses and purity of the peptides were erified with electrospray mass spectrometry and analytical HPLC.

U. Size Exclusion Chromatography

Size exclusion chromatography was performed using Superdex 75 (Pharmacia), Superdex Peptide HR10/30 (Pharmacia) and Shodex KW-802.5 columns (Thomson Instruments); both column and peptide samples were equilibrated with 100 mM phosphate buffer, 150 mM NaCl, pH 7.4 (PBS).

V. Chymotrypsin Digestion

The peptides were dissolved in 0.5% ammonium bicarbonate at a concentration of 1.0 mg/ml. Chymotrypsin (Worthington Biochemical Corporation) was added to a final concentration was 0.1 mg/ml. Samples were incubated at 37° C. After twenty-four hours, the samples were lyophilized and then analyzed by reverse-phase HPLC (Rainin-Microsorb C18 column) and a water-acetonitrile (0.1% (v/v) TFA) gradient (10-70% acetonitrile over one h).

W. Congo Red Binding

The Congo Red binding assay was performed essentially as described in other publications (Klunk 1989). An aliquot of peptide solution containing 50 μg of peptide was added to 1 ml of a 3 pM solution of Congo Red in 100 mM phosphate buffer, pH 7.4. The solution was incubated for 15 min at room temperature and then the absorbance was measured from 400-600 nm.

X. Electron Microscopy.

For the electron microscopy, aliquots of the inhibition and disassembly samples were applied to a glow-discharge, 400-mesh, carbon-coated support film and stained with 1% uranyl acetate. Micrographs were recorded using a Philips EM300 at magnifications of 17,000, 45,000 and 100,000.

Y. Analytical Ultracentrifugation.

Equilibrium analytical ultracentrifugation experiments were performed using a Beckman Optima XLA ultracentrifuge equipped with an An60Ti rotor and analytical cells with six-channel centerpieces. A 16-20e was dissolved in 100 mM phosphate buffer, pH 7.4, 150 mM NaCl at concentrations of 0.05 mM, 0.2 mM and 1 mM. The equilibrium distribution of peptide was measured at 20° C. with rotor speeds of 36,000, 42,000 and 48,000 rpm. Scans were performed by measuring the UV absorbance at 220 nm and 256 nm. Twenty scans were averaged at each point with a step size of 0.001 cm. Scans taken 4 hours apart were overlaid to determine whether equilibrium had been attained. Partial specific volumes were estimated from amino acid composition and solvent density was calculated using the SEDNTERP program.

Z. Photoaffinity Crosslinking.

Aβ16-20-Bpa (500 μM) was incubated either alone or in the presence of Aβ1-40 (100 μM) for 30 min at room temperature. The mixture was then irradiated at 350 nm in a Hitachi F-2000 fluorescence spectrophotometer for 90 min at room temperature. During the irradiation, aliquots of the mixture were removed at several points and analyzed by SDS-PAGE or MALDI-MS.

AA. SDS-PAGE Analysis.

Tris-Tricine SDS-PAGE was performed as described by Schagger and von Jagow (1987). Coomassie Blue staining was used to detect the peptide bands.

BB. Mass Spectrometry.

Matrix-assisted laser desorption ionization-time of flight (MALDI-TOF) mass spectrometry was performed using a Perseptive Biosystems Voyager DE Pro (Framingham, Mass.) instrument in the positive ion mode. The samples were prepared by mixing peptide solutions with an equal volume of a-cyano-4-hydroxycinnamic acid (saturated solution in 50% acetonitrile/0.1% TFA) matrix solution. Approximately 1 μl of the mixture was placed on the sample holder and allowed to dry at room temperature. Spectra of peptides were then acquired in either the linear or reflected mode with an accelerating voltage of 20-25 kV. Each spectrum was produced by accumulating data from 100-200 laser shots.

Electrospray ionization mass spectrometry (ESI-MS) was performed using a Perkin-Ehner-Sciex API-300 instrument in the positive ion mode. The peptides were prepared in either deionized water or 5 mM NH₄HCO₃ and infused into the MS at a flow rate of 5 μl/min using a syringe pump. Experiments were performed with a capillary voltage of 5 kV, orifice voltage of 30 V and a ring voltage of 300 V. Spectra were analyzed using the Biomultiview program provided by the manufacturer (Perkin-Elmer).

CC. DPH Fluorescence.

Fluorescence measurements were performed using a Hitachi F-2000 fluorescence spectrophotometer. Samples were prepared as described above for the fibrillogenesis inhibition assay, except that the buffer contained 5 μM 1,6-diphenyl-1,3,5-hexatriene (DPH, Molecular Probes). The fluorescence measurements were taken after incubating the samples for 30 minutes in the dark. The excitation and emission wavelengths were 358 nm and 430 nm, respectively.

### DOCUMENTS CITED

The following documents, to the extent that they provide exemplary procedural or other details supplementary to those set forth herein, are incorporated herein by reference.

Austin, R. P., Burton, P., Davis, A. M., Manners, C. N. and Stansfield, M. C. (1998). The effect of ionic strength on liposome-buffer and 1-octanol-buffer distribution coefficients. J. Pharm. Sci. 87, 599-607.

Austin, R. P., Davis, A. M. and Manners, C. N. (1995). Partitioning of ionizing molecules between aqueous buffers and phospholipid vesicles. J. Pharm. Sci. 84, 1180-1183.

Antzutkin O N, Balbach J J, Leapman R D, Rizzo N W, Reed J, Tycko R. Multiple quantum solid-state NMR indicates a parallel, not antiparallel, organization of β-sheets in Alzheimer's β-amyloid fibrils. Proc. Natl. Acad. Sci. USA. 97, 13045-50 (2000).

Arnett E M, Mitchell E J, Murty T S S R. Comparison of hydrogen-bonding and proton-transfer to some Lewis-bases. J. Am. Chem. Soc. 96, 3875-3891 (1974).

Baca M, Kent S B H. Direct Observation of a Ternary Complex Between the Dimeric Enzyme HIV-1 Protease and a Substrate-Based inhibitor. J. Am. Chem. Soc. 114, 3992-3993 (1992).

Balbach, J. J., Ishii, Y., Antzutkin, O. N., Leapman, R. D., Rizzo, N. W., Dyda, F., Reed, J., and Tycko, R. (2000) Amyloid fibril formation by Aβ16-22, a seven-residue fragment of the Alzheimer's β-amyloid peptide, and structural characterization by solid state NMR. Biochemistry. 39,13748-13759.

Beligere G S, Dawson P E. Design, Synthesis and Characterization of 4-Ester CI2, a Model for Backbone Hydrogen Bonding in Protein a-helices. J. Am. Chem. Soc. 122, 12079-12082 (2000).

Benzinger, Gregory, Burkoth, Miller-Auer, Lynn, Botto, Meredith, (1998). Propagating structure of Alzheimer's β-amlyoid(10-35) is parallel β-sheet with residues in exact register. Proc. Natl. Acad. Sci. USA, 95:13407-13412.

Benzinger, Gregory, Burkoth, Miller-Auer, Lynn, Botto, Meredith, (2000). Two-dimensional structure of β-amyloid(10-35) fibrils. Biochemistry, 39:3491-3499.

Benzinger, T. L. S., Braddock, D. T., Dominguez, S. R., Burkoth, T. S., Miller-Auer, H., Subramanian, R. M., Fless, G. M., Jones, D. N., Lynn, D. G., and Meredith, S. C. (1998). Structure-function relationships in side chain lactam cross-linked peptide models of a conserved N-terminal domain of apolipoprotein. Biochemistry 37, 13222-13229.

Bitan G, Lomakin A, Teplow D B. Amyloid beta-protein oligomerization: prenucleation interactions revealed by photo-induced cross-linking of unmodified proteins. J. Biol. Chem. 276, 35176-84 (2001).

Bramson H N, Thomas N E, Kaiser E T. The use of N-methylated peptides and depsipeptides to probe the binding of heptapeptide substrates to cAMP-dependent protein kinase. J. Biol. Chem. 260, 15452-7 (1985).

Burdick, D., Soreghan, B., Kwon, M., Kosmoski, J., Knauer, M., Henschen, A., Yates, J., COtman, C., Glabe, C. (1992). Assembly and aggregation properties of synthetic Alzheimer's A4/β amyloid peptide analogs. J. Biol. Chem. 267, 546-544.

Burkoth, Benzinger, Jones, Hallenga, Meredith, Lynn, JAm. Chem. Soc., 120:7655-7656, 1998.

Burton, Conradi, Ho, Hilgers, Borchardt, JPharmaceutical Sci., 85:1336-1340, 1996.

Carpino, EI-Faharn, Minor, Alberico, F. J. (1994). Advantageous applications of azabenzotriazole (triazolopyridine)-based coupling reagents to solid-phase peptide synthesis. J. Chem. Soc. Chem. Commun., 2:201-203.

Carpino, L. A. (1993). 1-hydroxy-7-azabenzotriazole—an efficient peptide coupling additive. J. Am. Chem. Soc., 115:4397-4398.

Castano, E. M., Ghiso, J., Prelli, F., Gorevic, P. D., Migheli, A., Frangione, B. (1986). In vitro formation of amyloid fibrils from two synthetic peptides of different lengths homologous to Alzheimer's disease O-protein. Biochem. Bophys. Res. Commun. 141: 782-789.

Cataldo A. M., Barnett J. L., Pieroni C., Nixon R. A. (1097) J. Neurosci. 17, 6142-51.

Chapman E, Thorson J S, Schultz P G. Mutational Analysis of Backbone Hydrogen Bonds in Staphylococcal Nuclease. J. Am. Chem. Soc. 119, 7151-7152 (1997).

Chen X G, Brining S K, Nguyen V Q, Yergey A L. Simultaneous assessment of conformation and aggregation of beta-amyloid peptide using electrospray ionization mass spectrometry. FASEB J. 11, 817-23 (1997).

Chitnumsub, Fiori, Lashuel, Diaz, and Kelly (1999). The nucleation of monomeric parallel β-sheet-like structures and their self-assembly in aqueous solution. Bioor. Med. Chem. 7, 39-59.

Clark, Buriak, Kobayashi, Isler, McRee, Ghadiri (1998). Cylindrical β-sheet peptide assemblies. J Am. Chem. Soc., 120:8949-8962.

Cody, He, Reily, Haleen, Walker, Reyner, Stewart, and Doherty, J. Med. Chem. 40: 2228-2240, 1997.

Cole, J L, Hansen J C. Analytical ultracentrifugation as a contemporary biomolecular research tool. J Biomol. Tech. 10, 163-176 (1999).

Coombs G S, Rao M S, Olson A J, Dawson P E, Madison E L. Revisiting catalysis by chymotrypsin family serine proteases using peptide substrates and inhibitors with unnatural main chains. J. Biol. Chem. 274, 24074-9 (1999).

Coste, Dufour, Pantaloni, Castro, (1990). BROP-A new reagent for coupling N-methylated amino-acids. Tetrahedron Letters 31:669-672, 1990.

Coste, Frerot, Jouin, P., and Castro B. (1991). Oxybenzotriazole free peptide coupling reagents for N-methylated amino-acids. Tetrahedron Lett., 32:1967-1970, 1991.

DeGrado, Musso, Lieber, Kaiser, Kezdy, Biophys. J, 37:329-338, 1982.

Dive, Yiotakis, Rournestand, Gilquin, Labadie, and Toma, Int. J. Peptide Protein Res. 39, 506-515, 1992.

Doig, J (1997). A three stranded betao-sheet peptide in aqueous solution containing N-methyl amino acids to prevent aggregation. Chem. Soc., Chem. Commun., 22:2153-2154.

Dorman G, Prestwich G D. Benzophenone photophores in biochemistry. Biochemistry. 33, 5661-73 (1994).

Dragovich, Webber, Prins, Zhou, Marakovits, Tikhe, Fuhrman, Patick, Matthews, Ford (1999). Bioorg. Med. Chem. Letts., 9:2189-2194.

Egnaczyk G F, Greis K D, Stimson E R, Maggio J E. Photoaffinity cross-linking of Alzheimer's disease amyloid fibrils reveals interstrand contact regions between assembled beta-amyloid peptide subunits. Biochemistry. 40, 11706-14 (2001).

Findeis M A, Musso G M, Arico-Muendel C C, Benjamin H W, Hundal A M, Lee J, Chin J, Kelley M, Wakefield J, Hayward N J. Molineaux S M. Modified-peptide inhibitors of amyloid P-peptide polymerization. Biochemistry 38, 6791-6800 (1999).

Findeis, M. A. (2000). Approaches to discovery and characterization of inhibitors of amyloid β-peptide polymerization. Biochem Biophys Acta. 1502, 76-84.

Forloni, G., Angeretti, N., Chiesa, R., Monzani, E., Salmona, M., Bugiani, O., and Taglivini, F. (1993). Neurotoxicity of a prion protein fragment. Nature: 362:543-546.

Games, Adams, Alessandrini, Barbour, Berthelette, Blackwell, Carr, Clemens, Donaldson, Gillespie et al., Nature, 373:523-527, 1995.

Geula, C., Wu, C., Saroff, L., Yuan, M. and Yankner, B. (1998) Nat. Med. 4, 827-831. Geula, Wu, Saroff, Yuan, Yankner, Nat. Med., 4:827-831, 1998.

Ghadiri, Kobayashi, Granja, Chadha, and McRee, Agnew. Chem. Int. Ed. Engl. 34, 93-95, 1995.

Ghanta, Shen, Kiessling, Murphy, JBio. Chem., 271:29525-29528, 1996.

Glenner and Wong, Biochem. Biophys. Res. Commun., 120:885-890,1984. 15

Gregory, D. M., Benzinger, T. L., Burkoth, T. S., Miller-Auer, H., Lynn, D. G., Meredith, S. C., and Botto, R. E. (1998) Dipolar recoupling NMR of biomolecular self-assemblies: determining inter- and intrastrand distances in fibrilized Alzheimer's -amyloid peptide. Solid State Nucl. Magn. Reson. 13, 149-166.

Hardy, Trends Neurosci., 20:154-159, 1997.

Harper J D, Lansbury P T Jr. Models of amyloid seeding in Alzheimer's disease and scrapie: mechanistic truthsand physiological consequences of the time-dependent solubility of amyloid proteins. Annu. Rev. Biochem. 66, 385-407 (1997).

Haviv, Fitzpatrick, Swenson, Nichols, Mort, Bush, Diaz, Bammert., Nguyen, Rhutasel, Nellans, Hoffman, Johnson, Greer, J. Med. Chem., 36:363-369, 1993.

Hilbich, C., Kisters-Woike, B., Reed, J., Masters, C. L., Beyreuther, K. (1991). Aggregation and secondary structure of synthetic amyloid β A4 peptides of Alzheimer's disease. J. Mol. Biol. 218: 149-163.

Hilbich, Kisters-Woike, Reed, Masters, Beyreuther K. (1992). Substitutions of hydrophobic amino acids reduce the amyloidogenicity of Alzheimer's disease β A4 peptides. J. Mol. Biol., 228:460-473.

Hsieh Y L, Cai J Y, Li Y T, Henion J D, Ganem B. Detection of Noncovalent FKBP FK506 and FKBP Rapamycin Complexes by Capillary Electrophoresis Mass-Spectrometry and Capillary Electrophoresis Tandem Mass-Spectrometry. J. Am. Soc. Mass. Spec. 6, 85-90 (1995).

Hughes, Burke, and Doig J. (2000). Inhibition of toxicity of the β-amyloid peptide fragment β-(25-35) using N-methylated derivatives: a general strategy to prevent amyloid formation. Biol. Chem. 275: 25109-25115.

Ingwall R T, Goodman M. Polydepsipeptides. III. Theoretical Conformational Analysis of Randomly Coiling and Ordered Depsipeptide Chains. Macromolecules. 7, 598-605 (1974).

Inouye H, Fraser P E, Kirschner D A. Structure of p-crystallite assemblies formed by Alzheimer β-amyloid protein analogues: analysis by x-ray diffraction. Biophys. J. 64, 502-19 (1993).

Jarrett, J. T., Berger, E. P., and Lansbury, P. T. (1993). The carbozy terminus of the β-amyloid protein is critical for the seeding of amyloid formation: implications for the pathogenesis of Alzheimer's disease. Biochemistry 32: 4693-4697.

Jureus, Lindgren, Langel, Bartfai Neuropeptides 32, 453-60, 1998. 25

Kang, J., Lemaire, H. G., Unterbeck, A., Salbaum, J. M., Masters, C. L., Grzeschik, K. H., Muthaup, G., Beyreuther, K., Muller-Hill, B. (1987). The precursor or Alzheimer's disease amyloid A4 protein resembles a cell-surface receptor. Nature 325: 733-736.

Kapurniotu A, Schmauder A, Tenidis K. Structure-Based Design and Study of Non-amyloidogenic, Double N-Methylated IAPP Amyloid Core Sequences as Inhibitors of IAPP Amyloid Formation and Cytotoxicity. J. Mol. Biol. 315, 339-50 (2002).

Kelly J W. Mechanisms of amyloidogenesis. Nat. Struct. Biol. 7, 824-6 (2000).

Kheterpal 1, Zhou S, Cook K D, Wetzel R. Aβ amyloid fibrils possess a core structure highly resistant to hydrogen exchange. Proc. Natl. Acad. Sci. USA. 97, 13597-601 (2000).

Kirschner, D. A., Inouye, H., Duffy, L. K., Sinclair, A., Kind, M., Selkoe, D. J. (1987), Synthetic peptide homologous to β protein from Alzheimer disease forms amyloid-like fibrils in vitro. Proc. Natl. Acad. Sci. USA 84:6953-6957.

Klunk W E, Pettegrew J W, Abraham D J. Quantitative evaluation of Congo Red binding to amyloid-like proteins with a β-pleated sheet conformation. J. Histochem. Cytochem. 37, 1273-81 (1989).

Koh J T, Cornish V W, Schultz P G. An experimental approach to evaluating the role of backbone interactions in proteins using unnatural amino acid mutagenesis. Biochemistry. 36, 11314-22 (1997).

Kohler & Milstein, Nature 256:495 (1975).

Koo E H, Lansbury P T Jr, Kelly J W. Amyloid diseases: abnormal protein aggregation in neurodegeneration. Proc. Natl. Acad. Sci. USA. 96, 9989-90 (1999).

Kumar, N. G., Izumiya, N., Miyoshi, M., Sugano, H. and Urry, D. W. (1975). Conformational and spectral analysis of the polypeptide antibiotic N-methylleucine gramicidin S dihydrochloride by nuclear magnetic resonance. Biochemistry 14, 2197-2207.

LaFerla, Tinkle, Breberich, Haudenschild, Jay, Nat. Genetics, 9:21-29, 1995.

Lambert, Barlow, Chromy, Edwards, Freed, Liosatos, Morgan, Rozovsky, Trommer, Viola et al., Proc. Natl. Acad. Sci. USA 95:6448-6453, 1998.

Lansbury, Jr., Neuron, 19:1151-1154, 1997. Levine, eurobiology Aging 16, 755-764, 1995

Lansbury P T, Costa P R, Griffiths J M, Simon E J, Auger M, Halverson K J, Kocisko D A, Hendsch Z S, Ashburn T T, Spencer R G. Structural model for the β-amyloid fibril based on interstrand alignment of an antiparallelβ-sheet comprising a C-terminal peptide. Nat. Struct. Biol. 2, 990-8 (1995).

Levine H 3rd. Soluble multimeric Alzheimer p(1-40) pre-amyloid complexes in dilute solution. Neurobiol Aging. 16, 755-64 (1995).

Li Y T, Hsieh Y L, Henion J D, Senko M W, McLafferty F W, Ganem B. Mass-Spectrometric Studies on Noncovalent Dimers of Leucine-Zipper Peptides. J. Am. Chem. Soc. 115, 8409-8413 (1993).

Lomakin A, Chung D S, Benedek G B, Kirschner D A, Teplow D B. On the nucleation and growth of amyloid beta-protein fibrils: detection of nuclei and quantitation of rate constants. Proc. Natl. Acad. Sci. USA. 93, 1125-9 (1996).

Lomakin A, Teplow D B, Kirschner D A, Benedek G B. Kinetic theory of fibrillogenesis of amyloid beta-protein. Proc. Natl. Acad. Sci. USA. 94, 7942-7 (1997).

Lorenz S A, Maziarz E P, Wood, T D. Using solution phase hydrogen/deuterium (H/D) exchange to determine the origin of non-covalent complexes observed by electrospray ionization mass spectrometry: in solution or in vacuo? J. Am. Soc. Mass. Spectrom. 12, 795-804 (2001).

Lu W Y, Starovasnik M A, Dwyer J J, Kossiakoff A A, Kent S B, Lu W. Deciphering the role of the electrostatic interactions involving Gly7O in eglin C by total chemical protein synthesis. Biochemistry. 39, 3575-84 (2000).

Lu, Morrow, and Weisgraber, J. Biol. Chem. 275, 20775-20781, 2000.

Lu W, Randal M, Kossiakoff A, Kent S B. Probing intermolecular backbone H-bonding in serine proteinase-protein inhibitor complexes. Chem. Biol. 6, 419-27 (1999).

Maggio J E, Stimson E R, Ghilardi J R, Allen C J, Dahl C E, Whitcomb D C, Vigna S R, Vinters H V, Labenski M E, Mantyh P W. Reversible in vitro growth of Alzheimer disease beta-amyloid plaques by deposition of labeled amyloid peptide. Proc. Natl. Acad. Sci. USA. 89, 5462-6 (1992).

Manavalan and Momany, Biopolymers, 19:1943-1973, 1980.

Meyer, J. -P., Davis, P., Lee, K. B., Porreca, F., Yamamura, H. I., and Hruby, V. J. (1995) Synthesis using a Fmoc-based strategy and biological activities of some reduced peptide bond pseudopeptide analogues of dynorphin A1. J. Med. Chem. 38, 3462-3468.

Nalki, H., and Nakakuki, K. (1996). First-order kinetic model of Alzheimer's β-amyloid fibril extension in vitro. Lab. Invest. 74:374-383.

Naiki H, Higuchi K, Hosokawa M, Takeda T. Fluorometric determination of amyloid fibrils in vitro using the fluorescent dye, thioflavin T1. Anal. Biochem. 177, 244-9 (1989).

Nesloney, C. L., and Kelly, J. W. (1996) A 2,3′-substituted biphenyl-based amino acid facilitates the formation of a monomeric β-hairpin-like structure in aqueous solution at elevated temperature. J. Am. Chem. Soc. 118, 5836-5845.

Osterman and Kaiser, J. Cell. Biochem., 29:57-72, 1985.

Pallitto, Ghanta, Heinzelman, Kiessling, Murphy, Biochemistry, 38:3570-3578, 1999.

Pappolla, Bozner, Soto, Shao, Robakis, Zagorski, Frangione, Ghiso, J. Biol. Chem., 10273:7185-7188, 1998.

Patel, D. J. and Tonelli, A. E. (1976). N-methyl;eucine gramicidin-S and (di-N-methylleucine) gramicidin-S conformations with cis L-Om-L-N-MeLeu peptide bonds. Biopolymers 15, 1623-1635.

Phillips S T, Rezac M, Abel U, Kossenjans M, Bartlett Pa. “@-Tides”: The 1,2-Dihydro-3(6H)-pyridinone Unit as a beta-Strand Mimic. J. Am. Chem. Soc. 124, 58-66 (2002).

Pike, Walencewicz, Glabe, Cotman, Brain Res., 563:311-314,1991.

Pillot, T., Goethals, M., Vanloo, B., Talussot, C., Brasseur, R., Vandekerckhove, J., Rosseneu, M. and Lins, L. J. (1996). Fusogenic properties of the C-terminal domain of the Alzheimer β-amyloid peptide. J. Biol. Chem. 271: 28757-28765.

Pluta, Barcikowska, Januszewski, Misicka, Lipkowski, Neuroreport, 7:1261-1265, 1996. Poduslo, Curran, Kumar, Frangione, Soto, J. Neurobiol., 39:371-382, 1999.

Poduslo, Curran, Sanyal, Selkoe; Neurobiol. Dis., 6:190-199, 1999.

Pramanik B N, Bartner P L, Mirza U A, Liu Y H, Ganguly A K. Electrospray ionization mass spectrometry for the study of non-covalent complexes: an emerging technology. J. Mass. Spectrom. 33, 911-20 (1998).

Prestwich G D, Dorman G, Elliott J T, Marecak D M, Chaudhary A. Benzophenone photoprobes for phosphoinositides, peptides and drugs. Photochem. Photobiol. 65, 222-34 (1997).

Rajarathnam, Sykes, Kay, Dewald, Geiser, Baggiolini, Clark-Lewis (1994). Neutrophil activation by monomeric interleukin-8. Science, 264:90-92.

Rochet and Lansbury, Jr. Curr. Opin. Structural Biol., 10:60-68, 2000.

Ramakrishnan C, Mitra J. Dimensions of the ester unit. Proc. Indian Acad. Sci., Sect. A. 87, 13-21 (1978).

Salomon, Marcinowski, Friedland, Zagorski, Biochemistry, 35:13568-13578, 1996.

Salto, Buclak, Yang, Partridge, Proc. Natl. Acad. Sci. USA, 92:10227-10231, 1995. Schellenberg, Proc. Natl. Acad. Sci. USA, 92:8552-8559, 1995.

Schagger H, von Jagow G. Tricine-Sodium Dodecyl Sulfate-Polyacrylamide Gel Electrophoresis for the separation of proteins in the range from 1 to 100 kDa. Anal. Biochem. 166, 368-379 (1987).

Schrader-Fischer G., Paganetti P. A. (1996) Brain Res. 716, 91-100. 25

Selkoe, J. Neurpathol. Exp. Neurol., 53:438-447,1994.

Selkoe, Neuron, 6:487-498, 1991.

Sigurdsson, Permanne, Soto, Wismewski, Frangione, J. Neuropath. Exp. Neuro., 59:11-17,2000.

Sipe J D. Amyloidosis. Annu. Rev. Biochem. 61:947-75 (1992).

Skovronsky, D. M., Zhang, B., Kung, M., Fung, H. F., Trojanowski, J. Q. and Lee, V. M. Proc. Natl. Acad. Sci. USA, 97, 7609-7614, 2000.

Sosnick, Jackson, Wilk, Englander, DeGrado, Proteins, 24:427-432, 1996.

Soreghan B, Kosmoski J, Glabe C. Surfactant properties of Alzheimer's A beta peptides and the mechanism ofamyloid aggregation. J. Biol. Chem. 269, 28551-4 (1994).

Soto C, Sigurdsson E M, Morelli L, Kumar R A, Castano E M, Frangione B. β-sheet breaker peptides inhibit fibrillogenesis in a rat brain model of amyloidosis: Implications for Alzheimer's therapy. Nat. Med. 4, 822-826 (1998).

Soto, Kindy, Baumann, Frangione, Biochem. Biophys. Res. Commun., 226:672-680, 1996.

Soto, Mol. Med. Today, 5:343-350,1999.

Strazielle, Ghersi-Egea, Ghiso, Dehouck, Frangione, Patlak, Fenstermacher, Gorevic, J Neuropathol. Exp. Neurol., 59:29-38, 2000.

Subramanian, R. M., Fless, G., Jones, D. M., Lynn, D. G. and Meredith, S. C., 2000.

Terzi, E., Holzemann, G. and Seelig, J. (1995). Self-association of β-amyloid peptide (1-40) in solution and binding to lipid membranes. J. Mol. Biol. 252: 633-642.

Tjenberg, L. O., Naslund, J., Lindquist, F., Johansson, J., Karlstrom, A. R., Thyberg, J., Terenius, L. and Nordstedt, C. J. Biol. Chem. 271, 8545-8548, 1996.

Tjernberg, L. O., Lilliehook, C., Callaway, D. J. E., Naslund, J., Hahne, S., Thyberg, J., Terenius, L., and Nordstedt, C. (1997) J. Biol. Chem. 272, 12601-12605.

Tomiyama, Shoji, Kataoka, Suwa, Asano, Kaneko, Endo, JBiol. Chem., 271:6839-6844, 1996.

Tonelli, A. E. (1971) On the stability of cis and trans amide bond conformations in polypeptides. J. Am. Chem. Soc. 93, 7153-7155.

Tonelli, A. E. (1974) Conformational characteristics of polypeptides containing isolated L-proline residues with cis peptide bonds. J. Mol. Biol. 86, 627-635.

Vitoux, B., Aubry, A., Cung, M. and Marraud, M. (1986). N-methyl peptides. VII. Conformational pertrrubations induced by N-methylation of model dipeptides. Int. J. Protein Peptide Res. 27, 617-632,.

Vitoux, B., Aubry, A., Cung, M., Boussard, G. and Marraud, M. Int. J. Protein Peptide 20 Res. 17, 469-479, (1981).

Walsh, D. M., Tseng, B. P., Rydel, R. E., Podlisny, M. B. and Selkoe, D. J. Biochemistry 39, 10831-10839, (2000).

Wimley, Hristova, Ladokhin, Silvestro, Axelsen, White, J M61. Biol., 277:1091-1110, 1998.

Wiberg K B, Laidig K E. Barries to Rotation Adjacent to Double Bonds. 3. The C—O Barrier in Formic Acid, Methyl Formate, Acetic Acid, and Methyl Acetate. The Origin of Ester and Amide Resonance. J. Am. Chem. Soc. 109, 5935-5943 (1987).

Wood, MacKenzie, Maleeff, Hurle, Wetzel, J Biol. Chem., 2710.84086-β4092,1996. Wood, Wetzel, Martin, Hurle, Biochemistry, 34:724-730, 1995.

Wuthrich, K. (1986). NMR of Proteins and Nucleic Acids, John Wiley and Sons, New York.

Yong W, Lomakin A, Kirkitadze M D, Teplow D B, Chen S H, Benedek G B. Structure determination of micelle-like intermediates in amyloid beta-protein fibril assembly by using small angle neutron scattering. Proc. Natl. Acad. Sci. USA. 99, 150-154 (2002).

U.S. Pat. No. 5,643,562.

U.S. Pat. No. 4,554,101.

U.S. Pat. No. 4,816,567.

U.S. Pat. No. 4,472,509.

U.S. Pat. No. 4,938,948.

What is claimed is:

1. A peptide having the following characteristics:
(a) inhibits fibrillogenesis;
(b) is a β-strand with two faces, wherein
i) a first face has hydrogen bonds; and
ii) a second face blocks or disrupts propagation of hydrogen bonding between β-strands needed to form fibrils.

2. The peptide of claim 1, wherein the second face has N-methyl amino acids in alternate positions.

3. The peptide of claim 1, wherein the second face has ester bonds at alternate positions.

4. The peptide of claim 2, wherein there are at least 2 N-methyl amino acid groups in alternate positions.

5. The peptide of claim 1, farther characterized as soluble in water.

6. The peptide of claim 1, further characterized as penetrating phospholipid bilayers.

7. Use of the peptide of claim 1 to inhibit fibrillogenesis.

8. A pharmaceutical composition comprising the peptide of claim 1, said composition inhibiting or disassembling fibrils associated with pathological states selected from the group consisting of Alzheimer's Disease, Down's Syndrome, Dutch-Type Hereditary Cerebral Hemorrhage Amyloidosis, Reactive Amyloidosis, Familial Mediterranean Fever, Familial Amyloid Nephropathy With Urticaria And Deafnless, Muckle-Wells Syndrome, Idiopathic Mycloma; Macroglobulinemia-Associated Myeloma, Familial Amyloid Polyneuropathy, Familial Amyloid Cardiomyopathy, Isolated Cardiac Amyloid, Systemic Senile Amyloidosis, Adult Onset Diabetes, Insulinoma, Isolated Atrial Amyloid, Medullary Carcinoma Of The Thyroid, Familial Amyloidosis, Hereditary Cerebral Hemorrhage With Amyloidosis, Familial Amyloidotic Polyneuropathy, Scrapie, Creutzfeldt-Jacob Disease, Gerstmann-Straussler-Scheinker Syndrome, Bovine Spongiform Encephalitis, Prion-mediated diseases, and Huntington's Disease.

9. A method for detecting fibrils in a subject, said method comprising:
(a) contacting the subject with a sample of a conjugated peptide fibril inhibitor of claim 1; and
(b) detecting the presence of fibrils by detecting the binding of the peptide to the fibrils.

10. A method for screening candidate fibrillogenesis inbhitors comprising:
(a) obtaining a sample containing fibril forming proteins;
(b) contacting the sample with a peptide composition comprising a polypeptide comprising a β-strand with a first face and a second face, whereint he first face is adapted to bind a fibril froming protein through hydrogen bonds, and the second face is adapted to block propagation of hydrogen bonds; and
(c) measuring the inhibition of fibril formation.

11. A method for preparing an inhibitor of fibrillogenesis, said method comrpsing:
(a) asdkfj
